SEQUENCE LISTING (481 sequences)

SEQ ID NO: 1 - protein, 5 aa, Artificial Sequence
  Synthetic
GSGGS

SEQ ID NO: 2 - protein, 4 aa, Artificial Sequence
  Synthetic
GGGS

SEQ ID NO: 3 - protein, 4 aa, Artificial Sequence
  Synthetic
GGSG

SEQ ID NO: 4 - protein, 5 aa, Artificial Sequence
  Synthetic
GGSGG

SEQ ID NO: 5 - protein, 5 aa, Artificial Sequence
  Synthetic
GSGSG

SEQ ID NO: 6 - protein, 5 aa, Artificial Sequence
  Synthetic
GSGGG

SEQ ID NO: 7 - protein, 5 aa, Artificial Sequence
  Synthetic
GGGSG

SEQ ID NO: 8 - protein, 5 aa, Artificial Sequence
  Synthetic
GSSSG

SEQ ID NO: 9 - protein, 13 aa, Artificial Sequence
  Synthetic
GSSGGSGGSGGSG

SEQ ID NO: 10 - protein, 11 aa, Artificial Sequence
  Synthetic
GSSGGSGGSGG

SEQ ID NO: 11 - protein, 12 aa, Artificial Sequence
  Synthetic
GSSGGSGGSGGS

SEQ ID NO: 12 - protein, 16 aa, Artificial Sequence
  Synthetic
GSSGGSGGSGGSGGGS

SEQ ID NO: 13 - protein, 10 aa, Artificial Sequence
  Synthetic
GSSGGSGGSG

SEQ ID NO: 14 - protein, 11 aa, Artificial Sequence
  Synthetic
GSSGGSGGSGS

SEQ ID NO: 15 - protein, 10 aa, Artificial Sequence
  Synthetic
GSSGGSGGGS

SEQ ID NO: 16 - protein, 5 aa, Artificial Sequence
  Synthetic
GSSGT

SEQ ID NO: 17 - protein, 4 aa, Artificial Sequence
  Synthetic
GSSG

SEQ ID NO: 18-75 - skipped (58 entries)

SEQ ID NO: 76 - protein, 18 aa, Artificial Sequence
  Synthetic
AVGLLAPPGGLSGRSDNH

SEQ ID NO: 77-87 - skipped (11 entries)

SEQ ID NO: 88 - protein, 6 aa, Artificial Sequence
  Synthetic
QGQSGQ

SEQ ID NO: 89 - protein, 8 aa, Artificial Sequence
  Synthetic
PRFKIIGG

SEQ ID NO: 90 - protein, 8 aa, Artificial Sequence
  Synthetic
PRFRIIGG

SEQ ID NO: 91 - protein, 9 aa, Artificial Sequence
  Synthetic
SSRHRRALD

SEQ ID NO: 92 - protein, 14 aa, Artificial Sequence
  Synthetic
RKSSIIIRMRDVVL

SEQ ID NO: 93 - protein, 15 aa, Artificial Sequence
  Synthetic
SSSFDKGKYKKGDDA

SEQ ID NO: 94 - protein, 15 aa, Artificial Sequence
  Synthetic
SSSFDKGKYKRGDDA

SEQ ID NO: 95 - protein, 4 aa, Artificial Sequence
  Synthetic
IEGR

SEQ ID NO: 96 - protein, 4 aa, Artificial Sequence
  Synthetic
IDGR

SEQ ID NO: 97 - protein, 7 aa, Artificial Sequence
  Synthetic
GGSIDGR

SEQ ID NO: 98 - protein, 6 aa, Artificial Sequence
  Synthetic
PLGLWA

SEQ ID NO: 99 - protein, 8 aa, Artificial Sequence
  Synthetic
GPQGIAGQ

SEQ ID NO: 100 - protein, 8 aa, Artificial Sequence
  Synthetic
GPQGLLGA

SEQ ID NO: 101 - protein, 5 aa, Artificial Sequence
  Synthetic
GIAGQ

SEQ ID NO: 102 - protein, 8 aa, Artificial Sequence
  Synthetic
GPLGIAGI

SEQ ID NO: 103 - protein, 8 aa, Artificial Sequence
  Synthetic
GPEGLRVG

SEQ ID NO: 104 - protein, 8 aa, Artificial Sequence
  Synthetic
YGAGLGVV

SEQ ID NO: 105 - protein, 8 aa, Artificial Sequence
  Synthetic
AGLGVVER

SEQ ID NO: 106 - protein, 8 aa, Artificial Sequence
  Synthetic
AGLGISST

SEQ ID NO: 107 - protein, 8 aa, Artificial Sequence
  Synthetic
EPQALAMS

SEQ ID NO: 108 - protein, 8 aa, Artificial Sequence
  Synthetic
QALAMSAI

SEQ ID NO: 109 - protein, 8 aa, Artificial Sequence
  Synthetic
AAYHLVSQ

SEQ ID NO: 110 - protein, 8 aa, Artificial Sequence
  Synthetic
MDAFLESS

SEQ ID NO: 111 - protein, 8 aa, Artificial Sequence
  Synthetic
ESLPVVAV

SEQ ID NO: 112 - protein, 8 aa, Artificial Sequence
  Synthetic
SAPAVESE

SEQ ID NO: 113 - protein, 8 aa, Artificial Sequence
  Synthetic
DVAQFVLT

SEQ ID NO: 114 - protein, 8 aa, Artificial Sequence
  Synthetic
VAQFVLTE

SEQ ID NO: 115 - protein, 8 aa, Artificial Sequence
  Synthetic
AQFVLTEG

SEQ ID NO: 116 - protein, 8 aa, Artificial Sequence
  Synthetic
PVQPIGPQ

SEQ ID NO: 117 - protein, 273 aa, Artificial Sequence
  Synthetic
GGGSGGGGSGSGGGSGGGGSGGGEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGGGTKVEIKRSGGSTITSYNVYYTKLSSSGTQVQLVQTGGGVVQPGRSLRLSCAASGSTFSSYAMSWVRQAPGKGLEWVSAISGSGGSTYYADSVKGRFTISRDNSKNTLYLQMNSLRAEDTAVYYCATNSLYWYFDLWGRGTLVTVSSAS

SEQ ID NO: 118 - protein, 264 aa, Artificial Sequence
  Synthetic
GGGSGGGGSGSGGGSGGGGSGGGQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 119-221 - skipped (103 entries)

SEQ ID NO: 222 - protein, 15 aa, Artificial Sequence
  Synthetic
LCHPAVLSAWESCSS

SEQ ID NO: 223-238 - skipped (16 entries)

SEQ ID NO: 239 - protein, 451 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 240 - protein, 219 aa, Artificial Sequence
  Synthetic
DIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 241 - DNA, 1353 bp, Artificial Sequence
  Synthetic
cagatcaccctgaaagagtccggccccaccctggtgaaacccacccagaccctgaccctgacatgcaccttctccggcttcagcctgtccacctacggcatgggcgtgggctggatcaggcagcctcctggcaaggccctggaatggctggccaacatctggtggtccgaggacaagcactactcccccagcctgaagtcccggctgaccatcaccaaggacacctccaagaaccaggtggtgctgacaatcacaaacgtggaccccgtggacaccgccacctactactgcgtgcagatcgactacggcaacgactacgccttcacctactggggccagggcacactggtgacagtgtcctccgcctccaccaagggcccctccgtgttccctctggccccttccagcaagtccacctctggcggcacagctgccctgggctgcctggtgaaagactacttccccgagcccgtgaccgtgtcctggaactctggcgccctgaccagcggagtgcacaccttccctgccgtgctgcagtcctccggcctgtactccctgtcctccgtggtgaccgtgccctccagctctctgggcacccagacctacatctgcaacgtgaaccacaagccctccaacaccaaggtggacaagaaggtggaacccaagtcctgcgacaagacccacacctgtcccccctgccctgcccctgaactgctgggcggaccttccgtgtttctgttccccccaaagcctaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtgtccaacaaggccctgcctgcccccatcgaaaagaccatctccaaggccaagggccagccccgcgagcctcaggtgtacacactgccccctagccgggaagagatgaccaagaatcaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgcagcgtgatgcacgaggccctgcacaaccactacacccagaagtccctgtccctgagccccggcaag

SEQ ID NO: 242-245 - skipped (4 entries)

SEQ ID NO: 246 - protein, 270 aa, Artificial Sequence
  Synthetic
QGQSGQGLCHPAVLSAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 247 - DNA, 810 bp, Artificial Sequence
  Synthetic
cagggacagtctggccagggcctgtgtcaccctgctgtgctgtctgcctgggagtcctgttccagcggcggaggctcctctggcggctctgctgtgggcctgctggctccacctggcggcctgtccggcagatctgacaaccacggcggctccgacatcgtgatgacccagtcccccctgtccctgcccgtgactcctggcgagcctgcctccatctcctgccggtcctccaagtccctgctgcactccaacggcatcacctacctgtactggtatctgcagaagcccggccagtcccctcagctgctgatctaccagatgtccaacctggcctccggcgtgcccgacagattctccggctctggctccggcaccgacttcaccctgaagatctcccgggtggaagccgaggacgtgggcgtgtactactgcgcccagaacctggaactgccctacaccttcggccagggcaccaagctggaaatcaagcggaccgtggccgctccctccgtgttcatcttcccaccctccgacgagcagctgaagtccggcaccgcctccgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggactgagcagccccgtgaccaagtccttcaaccggggcgagtgc

SEQ ID NO: 248-304 - skipped (57 entries)

SEQ ID NO: 305 - protein, 7 aa, Artificial Sequence
  Synthetic
QGQSGQG

SEQ ID NO: 306 - protein, 5 aa, Artificial Sequence
  Synthetic
QGQSG

SEQ ID NO: 307 - protein, 4 aa, Artificial Sequence
  Synthetic
QGQS

SEQ ID NO: 308-313 - skipped (6 entries)

SEQ ID NO: 314 - protein, 263 aa, Artificial Sequence
  Synthetic
LCHPAVLSAWESCSSGGGSSGGSAVGLLAPPGGLSGRSDNHGGSDIVMTQSPLSLPVTPGEPASISCRSSKSLLHSNGITYLYWYLQKPGQSPQLLIYQMSNLASGVPDRFSGSGSGTDFTLKISRVEAEDVGVYYCAQNLELPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 315 - DNA, 789 bp, Artificial Sequence
  Synthetic
ctgtgtcaccctgctgtgctgtctgcctgggagtcctgttccagcggcggaggctcctctggcggctctgctgtgggcctgctggctccacctggcggcctgtccggcagatctgacaaccacggcggctccgacatcgtgatgacccagtcccccctgtccctgcccgtgactcctggcgagcctgcctccatctcctgccggtcctccaagtccctgctgcactccaacggcatcacctacctgtactggtatctgcagaagcccggccagtcccctcagctgctgatctaccagatgtccaacctggcctccggcgtgcccgacagattctccggctctggctccggcaccgacttcaccctgaagatctcccgggtggaagccgaggacgtgggcgtgtactactgcgcccagaacctggaactgccctacaccttcggccagggcaccaagctggaaatcaagcggaccgtggccgctccctccgtgttcatcttcccaccctccgacgagcagctgaagtccggcaccgcctccgtggtctgcctgctgaacaacttctacccccgcgaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaagtgacccaccagggactgagcagccccgtgaccaagtccttcaaccggggcgagtgc

SEQ ID NO: 316-318 - skipped (3 entries)

SEQ ID NO: 319 - DNA, 21 bp, Artificial Sequence
  Synthetic
cagggacagtctggccagggc

SEQ ID NO: 320-358 - skipped (39 entries)

SEQ ID NO: 359 - protein, 6 aa, Artificial Sequence
  Synthetic
GQSGQG

SEQ ID NO: 360 - protein, 5 aa, Artificial Sequence
  Synthetic
QSGQG

SEQ ID NO: 361 - protein, 4 aa, Artificial Sequence
  Synthetic
SGQG

SEQ ID NO: 362-478 - skipped (117 entries)

SEQ ID NO: 479 - protein, 8 aa, Artificial Sequence
  Synthetic
GGGSSGGS

SEQ ID NO: 480 - protein, 450 aa, Artificial Sequence
  Synthetic
QITLKESGPTLVKPTQTLTLTCTFSGFSLSTYGMGVGWIRQPPGKALEWLANIWWSEDKHYSPSLKSRLTITKDTSKNQVVLTITNVDPVDTATYYCVQIDYGNDYAFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 481 - DNA, 1350 bp, Artificial Sequence
  Synthetic
cagatcaccctgaaagagtccggccccaccctggtgaaacccacccagaccctgaccctgacatgcaccttctccggcttcagcctgtccacctacggcatgggcgtgggctggatcaggcagcctcctggcaaggccctggaatggctggccaacatctggtggtccgaggacaagcactactcccccagcctgaagtcccggctgaccatcaccaaggacacctccaagaaccaggtggtgctgacaatcacaaacgtggaccccgtggacaccgccacctactactgcgtgcagatcgactacggcaacgactacgccttcacctactggggccagggcacactggtgacagtgtcctccgcctccaccaagggcccctccgtgttccctctggccccttccagcaagtccacctctggcggcacagctgccctgggctgcctggtgaaagactacttccccgagcccgtgaccgtgtcctggaactctggcgccctgaccagcggagtgcacaccttccctgccgtgctgcagtcctccggcctgtactccctgtcctccgtggtgaccgtgccctccagctctctgggcacccagacctacatctgcaacgtgaaccacaagccctccaacaccaaggtggacaagaaggtggaacccaagtcctgcgacaagacccacacctgtcccccctgccctgcccctgaactgctgggcggaccttccgtgtttctgttccccccaaagcctaaggacaccctgatgatctcccggacccccgaagtgacctgcgtggtggtggacgtgtcccacgaggaccctgaagtgaagttcaattggtacgtggacggcgtggaagtgcacaacgccaagaccaagcccagagaggaacagtacaactccacctaccgggtggtgtctgtgctgaccgtgctgcaccaggactggctgaacggcaaagagtacaagtgcaaggtgtccaacaaggccctgcctgcccccatcgaaaagaccatctccaaggccaagggccagccccgcgagcctcaggtgtacacactgccccctagccgggaagagatgaccaagaatcaggtgtccctgacctgtctggtgaaaggcttctacccctccgatatcgccgtggaatgggagtccaacggccagcccgagaacaactacaagaccaccccccctgtgctggactccgacggctcattcttcctgtactccaagctgaccgtggacaagtcccggtggcagcagggcaacgtgttctcctgcagcgtgatgcacgaggccctgcacaaccactacacccagaagtccctgtccctgagccccggc

(19) United States
(12) Patent Application Publication
(10) Pub. No.: US 2022/0023439 A1
(43) Pub. Date: Jan. 27, 2022

(54) ACTIVATABLE ANTI-CD166 ANTIBODIES AND METHODS OF USE THEREOF

(71) Applicant: CytomX Therapeutics, Inc., South San Francisco, CA (US)

(72) Inventors: Lori CARMAN, South San Francisco, CA (US); Rachel HUMPHREY, South San Francisco, CA (US); W. Michael KAVANAUGH, South San Francisco, CA (US); Jonathan A. TERRETT, South San Francisco, CA (US); Annie Yang WEAVER, South San Francisco, CA (US); Matthias WILL, South San Francisco, CA (US)

(21) Appl. No.: 17/290,694

(22) PCT Filed: Nov. 1, 2019
(86) PCT No.: PCT/US2019/059363

§ 371 Date: Apr. 30, 2021

Related U.S. Application Data
(60) Provisional application No. 62/826,465, filed on Mar. 29, 2019.
(60) Provisional application No. 62/810,173, filed on Feb. 25, 2019.
(60) Provisional application No. 62/789,403, filed on Jan. 7, 2019.
(60) Provisional application No. 62/788,714, filed on Jan. 4, 2019.
(60) Provisional application No. 62/755,247, filed on Nov. 2, 2018.

(51) Int. Cl. A61K 47/68; A61K 47/64; A61K 47/65

(52) U.S. Cl. CPC A61K 47/6851; A61K 47/65; A61K 47/64; A61K 47/6809

(57) ABSTRACT

Provided herein are activatable antibodies that when activated specifically bind to CD166 and conjugated activatable antibodies that specifically bind to CD166. Also provided are methods of making and using these activatable antibodies in a variety of therapeutic, diagnostic and prophylactic indications.

### FIELD OF THE INVENTION

This invention generally relates to specific dosing regimens for administering anti-CD166 conjugated activatable antibodies for the treatment of cancer.

### REFERENCE TO SEQUENCE LISTING

The “Sequence Listing” submitted electronically concurrently herewith pursuant to 37 C.F.R. § 1.821 in computer readable form (CFR) via EFS-Web as file name “CYTX-059-PCT_ST25” is incorporated herein by reference. The electronic copy of the Sequence Listing was created on Oct. 29, 2019, and the disk size is 49 kilobytes.

### BACKGROUND OF THE INVENTION

Antibody-based therapies have proven to be effective treatments for several diseases, including cancers, but in some cases, toxicities due to broad target expression have limited their therapeutic effectiveness. In addition, antibody-based therapeutics have exhibited other limitations such as rapid clearance from the circulation following administration.

In the realm of small molecule therapeutics, strategies have been developed to provide prodrugs of an active chemical entity. Such prodrugs are administered in a relatively inactive (or significantly less active) form. Once administered, the prodrug is metabolized in vivo into the active compound. Such prodrug strategies can provide for increased selectivity of the drug for its intended target and for a reduction of adverse effects.

Accordingly, there is a continued need in the field of antibody-based therapeutics for antibodies that mimic the desirable characteristics of the small molecule prodrug.

### SUMMARY OF THE INVENTION

In one aspect of the invention, provided herein is a method of treating, alleviating a symptom of, or delaying the progression of a cancer in a subject, the method comprising administering a therapeutically effective amount of an activatable antibody (AA) conjugated to an agent to a subject in need thereof, wherein the subject is administered the AA conjugated to an agent at a dose of greater than 6 mg/kg to about 10 mg/kg, wherein the AA comprises (a) an antibody or an antigen binding fragment thereof (AB) that specifically binds to mammalian CD166, wherein the AB comprises a heavy chain comprising an amino acid sequence of SEQ ID NO: 480, and a light chain comprising an amino acid sequence of SEQ ID NO: 240; (b) a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and (c) a cleavable moiety (CM) coupled to the AB, wherein the CM is a polypeptide that functions as a substrate for a protease, and wherein the CM comprises the amino acid sequence of SEQ ID NO: 76. In some embodiments, the light chain comprises the sequence of SEQ ID NO: 314; in some embodiments, the light chain comprises the sequence of SEQ ID NO: 246. In some embodiments, the cancer is breast carcinoma, castration-resistant prostate carcinoma, cholangiocarcinoma, endometrial carcinoma, epithelial ovarian carcinoma, head and neck squamous cell carcinoma, or non-small cell lung cancer.

In a related aspect of the invention, provided herein is a method of inhibiting or reducing the growth, proliferation, or metastasis of cells expressing CD166 in a subject, comprising administering a therapeutically effective amount of an activatable antibody (AA) conjugated to an agent to a subject in need thereof, wherein the subject is administered the AA conjugated to an agent at a dose of greater than 6 mg/kg to about 10 mg/kg, wherein the AA comprises (a) an antibody or an antigen binding fragment thereof (AB) that specifically binds to mammalian CD166, wherein the AB comprises a heavy chain comprising an amino acid sequence of SEQ ID NO: 480, and a light chain comprising an amino acid sequence of SEQ ID NO: 240; (b) a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and (c) a cleavable moiety (CM) coupled to the AB, wherein the CM is a polypeptide that functions as a substrate for a protease, and wherein the CM comprises the amino acid sequence of SEQ ID NO: 76. In some embodiments, the light chain comprises the sequence of SEQ ID NO: 314; in some embodiments, the light chain comprises the sequence of SEQ ID NO: 246.

In a further related aspect of the invention, provided herein is an activatable antibody (AA) conjugated to an agent for use in treating, alleviating a symptom of, or delaying the progression of a cancer in a subject, wherein the AA comprises (a) an antibody or an antigen binding fragment thereof (AB) that specifically binds to mammalian CD166, wherein the AB comprises a heavy chain comprising an amino acid sequence of SEQ ID NO: 480, and a light chain comprising an amino acid sequence of SEQ ID NO: 240; (b) a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and (c) a cleavable moiety (CM) coupled to the AB, wherein the CM is a polypeptide that functions as a substrate for a protease, and wherein the CM comprises the amino acid sequence of SEQ ID NO: 76. In some embodiments, the light chain comprises the sequence of SEQ ID NO: 314; in some embodiments, the light chain comprises the sequence of SEQ ID NO: 246. In some embodiments, the cancer is breast carcinoma, castration-resistant prostate carcinoma, cholangiocarcinoma, endometrial carcinoma, epithelial ovarian carcinoma, head and neck squamous cell carcinoma, or non-small cell lung cancer. In some embodiments, the cancer is breast carcinoma, prostate carcinoma, cholangiocarcinoma, endometrial carcinoma, ovarian carcinoma, head and neck carcinoma, or lung cancer. The AA is for administration to the subject in a therapeutically effective amount. In some embodiments, the therapeutically effective amount is a dose of greater than 6 mg/kg to about 10 mg/kg.

In a still further related aspect of the invention, provided herein is an activatable antibody (AA) conjugated to an agent for use in inhibiting or reducing the growth, proliferation, or metastasis of cells expressing CD166 for the treatment of cancer in a subject, wherein the AA comprises (a) an antibody or an antigen binding fragment thereof (AB) that specifically binds to mammalian CD166, wherein the AB comprises a heavy chain comprising an amino acid sequence of SEQ ID NO: 480, and a light chain comprising an amino acid sequence of SEQ ID NO: 240; (b) a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and (c) a cleavable moiety (CM) coupled to the AB, wherein the CM is a polypeptide that functions as a substrate for a protease, and wherein the CM comprises the amino acid sequence of SEQ ID NO: 76. In some embodiments, the light chain comprises the sequence of SEQ ID NO: 314; in some embodiments, the light chain comprises the sequence of SEQ ID NO: 246. The AA is for administration in a therapeutically effective amount to a subject in need thereof. In some embodiments, the therapeutically effective amount is a dose of greater than 6 mg/kg to about 10 mg/kg.

In some embodiments, the subject suffers from breast carcinoma, castration-resistant prostate carcinoma, cholangiocarcinoma, endometrial carcinoma, epithelial ovarian carcinoma, head and neck squamous cell carcinoma, or non-small cell lung cancer. In some embodiments, the cells are breast cells, prostate cells, endometrial cells, ovarian cells, head or neck cells, bile duct cells, or lung cells.

In some embodiments, the agent conjugated to the AA is a maytansinoid or derivative thereof; for example, the agent conjugated to the AA is DM4; in some embodiments, the DM4 is conjugated to the AA via a linker; in some embodiments, the linker comprises an SPBD (N-succinimidyl-4-(2-pyridyldithio) butanoate) moiety.

In some embodiments, the AB is linked to the CM, for example via a linking peptide. In some embodiments, the MM is linked to the CM such that the AA in an uncleaved state comprises the structural arrangement from N-terminus to C-terminus as follows: MM-CM-AB or AB-CM-MM. In some embodiments, the AA comprises a linking peptide between the MM and the CM; for example, the linking peptide can comprise the amino acid sequence of SEQ ID NO: 479. In some embodiments, the AA comprises a linking peptide between the CM and the AB; for example, the linking peptide comprises the amino acid sequence of SEQ ID NO: 15. In some embodiments, the AA comprises a linking peptide between the CM and the AB; for example, the linking peptide comprises the amino acid sequence of GGS.

In some embodiments, the AA comprises a first linking peptide (LP1) and a second linking peptide (LP2), and wherein the AA in the uncleaved state has the structural arrangement from N-terminus to C-terminus as follows: MM-LP1-CM-LP2-AB or AB-LP2-CM-LP1-MM.

In some embodiments, the light chain is linked to a spacer at its N-terminus; in some embodiments, the spacer comprises the amino acid sequence of SEQ ID NO: 305; In some embodiments, the MM and CM are linked to the light chain; in some embodiments, the MM is linked to the CM such that the AA in an uncleaved state comprises the structural arrangement from N-terminus to C-terminus on its light chain as follows: spacer-MM-LP1-CM-LP2-light chain; in some embodiments, the spacer comprises the amino acid sequence of SEQ ID NO: 305, LP1 comprises the amino acid sequence of SEQ ID NO: 479, and LP2 comprises the amino acid sequence of SEQ ID NO: 15. In some embodiments, the light chain is linked to a spacer at its N-terminus; in some embodiments, the spacer comprises the amino acid sequence of SEQ ID NO: 305; In some embodiments, the MM and CM are linked to the light chain; in some embodiments, the MM is linked to the CM such that the AA in an uncleaved state comprises the structural arrangement from N-terminus to C-terminus on its light chain as follows: spacer-MM-LP1-CM-LP2-light chain; in some embodiments, the spacer comprises the amino acid sequence of SEQ ID NO: 305, LP1 comprises the amino acid sequence of SEQ ID NO: 479, and LP2 comprises the amino acid sequence of GGS.

In some embodiments, the subject is at least 18 years of age; in some embodiments, the subject has an ECOG performance status of 0-1; in some embodiments, the subject has a histologically confirmed diagnosis of an active metastatic cancer; in some embodiments, the subject has a histologically confirmed diagnosis of a locally advanced unresectable solid tumor; in some embodiments, the subject has a life expectancy of greater than 3 months at the time of administration.

In some embodiments, the subject has a breast carcinoma; in some embodiments, the breast carcinoma is ER+; in some embodiments, the subject has received prior anti-hormonal therapy and has experienced disease progression; in another embodiment the subject has a triple negative breast cancer and has underwent at least two prior lines of therapy; in another embodiment the subject has not had a history of acute or chronic corneal disease.

In some embodiments, the subject has castration-resistant prostate carcinoma, in some embodiments, the subject has received at least one prior therapy.

In some embodiments, the subject has cholangiocarcinoma. In some embodiments, the subject has failed at least one prior line of gemcitabine-containing regimen.

In some embodiments, the subject has endometrial carcinoma; in some embodiments, the subject has received at least one platinum-containing regimen for extra-uterine or advanced disease.

In some embodiments, the subject has epithelial ovarian carcinoma. In some embodiments, the subject has a platinum-resistant carcinoma; in some embodiments, the subject has a platinum refractory ovarian carcinoma; in some embodiments, the subject has a BRCA mutation and is refractory to PARP inhibitors. In other embodiments the subject has a non-BRCA mutation.

In some embodiments, the subject has head and neck small cell carcinoma (HNSCC); in some embodiments, the subject has received more than one platinum-containing regimen; in some embodiments, the subject has received more than one PD-1/PD-L1 inhibitor.

In some embodiments, the subject has non-small cell lung cancer (NSCLC), in some embodiments, the subject has received at least one platinum-containing regimen; in some embodiments, the subject has received at least one PD-1/PD-L1 inhibitor. In some embodiments, the subject has received at least one checkpoint inhibitor.

In some embodiments, the subject has a skin lesion. In some embodiments, the skin lesion is a skin metastasis.

In some embodiments, the subject is administered the AA which is conjugated to an agent at a dose of greater than 6 mg/kg to about 10 mg/kg; for example, the administered dose is greater than 6 mg/kg; the administered dose is about 7 mg/kg; the administered dose is about 8 mg/kg; the administered dose is about 9 mg/kg; the administered dose is about 10 mg/kg.

In some embodiments, the subject is administered the AA which is conjugated to an agent at a dose of greater than 6 mg/kg to about 7 mg/kg; for example, the administered dose is about 7 mg/kg to about 8 mg/kg; the administered dose is about 8 mg/kg to about 9 mg/kg; the administered dose is about 9 mg/kg to about 10 mg/kg; the administered dose is greater than 6 mg/kg to about 8 mg/kg; the administered dose is about 7 mg/kg to about 9 mg/kg; the administered dose is about 8 mg/kg to about 10 mg/kg.

In some embodiments, the subject is administered the AA conjugated to an agent at a fixed dose of greater than 240 mg to about 1000 mg or at a fixed dose of greater than 240 mg to about 400 mg or at a fixed dose of greater than 600 mg to about 1000 mg or at a fixed dose of greater than 240 mg to greater than 600 mg; for example, the administered fixed dose is about 280 mg to about 700 mg; the administered fixed dose is about 320 mg to about 800 mg; the administered fixed dose is about 360 mg to about 900 mg; the administered fixed dose is about 400 mg to about 1000 mg; the administered fixed dose is greater than 240 mg to about 280 mg; the administered fixed dose is about 280 mg to about 320 mg; the administered fixed dose is about 320 mg to about 360 mg; the administered fixed dose is about 360 mg to about 400 mg; the administered fixed dose is greater than 600 mg to about 700 mg; the administered fixed dose is about 700 mg to about 800 mg; the administered fixed dose is about 800 mg to about 900 mg; the administered fixed dose is about 900 mg to about 1000 mg; the administered fixed dose is greater than 240 mg to about 320 mg; the administered fixed dose is about 280 mg to about 360 mg; the administered fixed dose is about 320 mg to about 400 mg; the administered fixed dose is greater than 600 mg to about 800 mg; the administered fixed dose is about 700 mg to about 900 mg; the administered fixed dose is about 800 mg to about 1000 mg.

In some embodiments, the subject is administered the AA conjugated to an agent at a fixed dose of greater than 360 mg to about 600 mg; for example, the administered fixed dose is greater than 360 mg to about 420 mg; the administered fixed dose is about 420 mg to about 480 mg; the administered fixed dose is about 480 mg to about 540 mg; the administered fixed dose is about 540 mg to about 600 mg; the administered fixed dose is greater than 360 mg to about 480 mg; the administered fixed dose is about 420 mg to about 540 mg; the administered fixed dose is about 480 mg to about 600 mg.

In some embodiments, the subject is administered the AA conjugated to an agent at a fixed dose of greater than 480 mg to about 800 mg; for example, the administered fixed dose is greater than 480 mg to about 560 mg; the administered fixed dose is about 560 mg to about 640 mg; the administered fixed dose is about 640 mg to about 720 mg; the administered fixed dose is about 720 mg to about 800 mg; the administered fixed dose is greater than 480 mg to about 560 mg; the administered fixed dose is about 560 mg to about 720 mg; the administered fixed dose is about 640 mg to about 800 mg.

In some embodiments, the subject is administered the AA conjugated to an agent intravenously; in some embodiments, the subject is administered the AA conjugated to an agent intravenously every 21 days; in some embodiments, the subject is administered the AA conjugated to an agent intravenously every 14 days.

In some embodiments, the subject is administered the AA conjugated to an agent with a dosage based on the subject's actual body weight. In some embodiments, the subject is administered the AA conjugated to an agent with a dosage based on the subject's adjusted ideal body weight.

In some embodiments, the subject is administered with one or more prophylactic treatments to reduce or prevent ocular adverse events; in some embodiments, the one or more prophylactic treatments are administered daily; in some embodiments, the prophylactic treatment is one or more treatments selected from the group consisting of: lubricating artificial tears, brimonidine tartrate ophthalmic solution, application of a cool compress for the eyes, and topical steroid drops.

### BRIEF DESCRIPTION OF THE DRAWINGS

FIG. 1 depicts activatable anti-CD166 antibody drug conjugate being preferentially activated in the tumor microenvironment, where tumor-specific proteases are present.

FIG. 2 demonstrates expression of CD166 in human tumor samples by immunohistochemistry (IHC).

FIG. 3 shows the anti-tumor activity of an activatable anti-CD166 antibody drug conjugate and an anti-CD166 antibody drug conjugate in a mouse tumor model of TNBC. Also shown is CD166 expression by immunohistochemistry (IHC). (AADC=activatable anti-CD166 antibody drug conjugate; ADC=anti-CD166 drug conjugate)

FIG. 4 shows the anti-tumor activity of an activatable anti-CD166 antibody drug conjugate and an anti-CD166 antibody drug conjugate in a mouse tumor model of non-small cell lung cancer. Also shown is CD166 expression by IHC.

FIG. 5 shows the anti-tumor activity of an activatable anti-CD166 antibody drug conjugate and an anti-CD166 antibody drug conjugate in a mouse patient-derived xenograft (PDX) model for ovarian cancer. Also shown is CD166 expression by IHC.

FIG. 6 illustrates the Part A and Part B clinical trial design for an activatable anti-CD166 antibody drug conjugate.

FIGS. 7A-7B demonstrates preferential activation of an activatable anti-CD166 antibody in tumors.

FIGS. 8A-8B demonstrates separation of intact and activated forms of an activatable anti-CD166 antibody drug conjugate partially activated by matriptase (MT-SP1) or MMP14.

FIGS. 9A-9F shows exemplary pharmacokinetic data of serum levels of various analytes over time following administration of an activatable anti-CD166 antibody drug conjugate in human subjects.

FIGS. 10A-10D shows exemplary results of best changes in tumor lesion measurements from baseline in human subjects following administration of activatable anti-CD166 antibody drug conjugate of the present disclosure.

FIGS. 11A-11C shows exemplary results of best changes in tumor lesion measurements from baseline in human subjects with breast cancer following administration of activatable anti-CD166 antibody drug conjugate of the present disclosure.

FIGS. 12A and 12B shows exemplary results of best changes in tumor lesion measurements from baseline in human subjects following administration of activatable anti-CD166 antibody drug conjugate of the present disclosure as categorized on their level of CD166 expression in the patients' tumors.

### DETAILED DESCRIPTION OF THE INVENTION

The present invention provides activatable monoclonal antibodies that specifically bind CD166, also known as activated leukocyte cell adhesion molecule (ALCAM). In some embodiments, the activatable monoclonal antibodies are internalized by CD166-containing cells. CD166 is a cell adhesion molecule that binds CD6, a cell surface receptor that belongs to the scavenger receptor cysteine-rich (SRCR) protein superfamily (SRCRSF). CD166 is known to be associated with cell-cell and cell-matrix interactions, cell adhesion, cell migration, and T-cell activation and proliferation. Aberrant expression and/or activity of CD166 and CD166-related signaling has been implicated in the pathogenesis of many diseases and disorders, such as cancer, inflammation, and autoimmunity. For example, CD166 is highly expressed in a variety of cancer types such as, for example, prostate cancer, breast cancer, lung cancer such as NSCLC and/or SCLC, oropharyngeal cancer, cervical cancer, and head and neck cancer such as HNSCC.

The disclosure provides activatable anti-CD166 antibodies that are useful in methods of treating, preventing, delaying the progression of, ameliorating and/or alleviating a symptom of a disease or disorder associated with aberrant CD166 expression and/or activity. For example, the activatable anti-CD166 antibodies are used in methods of treating, preventing, delaying the progression of, ameliorating and/or alleviating a symptom of a cancer or other neoplastic condition.

The disclosure provides activatable anti-CD166 antibodies that are useful in methods of treating, preventing, delaying the progression of, ameliorating and/or alleviating a symptom of a disease or disorder associated with cells expressing CD166. In some embodiments, the cells are associated with aberrant CD166 expression and/or activity. In some embodiments, the cells are associated with normal CD166 expression and/or activity. For example, the activatable anti-CD166 antibodies are used in methods of treating, preventing, delaying the progression of, ameliorating and/or alleviating a symptom of a cancer or other neoplastic condition.

The disclosure provides activatable anti-CD166 antibodies that are useful in methods of treating, preventing, delaying the progression of, ameliorating and/or alleviating a symptom of a disease or disorder in which diseased cells express CD166. In some embodiments, the diseased cells are associated with aberrant CD166 expression and/or activity. In some embodiments, the diseased cells are associated with normal CD166 expression and/or activity. For example, the activatable anti-CD166 antibodies are used in methods of treating, preventing, delaying the progression of, ameliorating and/or alleviating a symptom of a cancer or other neoplastic condition.

The activatable anti-CD166 antibodies include an antibody or antigen-binding fragment thereof that specifically binds CD166 coupled to a masking moiety (MM), such that coupling of the MM reduces the ability of the antibody or antigen-binding fragment thereof to bind CD166. The MM is coupled to the antibody/antigen-binding fragment via a sequence that includes a substrate for a protease (cleavable moiety, CM), for example, a protease that is co-localized with CD166 at a treatment site in a subject.

### Definitions

Unless otherwise defined, scientific and technical terms used in connection with the present disclosure shall have the meanings that are commonly understood by those of ordinary skill in the art. The term “a” entity or “an” entity refers to one or more of that entity. For example, a compound refers to one or more compounds. As such, the terms “a”, “an”, “one or more” and “at least one” can be used interchangeably. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

As utilized in accordance with the present disclosure, the following terms, unless otherwise indicated, shall be understood to have the following meanings:

As used herein, the term “antibody” refers to immunoglobulin molecules and immunologically active, e.g., antigen-binding, portions of immunoglobulin (Ig) molecules, i.e., molecules that contain an antigen binding site that specifically binds (immunoreacts with) an antigen. By “specifically bind” or “immunoreacts with” or “immunospecifically bind” is meant that the antibody reacts with one or more antigenic determinants of the desired antigen and does not react with other polypeptides or binds at much lower affinity (K_{d}>10⁻⁶). Antibodies include, but are not limited to, polyclonal, monoclonal, chimeric, domain antibody, single chain, Fab, and F(ab′)₂ fragments, scFvs, and a Fab expression library.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “monoclonal antibody” (mAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

The term “antigen-binding site” or “binding portion” refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences that are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989).

As used herein, the term “epitope” includes any protein determinant capable of specific binding to an immunoglobulin, an scFv, or a T-cell receptor. The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. For example, antibodies may be raised against N-terminal or C-terminal peptides of a polypeptide. An antibody is said to specifically bind an antigen when the dissociation constant is ≤1 μM; in some embodiments, ≤100 nM and in some embodiments, ≤10 nM.

As used herein, the terms “specific binding,” “immunological binding,” and “immunological binding properties” refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361:186-87 (1993)). The ratio of K_{off}/Kₒₙ enables the cancellation of all parameters not related to affinity and is equal to the dissociation constant K_{d}. (See, generally, Davies et al. (1990) Annual Rev Biochem 59:439-473). An antibody of the present disclosure is said to specifically bind to the target, when the binding constant (K_{d}) is ≤1 μM, in some embodiments ≤100 nM, in some embodiments ≤10 nM, and in some embodiments ≤100 pM to about 1 pM, as measured by assays such as radioligand binding assays or similar assays known to those skilled in the art.

The term “isolated polynucleotide” as used herein shall mean a polynucleotide of genomic, cDNA, or synthetic origin or some combination thereof, which by virtue of its origin the “isolated polynucleotide” (1) is not associated with all or a portion of a polynucleotide in which the “isolated polynucleotide” is found in nature, (2) is operably linked to a polynucleotide which it is not linked to in nature, or (3) does not occur in nature as part of a larger sequence. Polynucleotides in accordance with the disclosure include the nucleic acid molecules encoding the heavy chain immunoglobulin molecules shown herein, and nucleic acid molecules encoding the light chain immunoglobulin molecules shown herein.

The term “isolated protein” referred to herein means a protein of cDNA, recombinant RNA, or synthetic origin or some combination thereof, which by virtue of its origin, or source of derivation, the “isolated protein” (1) is not associated with proteins found in nature, (2) is free of other proteins from the same source, e.g., free of murine proteins, (3) is expressed by a cell from a different species, or (4) does not occur in nature.

The term “polypeptide” is used herein as a generic term to refer to native protein, fragments, or analogs of a polypeptide sequence. Hence, native protein fragments, and analogs are species of the polypeptide genus. Polypeptides in accordance with the disclosure comprise the heavy chain immunoglobulin molecules shown herein, and the light chain immunoglobulin molecules shown herein, as well as antibody molecules formed by combinations comprising the heavy chain immunoglobulin molecules with light chain immunoglobulin molecules, such as kappa light chain immunoglobulin molecules, and vice versa, as well as fragments and analogs thereof.

The term “naturally-occurring” as used herein as applied to an object refers to the fact that an object can be found in nature. For example, a polypeptide or polynucleotide sequence that is present in an organism (including viruses) that can be isolated from a source in nature and that has not been intentionally modified by man in the laboratory or otherwise is naturally-occurring.

The term “operably linked” as used herein refers to positions of components so described are in a relationship permitting them to function in their intended manner. A control sequence “operably linked” to a coding sequence is ligated in such a way that expression of the coding sequence is achieved under conditions compatible with the control sequences.

The term “control sequence” as used herein refers to polynucleotide sequences that are necessary to affect the expression and processing of coding sequences to which they are ligated. The nature of such control sequences differs depending upon the host organism in prokaryotes, such control sequences generally include promoter, ribosomal binding site, and transcription termination sequence in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences. The term “polynucleotide” as referred to herein means nucleotides of at least 10 bases in length, either ribonucleotides or deoxynucleotides or a modified form of either type of nucleotide. The term includes single and double stranded forms of DNA.

The term oligonucleotide referred to herein includes naturally occurring, and modified nucleotides linked together by naturally occurring, and non-naturally occurring oligonucleotide linkages. Oligonucleotides are a polynucleotide subset generally comprising a length of 200 bases or fewer. In some embodiments, oligonucleotides are 10 to 60 bases in length and in some embodiments, 12, 13, 14, 15, 16, 17, 18, 19, or 20 to 40 bases in length. Oligonucleotides are usually single stranded, e.g., for probes, although oligonucleotides may be double stranded, e.g., for use in the construction of a gene mutant. Oligonucleotides of the disclosure are either sense or antisense oligonucleotides.

The term “naturally occurring nucleotides” referred to herein includes deoxyribonucleotides and ribonucleotides. The term “modified nucleotides” referred to herein includes nucleotides with modified or substituted sugar groups and the like. The term “oligonucleotide linkages” referred to herein includes oligonucleotide linkages such as phosphorothioate, phosphorodithioate, phosphoroselerloate, phosphorodiselenoate, phosphoroanilothioate, phoshoraniladate, phosphoronmidate, and the like. See e.g., LaPlanche et al. Nucl. Acids Res. 14:9081 (1986); Stec et al. J. Am. Chem. Soc. 106:6077 (1984), Stein et al. Nucl. Acids Res. 16:3209 (1988), Zon et al. Anti Cancer Drug Design 6:539 (1991); Zon et al. Oligonucleotides and Analogues: A Practical Approach, pp. 87-108 (F. Eckstein, Ed., Oxford University Press, Oxford England (1991)); Stec et al. U.S. Pat. No. 5,151,510; Uhlmann and Peyman Chemical Reviews 90:543 (1990). An oligonucleotide can include a label for detection, if desired.

As used herein, the twenty conventional amino acids and their abbreviations follow conventional usage. See Immunology—A Synthesis (2nd Edition, E. S. Golub and D. R. Green, Eds., Sinauer Associates, Sunderland, Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present disclosure. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the left-hand direction is the amino terminal direction and the right-hand direction is the carboxy-terminal direction, in accordance with standard usage and convention.

Similarly, unless specified otherwise, the left-hand end of single-stranded polynucleotide sequences is the 5′ end the left-hand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and that are 5′ to the 5′ end of the RNA transcript are referred to as “upstream sequences”, sequence regions on the DNA strand having the same sequence as the RNA and that are 3′ to the 3′ end of the RNA transcript are referred to as “downstream sequences”.

As applied to polypeptides, the term “substantial identity” means that two peptide sequences, when optimally aligned, such as by the programs GAP or BESTFIT using default gap weights, share at least 80 percent sequence identity, in some embodiments, at least 90 percent sequence identity, in some embodiments, at least 95 percent sequence identity, and in some embodiments, at least 99 percent sequence identity.

In some embodiments, residue positions that are not identical differ by conservative amino acid substitutions.

As discussed herein, minor variations in the amino acid sequences of antibodies or immunoglobulin molecules are contemplated as being encompassed by the present disclosure, providing that the variations in the amino acid sequence maintain at least 75%, in some embodiments, at least 80%, 90%, 95%, and in some embodiments, 99%. In particular, conservative amino acid replacements are contemplated. Conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Suitable amino- and carboxy-termini of fragments or analogs occur near boundaries of functional domains. Structural and functional domains can be identified by comparison of the nucleotide and/or amino acid sequence data to public or proprietary sequence databases. In some embodiments, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the disclosure.

Suitable amino acid substitutions are those that: (1) reduce susceptibility to proteolysis, (2) reduce susceptibility to oxidation, (3) alter binding affinity for forming protein complexes, (4) alter binding affinities, and (5) confer or modify other physicochemical or functional properties of such analogs. Analogs can include various muteins of a sequence other than the naturally-occurring peptide sequence. For example, single or multiple amino acid substitutions (for example, conservative amino acid substitutions) may be made in the naturally-occurring sequence (for example, in the portion of the polypeptide outside the domain(s) forming intermolecular contacts. A conservative amino acid substitution should not substantially change the structural characteristics of the parent sequence (e.g., a replacement amino acid should not tend to break a helix that occurs in the parent sequence or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

The term “polypeptide fragment” as used herein refers to a polypeptide that has an amino terminal and/or carboxy-terminal deletion and/or one or more internal deletion(s), but where the remaining amino acid sequence is identical to the corresponding positions in the naturally-occurring sequence deduced, for example, from a full length cDNA sequence. Fragments typically are at least 5, 6, 8 or 10 amino acids long, in some embodiments, at least 14 amino acids long, in some embodiments, at least 20 amino acids long, usually at least 50 amino acids long, and in some embodiments, at least 70 amino acids long. The term “analog” as used herein refers to polypeptides that are comprised of a segment of at least 25 amino acids that has substantial identity to a portion of a deduced amino acid sequence and that has specific binding to the target, under suitable binding conditions. Typically, polypeptide analogs comprise a conservative amino acid substitution (or addition or deletion) with respect to the naturally-occurring sequence. Analogs typically are at least 20 amino acids long, in some embodiments, at least 50 amino acids long or longer, and can often be as long as a full-length naturally occurring polypeptide.

The term “agent” is used herein to denote a chemical compound, a mixture of chemical compounds, a biological macromolecule, or an extract made from biological materials.

As used herein, the terms “label” or “labeled” refers to incorporation of a detectable marker, e.g., by incorporation of a radiolabeled amino acid or attachment to a polypeptide of biotinyl moieties that can be detected by marked avidin (e.g., streptavidin containing a fluorescent marker or enzymatic activity that can be detected by optical or calorimetric methods). In certain situations, the label or marker can also be therapeutic. Various methods of labeling polypeptides and glycoproteins are known in the art and may be used. Examples of labels for polypeptides include, but are not limited to, the following: radioisotopes or radionuclides (e.g., ³H, ¹⁴C, ¹⁵N, ³⁵S, ⁹⁰Y, ⁹⁹Tc, ¹¹¹In, ¹²⁵I, ¹³¹I), fluorescent labels (e.g., FITC, rhodamine, lanthanide phosphors), enzymatic labels (e.g., horseradish peroxidase, p-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance. The term “pharmaceutical agent or drug” as used herein refers to a chemical compound or composition capable of inducing a desired therapeutic effect when properly administered to a subject.

Other chemistry terms herein are used according to conventional usage in the art, as exemplified by The McGraw-Hill Dictionary of Chemical Terms (Parker, S., Ed., McGraw-Hill, San Francisco (1985)).

As used herein, “substantially pure” means an object species is the predominant species present (i.e., on a molar basis it is more abundant than any other individual species in the composition), and in some embodiments, a substantially purified fraction is a composition wherein the object species comprises at least about 50 percent (on a molar basis) of all macromolecular species present.

Generally, a substantially pure composition will comprise more than about 80 percent of all macromolecular species present in the composition, in some embodiments, more than about 85%, 90%, 95%, and 99%. In some embodiments, the object species is purified to essential homogeneity (contaminant species cannot be detected in the composition by conventional detection methods) wherein the composition consists essentially of a single macromolecular species.

The term subject human and veterinary subjects.

### Activatable Antibodies (AAs)

The disclosure provides AAs that include an antibody or antigen-binding fragment thereof that specifically binds mammalian CD166 (AB).

In some embodiments, the mammalian CD166 is selected from the group consisting of a human CD166 and a cynomolgus monkey CD166. In some embodiments, the AB specifically binds to human CD166 or cynomolgus monkey CD166 with a dissociation constant of less than 1 nM. In some embodiments, the mammalian CD166 is a human CD166. In some embodiments, the mammalian CD166 is a cynomolgus CD166. In some embodiments, the AB has one or more of the following characteristics: (a) the AB specifically binds to human CD166; and (b) the AB specifically binds to human CD166 and cynomolgus monkey CD166. In some embodiments, the AB has one or more of the following characteristics: (a) the AB specifically binds human CD166 and cynomolgus monkey CD166; (b) the AB inhibits binding of mammalian CD6 to mammalian CD166; (c) the AB inhibits binding of human CD6 to human CD166; and (d) the AB inhibits binding of cynomolgus monkey CD6 to cynomolgus monkey CD166.

In some embodiments, the AB blocks the ability of a natural ligand or receptor to bind to the mammalian CD166 with an EC50 less than or equal to 5 nM, less than or equal to 10 nM, less than or equal to 50 nM, less than or equal to 100 nM, less than or equal to 500 nM, and/or less than or equal to 1000 nM. In some embodiments, the AB blocks the ability of mammalian CD6 to bind to the mammalian CD166 with an EC50 less than or equal to 5 nM, less than or equal to 10 nM, less than or equal to 50 nM, less than or equal to 100 nM, less than or equal to 500 nM, and/or less than or equal to 1000 nM. In some embodiments, the natural ligand or receptor of CD166 is CD6.

In some embodiments, the AB blocks the ability of a natural ligand to bind to the mammalian CD166 with an EC50 of 5 nM to 1000 nM, 5 nM to 500 nM, 5 nM to 100 nM, 5 nM to 50 nM, 5 nM to 10 nM, 10 nM to 1000 nM, 10 nM to 500 nM, 10 nM to 100 nM, 10 nM to 50 nM, 50 nM to 1000 nM, 50 nM to 500 nM, 50 nM to 100 nM, 100 nM to 1000 nM, 100 nM to 500 nM, 150 nM to 400 nM, 200 nM to 300 nM, 500 nM to 1000 nM. In some embodiments, the AB blocks the ability of mammalian CD6 to bind to the mammalian CD166 with an EC50 of 5 nM to 1000 nM, 5 nM to 500 nM, 5 nM to 100 nM, 5 nM to 50 nM, 5 nM to 10 nM, 10 nM to 1000 nM, 10 nM to 500 nM, 10 nM to 100 nM, 10 nM to 50 nM, 15 nM to 75 nM, 30 nM to 80 nM, 40 nM to 150 nM, 50 nM to 1000 nM, 50 nM to 500 nM, 50 nM to 100 nM, 100 nM to 1000 nM, 100 nM to 500 nM, 150 nM to 400 nM, 200 nM to 300 nM, 500 nM to 1000 nM. In some embodiments, the natural ligand or receptor of CD166 is CD6.

In some embodiments, the AB of the present disclosure inhibits or reduces the growth, proliferation, and/or metastasis of cells expressing mammalian CD166. Without intending to be bound by any theory, the AB of the present disclosure may inhibit or reduce the growth, proliferation, and/or metastasis of cells expressing mammalian CD166 by specifically binding to CD166 and inhibiting, blocking, and/or preventing the binding of a natural ligand or receptor to mammalian CD166. In some embodiments, the natural ligand or receptor of mammalian CD166 is mammalian CD6.

The antibody or antigen-binding fragment thereof of the AA is coupled to a masking moiety (MM), such that coupling of the MM reduces the ability of the antibody or antigen-binding fragment thereof to bind CD166. In some embodiments, the MM is coupled via a sequence that includes a substrate for a protease, for example, a protease that is active in diseased tissue and/or a protease that is co-localized with CD166 at a treatment site in a subject. The activatable anti-CD166 antibodies provided herein, also referred to herein interchangeably as anti-CD166 AAs or CD166 activatable antibodies, are stable in circulation, activated at intended sites of therapy and/or diagnosis but not in normal, e.g., healthy tissue or other tissue not targeted for treatment and/or diagnosis, and, when activated, exhibit binding to CD166 that is at least comparable to the corresponding, unmodified antibody, also referred to herein as the parental antibody.

The disclosure provides antibodies or antigen-binding fragments thereof (AB) that specifically bind mammalian CD166, for use in the AAs. In some embodiments, the antibody includes an antibody or antigen-binding fragment thereof that specifically binds CD166. In some embodiments, the antibody or antigen-binding fragment thereof that binds CD166 is a monoclonal antibody, domain antibody, single chain, Fab fragment, a F(ab′)2 fragment, a scFv, a scAb, a dAb, a single domain heavy chain antibody, or a single domain light chain antibody. In some embodiments, such an antibody or antigen-binding fragment thereof that binds CD166 is a mouse, other rodent, chimeric, humanized or fully human monoclonal antibody.

Accordingly, provided herein are activatable antibodies (AAs) comprising: (1) an antibody or an antigen binding fragment thereof (AB) that specifically binds to mammalian CD166, a masking moiety (MM) coupled to the AB, wherein the MM inhibits the binding of the AB to the mammalian CD166 when the AA is in an uncleaved state, and a cleavable moiety (CM) coupled to the AB, wherein the CM is a polypeptide that functions as a substrate for a protease,

The antibodies in the AAs of the disclosure (the ABs) specifically bind a CD166 target, such as, for example, mammalian CD166, and/or human CD166.

In some embodiments, the AB has a dissociation constant of about 100 nM or less for binding to mammalian CD166. In some embodiments, the AB has a dissociation constant of about 10 nM or less for binding to mammalian CD166. In some embodiments, the AB has a dissociation constant of about 5 nM or less for binding to CD166. In some embodiments, the AB has a dissociation constant of about 1 nM or less for binding to CD166. In some embodiments, the AB has a dissociation constant of about 0.5 nM or less for binding to CD166. In some embodiments, the AB has a dissociation constant of about 0.1 nM or less for binding to CD166. In some embodiments, the AB has a dissociation constant of 0.01 nM to 100 nM, 0.01 nM to 10 nM, 0.01 nM to 5 nM, 0.01 nM to 1 nM, 0.01 to 0.5 nM, 0.01 nm to 0.1 nM, 0.01 nm to 0.05 nM, 0.05 nM to 100 nM, 0.05 nM to 10 nM, 0.05 nM to 5 nM, 0.05 nM to 1 nM, 0.05 to 0.5 nM, 0.05 nm to 0.1 nM, 0.1 nM to 100 nM, 0.1 nM to 10 nM, 0.1 nM to 5 nM, 0.1 nM to 1 nM, 0.1 to 0.5 nM, 0.5 nM to 100 nM, 0.5 nM to 10 nM, 0.5 nM to 5 nM, 0.5 nM to 1 nM, 1 nM to 100 nM, 1 nM to 10 nM, 1 nM to 5 nM, 5 nM to 100 nM, 5 nM to 10 nM, or 10 nM to 100 nM, for binding to mammalian CD166.

In some embodiments, the AA in an uncleaved state specifically binds to mammalian CD166 with a dissociation constant less than or equal to 1 nM, less than or equal to 5 nM, less than or equal to 10 nM, less than or equal to 15 nM, less than or equal to 20 nM, less than or equal to 25 nM, less than or equal to 50 nM, less than or equal to 100 nM, less than or equal to 150 nM, less than or equal to 250 nM, less than or equal to 500 nM, less than or equal to 750 nM, less than or equal to 1000 nM, and 122. /or less than or equal to 2000 nM.

In some embodiments, the AA in an uncleaved state specifically binds to mammalian CD166 with a dissociation constant greater than or equal to 1 nM, greater than or equal to 5 nM, greater than or equal to 10 nM, greater than or equal to 15 nM, greater than or equal to 20 nM, greater than or equal to 25 nM, greater than or equal to 50 nM, greater than or equal to 100 nM, greater than or equal to 150 nM, greater than or equal to 250 nM, greater than or equal to 500 nM, greater than or equal to 750 nM, greater than or equal to 1000 nM, and 122. /or greater than or equal to 2000 nM.

In some embodiments, the AA in an uncleaved state specifically binds to the mammalian CD166 with a dissociation constant in the range of 1 nM to 2000 nM, 1 nM to 1000 nM, 1 nM to 750 nM, 1 nM to 500 nM, 1 nM to 250 nM, 1 nM to 150 nM, 1 nM to 100 nM, 1 nM to 50 nM, 1 nM to 25 nM, 1 nM to 15 nM, 1 nM to 10 nM, 1 nM to 5 nM, 5 nM to 2000 nM, 5 nM to 1000 nM, 5 nM to 750 nM, 5 nM to 500 nM, 5 nM to 250 nM, 5 nM to 150 nM, 5 nM to 100 nM, 5 nM to 50 nM, 5 nM to 25 nM, 5 nM to 15 nM, 5 nM to 10 nM, 10 nM to 2000 nM, 10 nM to 1000 nM, 10 nM to 750 nM, 10 nM to 500 nM, 10 nM to 250 nM, 10 nM to 150 nM, 10 nM to 100 nM, 10 nM to 50 nM, 10 nM to 25 nM, 10 nM to 15 nM, 15 nM to 2000 nM, 15 nM to 1000 nM, 15 nM to 750 nM, 15 nM to 500 nM, 15 nM to 250 nM, 15 nM to 150 nM, 15 nM to 100 nM, 15 nM to 50 nM, 15 nM to 25 nM, 25 nM to 2000 nM, 25 nM to 1000 nM, 25 nM to 750 nM, 25 nM to 500 nM, 25 nM to 250 nM, 25 nM to 150 nM, 25 nM to 100 nM, 25 nM to 50 nM, 50 nM to 2000 nM, 50 nM to 1000 nM, 50 nM to 750 nM, 50 nM to 500 nM, 50 nM to 250 nM, 50 nM to 150 nM, 50 nM to 100 nM, 100 nM to 2000 nM, 100 nM to 1000 nM, 100 nM to 750 nM, 100 nM to 500 nM, 100 nM to 250 nM, 100 nM to 150 nM, 150 nM to 2000 nM, 150 nM to 1000 nM, 150 nM to 750 nM, 150 nM to 500 nM, 150 nM to 250 nM, 250 nM to 2000 nM, 250 nM to 1000 nM, 250 nM to 750 nM, 250 nM to 500 nM, 500 nM to 2000 nM, 500 nM to 1000 nM, 500 nM to 750 nM, 500 nM to 500 nM, 500 nM to 250 nM, 500 nM to 150 nM, 500 nM to 100 nM, 500 nM to 50 nM, 750 nM to 2000 nM, 750 nM to 1000 nM, or 1000 nM to 2000 nM.

In some embodiments, the AA in an activated state specifically binds to mammalian CD166 with a dissociation constant is less than or equal to 0.01 nM, 0.05 nM, 0.1 nM, 0.5 nM, 1 nM, 5 nM, or 10 nM.

In some embodiments, the AA in an activated state specifically binds to mammalian CD166 with a dissociation constant is greater than or equal to 0.01 nM, 0.05 nM, 0.1 nM, 0.5 nM, 1 nM, 5 nM, or 10 nM.

In some embodiments, the AA in an activated state specifically binds to the mammalian CD166 with a dissociation constant in the range of 0.01 nM to 100 nM, 0.01 nM to 10 nM, 0.01 nM to 5 nM, 0.01 nM to 1 nM, 0.01 to 0.5 nM, 0.01 nm to 0.1 nM, 0.01 nm to 0.05 nM, 0.05 nM to 100 nM, 0.05 nM to 10 nM, 0.05 nM to 5 nM, 0.05 nM to 1 nM, 0.05 to 0.5 nM, 0.05 nm to 0.1 nM, 0.1 nM to 100 nM, 0.1 nM to 10 nM, 0.1 nM to 5 nM, 0.1 nM to 1 nM, 0.1 to 0.5 nM, 0.5 nM to 100 nM, 0.5 nM to 10 nM, 0.5 nM to 5 nM, 0.5 nM to 1 nM, 1 nM to 100 nM, 1 nM to 10 nM, 1 nM to 5 nM, 5 nM to 100 nM, 5 nM to 10 nM, or 10 nM to 100 nM.

Exemplary activatable anti-CD166 antibodies of the invention include, for example, activatable antibodies (AAs) that include a heavy chain and a light chain that comprise, that are, or that are derived from, the heavy chain and light chain variable amino acid sequences shown below:

Human αCD166 Heavy Chain
HuCD166_HcC
(SEQ ID NO: 239)
QITLKESGPTLVKPTQTLTLTCTFSGFSLSTYGMGVGWIRQPPGKALEWL
ANIWWSEDKHYSPSLKSRLTITKDTSKNQVVLTITNVDPVDTATYYCVQI
DYGNDYAFTYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
K
Human αCD166 Heavy Chain
HuCD166_HcC Des-HC
(SEQ ID NO: 480)
QITLKESGPTLVKPTQTLTLTCTFSGFSLSTYGMGVGWIRQPPGKALEWL
ANIWWSEDKHYSPSLKSRLTITKDTSKNQVVLTITNVDPVDTATYYCVQI
DYGNDYAFTYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPG
Human aCD166 Light Chain VL domain
HuCD166_Lc1
(SEQ ID NO: 240)
DIVMTQSPLSLPVTPGEPASISCRSSKSLLHSNGITYLYWYLQKPGQSPQ
LLIYQMSNLASGVPDRFSGSGSGTDFTLKISRVEAEDVGVYYCAQNLELP
YTFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

In some embodiments, the serum half-life of the AA is longer than that of the corresponding antibody; e.g., the pK of the AA is longer than that of the corresponding antibody. In some embodiments, the serum half-life of the AA is similar to that of the corresponding antibody. In some embodiments, the serum half-life of the AA is at least 15 days when administered to an organism. In some embodiments, the serum half-life of the AA is at least 12 days when administered to an organism. In some embodiments, the serum half-life of the AA is at least 11 days when administered to an organism. In some embodiments, the serum half-life of the AA is at least 10 days when administered to an organism. In some embodiments, the serum half-life of the AA is at least 9 days when administered to an organism. In some embodiments, the serum half-life of the AA is at least 8 days when administered to an organism. In some embodiments, the serum half-life of the AA is at least 7 days when administered to an organism. In some embodiments, the serum half-life of the AA is at least 6 days when administered to an organism. In some embodiments, the serum half-life of the AA is at least 5 days when administered to an organism. In some embodiments, the serum half-life of the AA is at least 4 days when administered to an organism. In some embodiments, the serum half-life of the AA is at least 3 days when administered to an organism. In some embodiments, the serum half-life of the AA is at least 2 days when administered to an organism. In some embodiments, the serum half-life of the AA is at least 24 hours when administered to an organism. In some embodiments, the serum half-life of the AA is at least 20 hours when administered to an organism. In some embodiments, the serum half-life of the AA is at least 18 hours when administered to an organism. In some embodiments, the serum half-life of the AA is at least 16 hours when administered to an organism. In some embodiments, the serum half-life of the AA is at least 14 hours when administered to an organism. In some embodiments, the serum half-life of the AA is at least 12 hours when administered to an organism. In some embodiments, the serum half-life of the AA is at least 10 hours when administered to an organism. In some embodiments, the serum half-life of the AA is at least 8 hours when administered to an organism. In some embodiments, the serum half-life of the AA is at least 6 hours when administered to an organism. In some embodiments, the serum half-life of the AA is at least 4 hours when administered to an organism. In some embodiments, the serum half-life of the AA is at least 3 hours when administered to an organism.

### Exemplary Activatable Antibodies

In exemplary embodiments, the AAs of the disclosure comprise any one or more of the following sequences:

Human αCD166 Heavy Chain (HuCD166_HcC)-Amino
Acid Sequence
SEQ ID NO: 239
(provided above)
Human αCD166 Heavy Chain (HuCD166_HcC)-Des-HC-
Amino Acid Sequence
SEQ ID NO: 480
(provided above)
Human aCD166 Light Chain VL domain
HuCD166_Lcl
SEQ ID NO: 240
(provided above)
Amino Acid Sequence
Human αCD166 Light Chain (spacer-MM-LP1-CM-LP2-Ab)
[spacer (SEQ ID NO: 305)] [huCD166Lcl_7614.6_3001
(SEQ ID NO: 314)]
SEQ ID NO: 246
[QGQSGQG][LCHPAVLSAWESCSSGGGSSGGSAVGLLAPPGGLSGRSD
NHGGSDIVMTQSPLSLPVTPGEPASISCRSSKSLLHSNGITYLYWYLQKP
GQSPQLLIYQMSNLASGVPDRFSGSGSGTDFTLKISRVEAEDVGVYYCAQ
NLELPYTFGQGTKLEIKRTVAAPSVFIFPPSDEQLKSGTASVVCLLNNFY
PREAKVQWKVDNALQSGNSQESVTEQDSKDSTYSLSSTLTLSKADYEKHK
VYACEVTHQGLSSPVTKSFNRGEC]
Amino Acid Sequence
Human aCD166 Light Chain (MM-LP1-CM-LP2-Ab)
huCD166Lcl_7614.6_3001
SEQ ID NO: 314
LCHPAVLSAWESCSSGGGSSGGSAVGLLAPPGGLSGRSDNHGGSDIVMTQ
SPLSLPVTPGEPASISCRSSKSLLHSNGITYLYWYLQKPGQSPQLLIYQM
SNLASGVPDRFSGSGSGTDFTLKISRVEAEDVGVYYCAQNLELPYTFGQG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC
Amino Acid Sequence
Spacer
SEQ ID NO: 305
QGQSGQG
Masking Moiety 7614.6
SEQ ID NO: 222
LCHPAVLSAWESCSS
Cleavable Moiety 3001
SEQ ID NO: 76
AVGLLAPPGGLSGRSDNH
Linking peptide 1 (LP1)
SEQ ID NO: 479
GGGSSGGS
Linking Peptide 2 (LP2)
GGS

In an exemplary embodiment, the AA comprises: (a) an antibody or an antigen binding fragment thereof (AB) that specifically binds to mammalian CD166, wherein the AB comprises a heavy chain comprising an amino acid sequence of SEQ ID NO: 480 and a light chain comprising an amino acid sequence of SEQ ID NO: 240; (b) a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and (c) a cleavable moiety (CM) coupled to the AB, wherein the CM is a polypeptide that functions as a substrate for a protease, and wherein the CM comprises the amino acid sequence of SEQ ID NO: 76.

In an exemplary embodiment, the AA comprises: (a) an antibody or an antigen binding fragment thereof (AB) that specifically binds to mammalian CD166, wherein the AB comprises a heavy chain comprising an amino acid sequence of SEQ ID NO: 480 and a light chain comprising an amino acid sequence of SEQ ID NO: 246, and is conjugated to DM4 via spdb linker (this exemplary conjugated AA is herein referred to as “spacer-7614.6-3001-HcCD166-SPDB-DM4”), also referred to as “Combination 55”. The linker toxin SPDB-DM4 is also known as N-succinimidyl 4-(2-pyridyldithio) butanoate-N2′-deacetyl-N2′-(4-mercapto-4-methyl-1-oxopentyl)-maytansine.

In another exemplary embodiment, the AA comprises: (a) an antibody or an antigen binding fragment thereof (AB) that specifically binds to mammalian CD166, wherein the AB comprises a heavy chain comprising an amino acid sequence of SEQ ID NO: 480 and a light chain comprising an amino acid sequence of SEQ ID NO: 314, and is further conjugated to DM4 via spdb linker this exemplary conjugated AA is herein referred to as “7614.6-3001-HcCD166-SPDB-DM4”, also referred to as “Combination 60”).

### Masking Moieties (MM)

The activatable anti-CD166 antibodies described herein overcome a limitation of antibody therapeutics, particularly antibody therapeutics that are known to be toxic to at least some degree in vivo. Target-mediated toxicity constitutes a major limitation for the development of therapeutic antibodies. The activatable anti-CD166 antibodies provided herein are designed to address the toxicity associated with the inhibition of the target in normal tissues by traditional therapeutic antibodies. These activatable anti-CD166 antibodies remain masked until proteolytically activated at the site of disease. Starting with an anti-CD166 antibody as a parental therapeutic antibody, the activatable anti-CD166 antibodies of the invention were engineered by coupling the antibody to an inhibitory mask (masking moiety, MM) through a linker that incorporates a protease substrate (CM).

Accordingly, the activatable anti-CD166 antibodies provided herein include a masking moiety (MM). In some embodiments, the MM is an amino acid sequence that is coupled or otherwise attached to the anti-CD166 antibody and is positioned within the activatable anti-CD166 antibody construct such that the MM reduces the ability of the anti-CD166 antibody to specifically bind CD166. Suitable masking moieties are identified using any of a variety of known techniques. For example, peptide masking moieties are identified using the methods described in PCT Publication No. WO 2009/025846 by Daugherty et al., the contents of which are hereby incorporated by reference in their entirety.

In some embodiments, in the presence of CD166, the MM reduces the ability of the AB to bind CD166 by at least 90% when the CM is uncleaved, as compared to when the CM is cleaved when assayed in vitro using a target displacement assay such as, for example, the assay described in PCT Publication No. WO 2010/081173, the contents of which are hereby incorporated by reference in their entirety.

In some embodiments, the MM is a polypeptide of about 2 to 40 amino acids in length. In some embodiments, the MM is a polypeptide of up to about 40 amino acids in length.

In some embodiments, the MM polypeptide sequence is different from that of CD166. In some embodiments, the MM polypeptide sequence is no more than 50% identical to any natural binding partner of the AB. In some embodiments, the MM polypeptide sequence is different from that of CD166 and is no more than 40%, 30%, 25%, 20%, 15%, or 10% identical to any natural binding partner of the AB.

In one exemplary embodiment, the AAs provided herein comprise an MM, whose amino acid sequence is set forth:

Masking Moiety 7614.6
(SEQ ID NO: 222)
LCHPAVLSAWESCSS

When the AB is modified with a MM and is in the presence of the target, specific binding of the AB to its target is reduced or inhibited, as compared to the specific binding of the AB not modified with an MM or the specific binding of the parental AB to the target.

The K_{d} of the AB modified with a MM towards the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 25-50, 50-250, 100-1,000, 100-10,000, 100-100,000, 100-1,000,000, 100-10,000,000, 500-2,500, 1,000-10,000, 1,000-100,000, 1,000-1,000,000, 1000-10,000,000, 2,500-5,000, 5,000-50,000, 10,000-100,000, 10,000-1,000,000, 10,000-10,000,000, 50,000-5,000,000, 100,000-1,000,000, or 100,000-10,000,000 times greater than the K_{d} of the AB not modified with an MM or of the parental AB towards the target. Conversely, the binding affinity of the AB modified with a MM towards the target is at least 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 25-50, 50-250, 100-1,000, 100-10,000, 100-100,000, 100-1,000,000, 100-10,000,000, 500-2,500, 1,000-10,000, 1,000-100,000, 1,000-1,000,000, 1000-10,000,000, 2,500-5,000, 5,000-50,000, 10,000-100,000, 10,000-1,000,000, 10,000-10,000,000, 50,000-5,000,000, 100,000-1,000,000, or 100,000-10,000,000 times lower than the binding affinity of the AB not modified with an MM or of the parental AB towards the target.

In some embodiments, the coupling of the MM to the AB reduces the ability of the AB to bind CD166 such that the dissociation constant (K_{d}) of the AB when coupled to the MM towards CD166 is at least two times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least five times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least 10 times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least 20 times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least 40 times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least 100 times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least 1000 times greater than the K_{d} of the AB when not coupled to the MM towards CD166.

In some embodiments, the coupling of the MM to the AB reduces the ability of the AB to bind CD166 such that the dissociation constant (K_{d}) of the AB when coupled to the MM towards CD166 is at least 10,000 times greater than the K_{d} of the AB when not coupled to the MM towards CD166.

The dissociation constant (K_{d}) of the MM towards the AB is generally greater than the K_{d} of the AB towards the target. The K_{d} of the MM towards the AB can be at least 5, 10, 25, 50, 100, 250, 500, 1,000, 2,500, 5,000, 10,000, 100,000, 1,000,000 or even 10,000,000 times greater than the K_{d} of the AB towards the target. Conversely, the binding affinity of the MM towards the AB is generally lower than the binding affinity of the AB towards the target. The binding affinity of MM towards the AB can be at least 5, 10, 25, 50, 100, 250, 500, 1,000, 2,500, 5,000, 10,000, 100,000, 1,000,000 or even 10,000,000 times lower than the binding affinity of the AB towards the target.

In some embodiments, the dissociation constant (Kd) of the MM towards the AB is approximately equal to the Kd of the AB towards the target. In some embodiments, the dissociation constant (Kd) of the MM towards the AB is no more than the dissociation constant of the AB towards the target.

In some embodiments, the dissociation constant (Kd) of the MM towards the AB is less than the dissociation constant of the AB towards the target.

In some embodiments, the dissociation constant (Kd) of the MM towards the AB is greater than the dissociation constant of the AB towards the target.

In some embodiments, the MM has a Kd for binding to the AB that is no more than the Kd for binding of the AB to the target.

In some embodiments, the MM has a Kd for binding to the AB that is less than the Kd for binding of the AB to the target.

In some embodiments, the MM has a Kd for binding to the AB that is approximately equal to the Kd for binding of the AB to the target.

In some embodiments, the MM has a Kd for binding to the AB that is no less than the Kd for binding of the AB to the target.

In some embodiments, the MM has a Kd for binding to the AB that is greater than the Kd for binding of the AB to the target.

In some embodiments, the dissociation constant (K_{d}) of the MM towards the AB is no more than 2, 3, 4, 5, 10, 25, 50, 100, 250, 500, 1,000, 2,500, 5,000, 10,000, 50,000, 100,000, 500,000, 1,000,000, 5,000,000, 10,000,000, 50,000,000 times or greater, or between 1-5, 5-10, 10-100, 10-1,000, 10-10,000, 10-100,000, 10-1,000,000, 10-10,000,000, 25-50, 50-250, 100-1,000, 100-10,000, 100-100,000, 100-1,000,000, 100-10,000,000, 25-500, 500-2,500, 1,000-10,000, 1,000-100,000, 1,000-1,000,000, 1000-10,000,000, 2,500-5,000, 5,000-50,000, 10,000-100,000, 10,000-1,000,000, 10,000-10,000,000, 50,000-5,000,000, 100,000-1,000,000, or 100,000-10,000,000 fold greater than the Kd for binding of the AB to the target. In some embodiments, the MM has a Kd for binding to the AB that is between 1-5, 2-5, 2-10, 5-10, 5-20, 5-50, 5-100, 10-100, 10-1,000, 20-100, 20-1000, or 100-1,000-fold greater than the Kd for binding of the AB to the target.

In some embodiments, the MM has an affinity for binding to the AB that is less than the affinity of binding of the AB to the target.

In some embodiments, the MM has an affinity for binding to the AB that is no more than the affinity of binding of the AB to the target.

In some embodiments, the MM has an affinity for binding to the AB that is approximately equal of the affinity of binding of the AB to the target.

In some embodiments, the MM has an affinity for binding to the AB that is no less than the affinity of binding of the AB to the target.

In some embodiments, the MM has an affinity for binding to the AB that is greater than the affinity of binding of the AB to the target.

In some embodiments, the MM has an affinity for binding to the AB that is 2, 3, 4, 5, 10, 25, 50, 100, 250, 500, or 1,000 less than the affinity of binding of the AB to the target. In some embodiments, the MM has an affinity for binding to the AB that is between 1-5, 2-5, 2-10, 5-10, 5-20, 5-25, 5-50, 5-100, 10-100, 10-1,000, 20-100, 20-1000, 25-250, 50-500, or 100-1,000 fold less than the affinity of binding of the AB to the target. In some embodiments, the MM has an affinity for binding to the AB that is 2 to 20-fold less than the affinity of binding of the AB to the target. In some embodiments, a MM not covalently linked to the AB and at equimolar concentration to the AB does not inhibit the binding of the AB to the target.

When the AB is modified with a MM and is in the presence of the target specific binding of the AB to its target is reduced or inhibited, as compared to the specific binding of the AB not modified with an MM or the specific binding of the parental AB to the target. When compared to the binding of the AB not modified with an MM or the binding of the parental AB to the target the AB's ability to bind the target when modified with an MM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or more when measured in vivo or in an in vitro assay.

The MM inhibits the binding of the AB to the target. The MM binds the antigen binding domain of the AB and inhibits binding of the AB to the target. The MM can sterically inhibit the binding of the AB to the target. The MM can allosterically inhibit the binding of the AB to its target. In these embodiments when the AB is modified by or coupled to a MM and in the presence of target there is no binding or substantially no binding of the AB to the target, or no more than 0.001%, 0.01%, 0.1%, 1%, 2%, 3%, 4%, 5%, 6%, 7%, 8%, 9%, 10%, 15%, 20%, 25%, 30%, 35%, 40%, or 50% binding of the AB to the target, as compared to the binding of the AB not modified with an MM, the parental AB, or the AB not coupled to an MM to the target, for at least 2, 4, 6, 8, 12, 28, 24, 30, 36, 48, 60, 72, 84, or 96 hours, or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

When an AB is coupled to or modified by a MM, the MM ‘masks’ reduces or otherwise inhibits the specific binding of the AB to the target. When an AB is coupled to or modified by a MM, such coupling or modification can effect a structural change that reduces or inhibits the ability of the AB to specifically bind its target.

An AB coupled to or modified with an MM can be represented by the following formulae (in order from an amino (N) terminal region to carboxyl (C) terminal region:

(MM)-(AB)

(AB)-(MM)

(MM)-L-(AB)

(AB)-L-(MM)

where MM is a masking moiety, the AB is an antibody or antibody fragment thereof, and the L is a linker. In many embodiments, it may be desirable to insert one or more linkers, e.g., flexible linkers, into the composition so as to provide for flexibility.

In certain embodiments, the MM is not a natural binding partner of the AB. In some embodiments, the MM contains no or substantially no homology to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% similar to any natural binding partner of the AB. In some embodiments, the MM is no more than 5%, 10%, 15%, 20%, 25%, 30%, 35%, 40%, 45%, 50%, 55%, 60%, 65%, 70%, 75%, or 80% identical to any natural binding partner of the AB. In some embodiments, the MM is no more than 25% identical to any natural binding partner of the AB. In some embodiments, the MM is no more than 50% identical to any natural binding partner of the AB. In some embodiments, the MM is no more than 20% identical to any natural binding partner of the AB. In some embodiments, the MM is no more than 10% identical to any natural binding partner of the AB.

### Cleavable Moieties (CM)

The activatable anti-CD166 antibodies provided herein include a cleavable moiety (CM). In some embodiments, the CM includes an amino acid sequence that is a substrate for a protease, usually an extracellular protease. Suitable substrates can be identified using any of a variety of known techniques. For example, peptide substrates are identified using the methods described in U.S. Pat. No. 7,666,817 by Daugherty et al.; in U.S. Pat. No. 8,563,269 by Stagliano et al.; and in PCT Publication No. WO 2014/026136 by La Porte et al., the contents of each of which are hereby incorporated by reference in their entirety. (See also Boulware et al. “Evolutionary optimization of peptide substrates for proteases that exhibit rapid hydrolysis kinetics.” Biotechnol Bioeng. 106.3 (2010): 339-46).

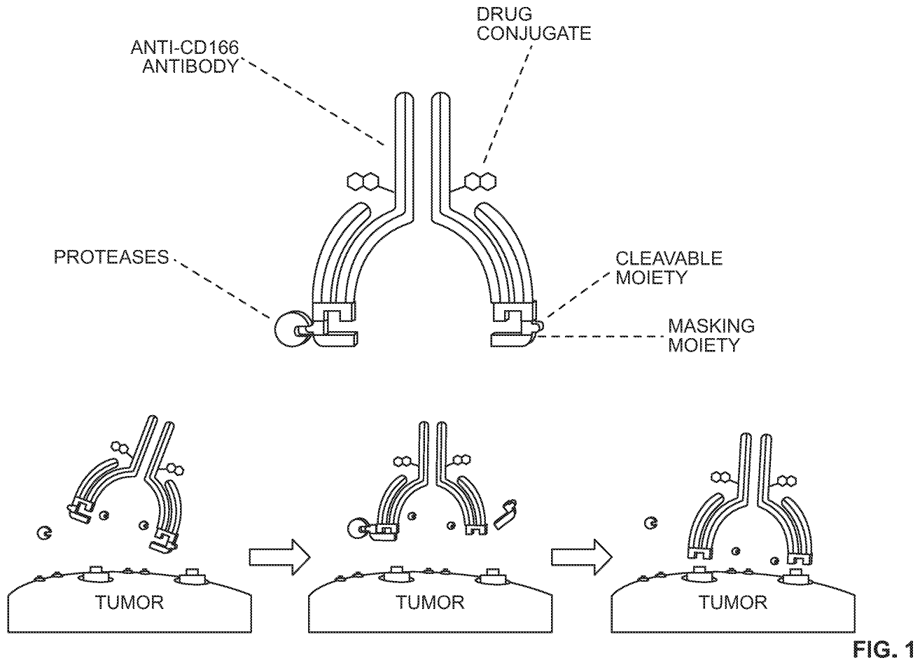

In some embodiments, the protease that cleaves the CM is active, e.g., up-regulated or otherwise unregulated, in diseased tissue, and the protease cleaves the CM in the AA when the AA is exposed to the protease. In some embodiments, the protease is co-localized with CD166 in a tissue, and the protease cleaves the CM in the AA when the AA is exposed to the protease. FIG. 1 depicts activatable anti-CD166 antibody drug conjugates being preferentially activated in the tumor microenvironment, where tumor-specific proteases are present.

In some embodiments, the AAs include an AB that is modified by an MM and also includes one or more cleavable moieties (CM). Such AAs exhibit activatable/switchable binding, to the AB's target. AAs generally include an antibody or antibody fragment (AB), modified by or coupled to a masking moiety (MM) and a modifiable or cleavable moiety (CM). In some embodiments, the CM contains an amino acid sequence that serves as a substrate for at least one protease.

In some embodiments, the CM is a polypeptide of up to 15 amino acids in length.

In some embodiments, the CM is a polypeptide that includes a first cleavable moiety (CM1) that is a substrate for at least one matrix metalloprotease (MMP) and a second cleavable moiety (CM2) that is a substrate for at least one serine protease (SP). In some embodiments, each of the CM1 substrate sequence and the CM2 substrate sequence of the CM1-CM2 substrate is independently a polypeptide of up to 15 amino acids in length.

In some embodiments, the CM is a CM1-CM2 substrate whose amino acid sequence is set forth:

Cleavable Moiety 3001 (Substrate 3001)
(SEQ ID NO: 76)
AVGLLAPPGGLSGRSDNH

The elements of the AAs are arranged so that the MM and CM are positioned such that in a cleaved (or relatively active) state and in the presence of a target, the AB binds a target while the AA is in an uncleaved (or relatively inactive) state in the presence of the target, specific binding of the AB to its target is reduced or inhibited. The specific binding of the AB to its target can be reduced due to the inhibition or masking of the AB's ability to specifically bind its target by the MM.

The K_{d} of the AB modified with a MM and a CM towards the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 25-50, 50-250, 100-1,000, 100-10,000, 100-100,000, 100-1,000,000, 100-10,000,000, 25-500, 500-2,500, 1,000-10,000, 1,000-100,000, 1,000-1,000,000, 1000-10,000,000, 2,500-5,000, 5,000-50,000, 10,000-100,000, 10,000-1,000,000, 10,000-10,000,000, 50,000-5,000,000, 100,000-1,000,000, or 100,000-10,000,000 times greater than the K_{d} of the AB not modified with an MM and a CM or of the parental AB towards the target. Conversely, the binding affinity of the AB modified with a MM and a CM towards the target is at least 5, 10, 25, 50, 100, 250, 500, 1,000, 2,500, 5,000, 10,000, 50,000, 100,000, 500,000, 1,000,000, 5,000,000, 10,000,000, 50,000,000 or greater, or between 5-10, 10-100, 10-1,000, 10-10,000, 10-100,000, 10-1,000,000, 10-10,000,000, 25-50, 50-250, 100-1,000, 100-10,000, 100-100,000, 100-1,000,000, 100-10,000,000, 25-500, 500-2,500, 1,000-10,000, 1,000-100,000, 1,000-1,000,000, 1000-10,000,000, 2,500-5,000, 5,000-50,000, 10,000-100,000, 10,000-1,000,000, 10,000-10,000,000, 50,000-5,000,000, 100,000-1,000,000, or 100,000-10,000,000 times lower than the binding affinity of the AB not modified with an MM and a CM or of the parental AB towards the target.

When the AB is modified with a MM and a CM and is in the presence of the target but not in the presence of a modifying agent (for example at least one protease), specific binding of the AB to its target is reduced or inhibited, as compared to the specific binding of the AB not modified with an MM and a CM or of the parental AB to the target. When compared to the binding of the parental AB or the binding of an AB not modified with an MM and a CM to its target, the AB's ability to bind the target when modified with an MM and a CM can be reduced by at least 50%, 60%, 70%, 80%, 90%, 92%, 93%, 94%, 95%, 96%, 97%, 98%, 99% and even 100% for at least 2, 4, 6, 8, 12, 28, 24, 30, 36, 48, 60, 72, 84, or 96 hours or 5, 10, 15, 30, 45, 60, 90, 120, 150, or 180 days, or 1, 2, 3, 4, 5, 6, 7, 8, 9, 10, 11, or 12 months or longer when measured in vivo or in an in vitro assay.

As used herein, the term “cleaved state” refers to the condition of the AAs following modification of the CM by at least one protease. The term “uncleaved state”, as used herein, refers to the condition of the AAs in the absence of cleavage of the CM by a protease. As discussed above, the term “activatable antibodies” is used herein to refer to an AA in both its uncleaved (native) state, as well as in its cleaved state. It will be apparent to the ordinarily skilled artisan that in some embodiments a cleaved AA may lack an MM due to cleavage of the CM by protease, resulting in release of at least the MM (e.g., where the MM is not joined to the AAs by a covalent bond (e.g., a disulfide bond between cysteine residues).

By activatable or switchable is meant that the AA exhibits a first level of binding to a target when the AA is in a inhibited, masked or uncleaved state (i.e., a first conformation), and a second level of binding to the target in the uninhibited, unmasked and/or cleaved state (i.e., a second conformation), where the second level of target binding is greater than the first level of binding. In general, the access of target to the AB of the AA is greater in the presence of a cleaving agent capable of cleaving the CM, i.e., a protease, than in the absence of such a cleaving agent. Thus, when the AA is in the uncleaved state, the AB is inhibited from target binding and can be masked from target binding (i.e., the first conformation is such the AB cannot bind the target), and in the cleaved state the AB is not inhibited or is unmasked to target binding.

The CM and AB of the AAs are selected so that the AB represents a binding moiety for a given target, and the CM represents a substrate for a protease. In some embodiments, the protease is co-localized with the target at a treatment site or diagnostic site in a subject. As used herein, co-localized refers to being at the same site or relatively close nearby. In some embodiments, a protease cleaves a CM yielding an activated antibody that binds to a target located nearby the cleavage site. The AAs disclosed herein find particular use where, for example, a protease capable of cleaving a site in the CM, i.e., a protease, is present at relatively higher levels in target-containing tissue of a treatment site or diagnostic site than in tissue of non-treatment sites (for example in healthy tissue). In some embodiments, a CM of the disclosure is also cleaved by one or more other proteases. In some embodiments, it is the one or more other proteases that is co-localized with the target and that is responsible for cleavage of the CM in vivo.

In some embodiments AAs provide for reduced toxicity and/or adverse side effects that could otherwise result from binding of the AB at non-treatment sites if the AB were not masked or otherwise inhibited from binding to the target.

In general, an AA can be designed by selecting an AB of interest and constructing the remainder of the AA so that, when conformationally constrained, the MM provides for masking of the AB or reduction of binding of the AB to its target. Structural design criteria can be to be taken into account to provide for this functional feature.

AAs exhibiting a switchable phenotype of a desired dynamic range for target binding in an inhibited versus an uninhibited conformation are provided. Dynamic range generally refers to a ratio of (a) a maximum detected level of a parameter under a first set of conditions to (b) a minimum detected value of that parameter under a second set of conditions. For example, in the context of an activatable antibody, the dynamic range refers to the ratio of (a) a maximum detected level of target protein binding to an AA in the presence of at least one protease capable of cleaving the CM of the AAs to (b) a minimum detected level of target protein binding to an AA in the absence of the protease. The dynamic range of an AA can be calculated as the ratio of the dissociation constant of an AA cleaving agent (e.g., enzyme) treatment to the dissociation constant of the AAs cleaving agent treatment. The greater the dynamic range of an activatable antibody, the better the switchable phenotype of the activatable antibody. AAs having relatively higher dynamic range values (e.g., greater than 1) exhibit more desirable switching phenotypes such that target protein binding by the AAs occurs to a greater extent (e.g., predominantly occurs) in the presence of a cleaving agent (e.g., enzyme) capable of cleaving the CM of the AAs than in the absence of a cleaving agent.

The CM is specifically cleaved by at least one protease at a rate of about 0.001-1500×10⁴ M⁻¹S⁻¹ or at least 0.001, 0.005, 0.01, 0.05, 0.1, 0.5, 1, 2.5, 5, 7.5, 10, 15, 20, 25, 50, 75, 100, 125, 150, 200, 250, 500, 750, 1000, 1250, or 1500×10⁴ M⁻¹S⁻¹. In some embodiments, the CM is specifically cleaved at a rate of about 100,000 M⁻¹S⁻¹. In some embodiments, the CM is specifically cleaved at a rate from about 1×10E2 to about 1×10E6 M⁻¹S⁻¹ (i.e., from about 1×10² to about 1×10⁶ M⁻¹S⁻¹).

For specific cleavage by an enzyme, contact between the enzyme and CM is made. When the AA comprising an AB coupled to a MM and a CM is in the presence of target and sufficient enzyme activity, the CM can be cleaved. Sufficient enzyme activity can refer to the ability of the enzyme to make contact with the CM and effect cleavage. It can readily be envisioned that an enzyme may be in the vicinity of the CM but unable to cleave because of other cellular factors or protein modification of the enzyme.

### Structural Configurations of the Activatable Antibodies

The AAs of the present disclosure can be provided in a variety of structural configurations. Exemplary formulae for AAs are provided below. It is specifically contemplated that the N- to C-terminal order of the AB, MM and CM may be reversed within an activatable antibody. It is also specifically contemplated that the CM and MM may overlap in amino acid sequence, e.g., such that the CM is contained within the MM.

For example, AAs can be represented by the following formula (in order from an amino (N) terminal region to carboxyl (C) terminal region:

(MM)-(CM)-(AB)

(AB)-(CM)-(MM)

where MM is a masking moiety, CM is a cleavable moiety, and AB is an antibody or fragment thereof. It should be noted that although MM and CM are indicated as distinct components in the formulae above, in all exemplary embodiments (including formulae) disclosed herein it is contemplated that the amino acid sequences of the MM and the CM could overlap, e.g., such that the CM is completely or partially contained within the MM. In addition, the formulae above provide for additional amino acid sequences that may be positioned N-terminal or C-terminal to the AAs elements.

In many embodiments it may be desirable to insert one or more linkers, e.g., flexible linkers, into the AA construct so as to provide for flexibility at one or more of the MM-CM junction, the CM-AB junction, or both. For example, the AB, MM, and/or CM may not contain a sufficient number of residues (e.g., Gly, Ser, Asp, Asn, especially Gly and Ser, particularly Gly) to provide the desired flexibility. As such, the switchable phenotype of such AA constructs may benefit from introduction of one or more amino acids to provide for a flexible linker. In addition, as described below, where the AA is provided as a conformationally constrained construct, a flexible linker can be operably inserted to facilitate formation and maintenance of a cyclic structure in the uncleaved activatable antibody.

In some embodiments, the AA comprises a first linking peptide (LP1) and a second linking peptide (LP2), and wherein the AA in the uncleaved state has the structural arrangement from N-terminus to C-terminus as follows: MM-LP1-CM-LP2-AB or AB-LP2-CM-LP1-MM. In some embodiments, the two linking peptides need not be identical to each other.

In some embodiments, at least one of LP1 or LP2 comprises an amino acid sequence selected from the group consisting of (GS)ₙ, (GGS)ₙ, (GSGGS)ₙ (SEQ ID NO: 1) and (GGGS)ₙ (SEQ ID NO: 2), where n is an integer of at least one.

In some embodiments, at least one of LP1 or LP2 comprises an amino acid sequence selected from the group consisting of GGSG (SEQ ID NO: 3), GGSGG (SEQ ID NO: 4), GSGSG (SEQ ID NO: 5), GSGGG (SEQ ID NO: 6), GGGSG (SEQ ID NO: 7), and GSSSG (SEQ ID NO: 8).

In some embodiments, LP1 comprises the amino acid sequence GSSGGSGGSGGSG (SEQ ID NO: 9), GSSGGSGGSGG (SEQ ID NO: 10), GSSGGSGGSGGS (SEQ ID NO: 11), GSSGGSGGSGGSGGGS (SEQ ID NO: 12), GSSGGSGGSG (SEQ ID NO: 13), or GSSGGSGGSGS (SEQ ID NO: 14).

In some embodiments, LP2 comprises the amino acid sequence GSS, GGS, GGGS (SEQ ID NO: 15), GSSGT (SEQ ID NO: 16) or GSSG (SEQ ID NO: 17).

In some embodiments, the AB has a dissociation constant of about 100 nM or less for binding to CD166.

For example, in certain embodiments an AA comprises one of the following formulae (where the formula below represents an amino acid sequence in either N- to C-terminal direction or C- to N-terminal direction):

(MM)-LP1-(CM)-(AB)

(MM)-(CM)-LP2-(AB)

(MM)-LP1-(CM)-LP2-(AB)

wherein MM, CM, and AB are as defined above; wherein LP1 and LP2 are each independently and optionally present or absent, are the same or different flexible linkers that include at least 1 flexible amino acid (e.g., Gly). In addition, the formulae above provide for additional amino acid sequences that may be positioned N-terminal or C-terminal to the AAs elements. Examples include, but are not limited to, targeting moieties (e.g., a ligand for a receptor of a cell present in a target tissue) and serum half-life extending moieties (e.g., polypeptides that bind serum proteins, such as immunoglobulin (e.g., IgG) or serum albumin (e.g., human serum albumin (HAS)).

In some embodiments, the AA is exposed to and cleaved by a protease such that, in the activated or cleaved state, the activated antibody includes a light chain amino acid sequence that includes at least a portion of LP2 and/or CM sequence after the protease has cleaved the CM.

Linkers suitable for use in compositions described herein are generally ones that provide flexibility of the modified AB or the AAs to facilitate the inhibition of the binding of the AB to the target. Such linkers are generally referred to as flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, 7, 8, 9, 10, 11, 12, 13, 14, 15, 16, 17, 18, 19, or 20 amino acids in length.

Exemplary flexible linkers include glycine polymers (G)n, glycine-serine polymers (including, for example: (GS)n, (GSGGS)n (SEQ ID NO: 1) and (GGGS)n (SEQ ID NO: 2), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine and is much less restricted than residues with longer side chains (see Scheraga, Rev. Computational Chem. 11173-142 (1992)). Exemplary flexible linkers include, but are not limited to Gly-Gly-Ser-Gly (SEQ ID NO: 3), Gly-Gly-Ser-Gly-Gly (SEQ ID NO: 4), Gly-Ser-Gly-Ser-Gly (SEQ ID NO: 5), Gly-Ser-Gly-Gly-Gly (SEQ ID NO: 6), Gly-Gly-Gly-Ser-Gly (SEQ ID NO: 7), Gly-Ser-Ser-Ser-Gly (SEQ ID NO: 8), and the like. The ordinarily skilled artisan will recognize that design of an AAs can include linkers that are all or partially flexible, such that the linker can include a flexible linker as well as one or more portions that confer less flexible structure to provide for a desired AAs structure.

In some embodiments, the AA also includes a signal peptide. In some embodiments, the signal peptide is conjugated to the AA via a spacer. In some embodiments, the spacer is conjugated to the AA in the absence of a signal peptide. In some embodiments, the spacer is joined directly to the MM of the activatable antibody. In some embodiments, the spacer is joined directly to the MM of the AA in the structural arrangement from N-terminus to C-terminus of spacer-MM-CM-AB. An example of a spacer joined directly to the N-terminus of MM of the AA is QGQSGQ (SEQ ID NO: 88). Other examples of a spacer joined directly to the N-terminus of MM of the AA include QGQSGQG (SEQ ID NO: 305), QGQSG (SEQ ID NO: 306), QGQS (SEQ ID NO: 307), QGQ, QG, and Q. Other examples of a spacer joined directly to the N-terminus of MM of the AA include GQSGQG (SEQ ID NO: 359), QSGQG (SEQ ID NO: 360), SGQG (SEQ ID NO: 361), GQG, and G. In some embodiments, no spacer is joined to the N-terminus of the MM. In some embodiments, the spacer includes at least the amino acid sequence QGQSGQ (SEQ ID NO: 88). In some embodiments, the spacer includes at least the amino acid sequence QGQSGQG (SEQ ID NO: 305). In some embodiments, the spacer includes at least the amino acid sequence QGQSG (SEQ ID NO: 306). In some embodiments, the spacer includes at least the amino acid sequence QGQS (SEQ ID NO: 307). In some embodiments, the spacer includes at least the amino acid sequence QGQ. In some embodiments, the spacer includes at least the amino acid sequence QG. In some embodiments, the spacer includes at least the amino acid residue Q. In some embodiments, the spacer includes at least the amino acid sequence GQSGQG (SEQ ID NO: 359). In some embodiments, the spacer includes at least the amino acid sequence QSGQG (SEQ ID NO: 360). In some embodiments, the spacer includes at least the amino acid sequence SGQG (SEQ ID NO: 361). In some embodiments, the spacer includes at least the amino acid sequence GQG. In some embodiments, the spacer includes at least the amino acid sequence G. In some embodiments, the spacer is absent.

### Conjugated Activatable Antibodies

The AA compositions and methods provided herein enable the attachment of one or more agents to one or more cysteine residues (e.g. cysteine, lysine) in the AB without compromising the activity (e.g., the masking, activating or binding activity) of the activatable anti-CD166 antibody. In some embodiments, the compositions and methods provided herein enable the attachment of one or more agents to one or more cysteine residues in the AB without reducing or otherwise disturbing one or more disulfide bonds within the MM. The compositions and methods provided herein produce an activatable anti-CD166 antibody that is conjugated to one or more agents, e.g., any of a variety of therapeutic, diagnostic and/or prophylactic agents, for example, in some embodiments, without any of the agent(s) being conjugated to the MM of the activatable anti-CD166 antibody. The compositions and methods provided herein produce conjugated activatable anti-CD166 antibodies in which the MM retains the ability to effectively and efficiently mask the AB of the AA in an uncleaved state. The compositions and methods provided herein produce conjugated activatable anti-CD166 antibodies in which the AA is still activated, i.e., cleaved, in the presence of a protease that can cleave the CM.

In some embodiments, the AAs described herein also include an agent conjugated to the activatable antibody. In some embodiments, the conjugated agent is a therapeutic agent, such as an anti-inflammatory and/or an antineoplastic agent. In such embodiments, the agent is conjugated to a carbohydrate moiety of the activatable antibody, for example, in some embodiments, where the carbohydrate moiety is located outside the antigen-binding region of the antibody or antigen-binding fragment in the activatable antibody. In some embodiments, the agent is conjugated to a sulfhydryl group of the antibody or antigen-binding fragment in the activatable antibody.

In some embodiments, the agent is a cytotoxic agent such as a toxin (e.g., an enzymatically active toxin of bacterial, fungal, plant, or animal origin, or fragments thereof), or a radioactive isotope (i.e., a radioconjugate).

In some embodiments, the agent is a detectable moiety such as, for example, a label or other marker. For example, the agent is or includes a radiolabeled amino acid, one or more biotinyl moieties that can be detected by marked avidin (e.g., streptavidin containing a fluorescent marker or enzymatic activity that can be detected by optical or calorimetric methods), one or more radioisotopes or radionuclides, one or more fluorescent labels, one or more enzymatic labels, and/or one or more chemiluminescent agents. In some embodiments, detectable moieties are attached by spacer molecules.

The disclosure also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate). Suitable cytotoxic agents include, for example, dolastatins and derivatives thereof (e.g. auristatin E, AFP, MMAF, MMAE, MMAD, DMAF, DMAE). For example, the agent is monomethyl auristatin E (MMAE) or monomethyl auristatin D (MMAD). In some embodiments, the agent is an agent selected from the group listed in Table 1. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In an exemplary embodiment, the agent is DM4.

In some embodiments, the agent is linked to the AB using a maleimide caproyl-valine-citrulline linker or a maleimide PEG-valine-citrulline linker. In some embodiments, the agent is linked to the AB using a maleimide caproyl-valine-citrulline linker. In some embodiments, the agent is linked to the AB using a maleimide PEG-valine-citrulline linker In some embodiments, the agent is monomethyl auristatin D (MMAD) linked to the AB using a maleimide PEG-valine-citrulline-para-aminobenzyloxycarbonyl linker, and this linker payload construct is referred to herein as “vc-MMAD.” In some embodiments, the agent is monomethyl auristatin E (MMAE) linked to the AB using a maleimide PEG-valine-citrulline-para-aminobenzyloxycarbonyl linker, and this linker payload construct is referred to herein as “vc-MMAE.” In some embodiments, the agent is linked to the AB using a maleimide PEG-valine-citrulline linker In some embodiments, the agent is monomethyl auristatin D (MMAD) linked to the AB using a maleimide bis-PEG-valine-citrulline-para-aminobenzyloxycarbonyl linker, and this linker payload construct is referred to herein as “PEG2-vc-MMAD.” The structures of vc-MMAD, vc-MMAE, and PEG2-vc-MMAD are shown below:

vc-MMAD:

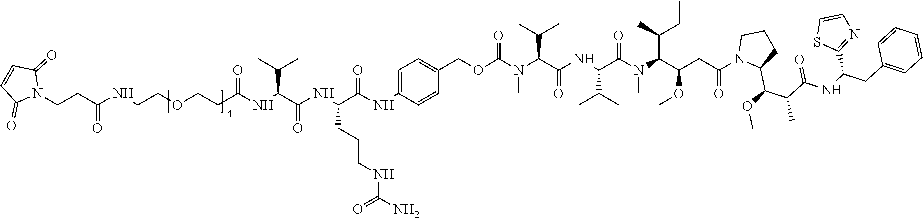

vc-MMAE:

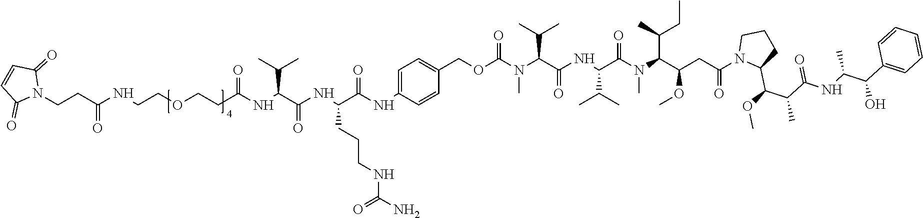

### PEG2-vc-MMAD:

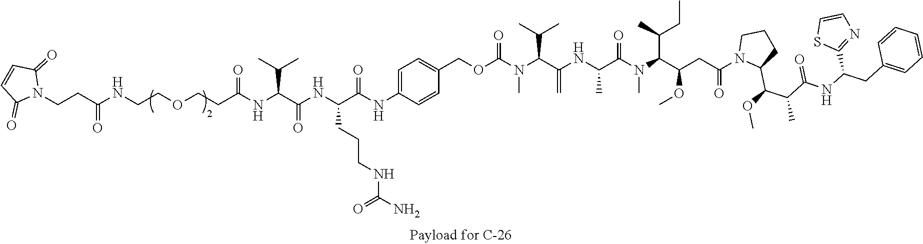

In an exemplary embodiment, the agent is conjugated to the AA via lysine. In an exemplary embodiment an SPDB-DM4 is attached to an activatable antibody through the epsilon-amino group of a lysine on the AA, e.g. The epsilon-amino group of the lysine.

In an exemplary embodiment, the agent is DM4 and the linker-DM is as follows:

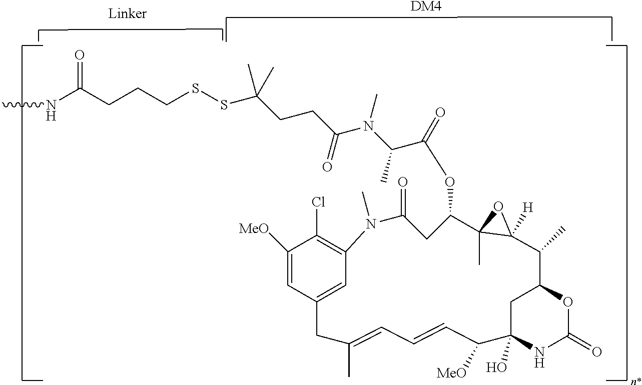

The disclosure also provides conjugated AAs that include an AA linked to monomethyl auristatin D (MMAD) payload, wherein the AA includes an antibody or an antigen binding fragment thereof (AB) that specifically binds to a target, a masking moiety (MM) that inhibits the binding of the AB of the AA in an uncleaved state to the target, and cleavable moiety (CM) coupled to the AB, and the CM is a polypeptide that functions as a substrate for at least one MMP protease.

In some embodiments, the MMAD-conjugated AA can be conjugated using any of several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB.

In some embodiments, the MMAD payload is conjugated to the AB via a linker. In some embodiments, the MMAD payload is conjugated to a cysteine in the AB via a linker. In some embodiments, the MMAD payload is conjugated to a lysine in the AB via a linker. In some embodiments, the MMAD payload is conjugated to another residue of the AB via a linker, such as those residues disclosed herein. In some embodiments, the linker is a thiol-containing linker. In some embodiments, the linker is a cleavable linker. In some embodiments, the linker is a non-cleavable linker. In some embodiments, the linker is selected from the group consisting of the linkers shown in Tables 6 and 7. In some embodiments, the AA and the MMAD payload are linked via a maleimide caproyl-valine-citrulline linker. In some embodiments, the AA and the MMAD payload are linked via a maleimide PEG-valine-citrulline linker. In some embodiments, the AA and the MMAD payload are linked via a maleimide caproyl-valine-citrulline-para-aminobenzyloxycarbonyl linker. In some embodiments, the AA and the MMAD payload are linked via a maleimide PEG-valine-citrulline-para-aminobenzyloxycarbonyl linker. In some embodiments, the MMAD payload is conjugated to the AB using the partial reduction and conjugation technology disclosed herein.

In some embodiments, the polyethylene glycol (PEG) component of a linker of the present disclosure is formed from 2 ethylene glycol monomers, 3 ethylene glycol monomers, 4 ethylene glycol monomers, 5 ethylene glycol monomers, 6 ethylene glycol monomers, 7 ethylene glycol monomers 8 ethylene glycol monomers, 9 ethylene glycol monomers, or at least 10 ethylene glycol monomers. In some embodiments of the present disclosure, the PEG component is a branched polymer. In some embodiments of the present disclosure, the PEG component is an unbranched polymer. In some embodiments, the PEG polymer component is functionalized with an amino group or derivative thereof, a carboxyl group or derivative thereof, or both an amino group or derivative thereof and a carboxyl group or derivative thereof.

In some embodiments, the PEG component of a linker of the present disclosure is an amino-tetra-ethylene glycol-carboxyl group or derivative thereof. In some embodiments, the PEG component of a linker of the present disclosure is an amino-tri-ethylene glycol-carboxyl group or derivative thereof. In some embodiments, the PEG component of a linker of the present disclosure is an amino-di-ethylene glycol-carboxyl group or derivative thereof. In some embodiments, an amino derivative is the formation of an amide bond between the amino group and a carboxyl group to which it is conjugated. In some embodiments, a carboxyl derivative is the formation of an amide bond between the carboxyl group and an amino group to which it is conjugated. In some embodiments, a carboxyl derivative is the formation of an ester bond between the carboxyl group and a hydroxyl group to which it is conjugated.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Table 1 lists some of the exemplary pharmaceutical agents that may be employed in the herein described disclosure but in no way is meant to be an exhaustive list.

TABLE 1
Exemplary Pharmaceutical Agents for Conjugation

CYTOTOXIC AGENTS
Auristatins
Auristatin E
Monomethyl auristatin D (MMAD)
Monomethyl auristatin E (MMAE)
Desmethyl auristatin E (DMAE)
Auristatin F
Monomethyl auristatin F (MMAF)
Desmethyl auristatin F (DMAF)
Auristatin derivatives, e.g., amides thereof
Auristatin tyramine
Auristatin quinoline
Dolastatins
Dolastatin derivatives
Dolastatin 16 DmJ
Dolastatin 16 Dpv
Maytansinoids, e.g. DM-1; DM-4
Maytansinoid derivatives
Duocarmycin
Duocarmycin derivatives
Alpha-amanitin
Anthracyclines
Doxorubicin
Daunorubicin
Bryostatins
Camptothecin
Camptothecin derivatives
7-substituted Camptothecin
10,11-Difluoromethylenedioxycamptothecin
Combretastatins
Debromoaplysiatoxin
Kahalalide-F
Discodermolide
Ecteinascidins
ANTIVIRALS
Acyclovir
Vira A
Symmetrel
ANTIFUNGALS
Nystatin
ADDITIONAL ANTI-NEOPLASTICS
Adriamycin
Cerubidine
Bleomycin
Alkeran
Velban
Oncovin
Fluorouracil
Methotrexate
Thiotepa
Bisantrene
Novantrone
Thioguanine
Procarabizine
Cytarabine
ANTI-BACTERIALS
Aminoglycosides
Streptomycin
Neomycin
Kanamycin
Amikacin
Gentamicin
Tobramycin
Streptomycin B
Spectinomycin
Ampicillin
Sulfanilamide
Polymyxin
Chloramphenicol
Turbostatin
Phenstatins
Hydroxyphenstatin
Spongistatin 5
Spongistatin 7
Halistatin 1
Halistatin 2
Halistatin 3
Modified Bryostatins
Halocomstatins
Pyrrolobenzimidazoles (PBI)
Cibrostatin6
Doxaliform
Anthracyclins analogues
Cemadotin analogue (CemCH2-SH)
Pseudomonas toxin A (PE38) variant
Pseudomonas toxin A (ZZ-PE38) variant
ZJ-101
OSW-1
4-Nitrobenzyloxycarbonyl Derivatives of
O6-Benzylguanine
Topoisomerase inhibitors
Hemiasterlin
Cephalotaxine
Homoharringtonine
Pyrrolobenzodiazepine dimers (PBDs)
Functionalized pyrrolobenzodiazepenes
Calicheamicins
Podophyllotoxins
Taxanes
Vinca alkaloids
CONJUGATABLE DETECTION REAGENTS
Fluorescein and derivatives thereof
Fluorescein isothiocyanate (FITC)
RADIOPHARMACEUTICALS
¹²⁵I
¹³¹I
⁸⁹Zr
¹¹¹In
¹²³I
¹³¹I
⁹⁹mTc
²⁰¹Tl
¹³³Xe
¹¹C
⁶²Cu
¹⁸F
⁶⁸Ga
¹³N
¹⁵O
³⁸K
⁸²Rb
⁹⁹mTc (Technetium)
HEAVY METALS
Barium
Gold
Platinum
ANTI-MYCOPLASMALS
Tylosine
Spectinomycin

Those of ordinary skill in the art will recognize that a large variety of possible moieties can be coupled to the resultant antibodies of the disclosure. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

In some embodiments, the AA is conjugated to one or more equivalents of an agent. In some embodiments, the AA is conjugated to one equivalent of the agent. In some embodiments, the AA is conjugated to two, three, four, five, six, seven, eight, nine, ten, or greater than ten equivalents of the agent. In some embodiments, the AA is part of a mixture of AAs having a homogeneous number of equivalents of conjugated agents. In some embodiments, the AA is part of a mixture of AAs having a heterogeneous number of equivalents of conjugated agents. In some embodiments, the mixture of AAs is such that the average number of agents conjugated to each AA is between zero to one, between one to two, between two and three, between three and four, between four and five, between five and six, between six and seven, between seven and eight, between eight and nine, between nine and ten, and ten and greater. In some embodiments, the mixture of AAs is such that the average number of agents conjugated to each AA is one, two, three, four, five, six, seven, eight, nine, ten, or greater. In some embodiments, there is a mixture of AAs such that the average number of agents conjugated to each AA is between three and four. In some embodiments, there is a mixture of AAs such that such that the average number of agents conjugated to each AA is between 3.4 and 3.8. In some embodiments, there is a mixture of AAs such that such that the average number of agents conjugated to each AA is between 3.4 and 3.6. In some embodiments, the AA comprises one or more site-specific amino acid sequence modifications such that the number of lysine and/or cysteine residues is increased or decreased with respect to the original amino acid sequence of the activatable antibody, thus in some embodiments correspondingly increasing or decreasing the number of agents that can be conjugated to the activatable antibody, or in some embodiments limiting the conjugation of the agents to the AA in a site-specific manner. In some embodiments, the modified AA is modified with one or more non-natural amino acids in a site-specific manner, thus in some embodiments limiting the conjugation of the agents to only the sites of the non-natural amino acids.

### Compositions and Methods to Generate Conjugated Activatable Antibodies

The activatable anti-CD166 antibodies have at least one point of conjugation for an agent (to produce a conjugated AA). In some embodiments, not all possible points of conjugation are used. In some embodiments, some of the natural points of contact are modified or removed to no longer be available for conjugation to an agent. In some embodiments, the one or more points of conjugation are nitrogen atoms, such as the epsilon amino group of lysine.

In some embodiments, the one or more points of conjugation are sulfur atoms involved in disulfide bonds. In some embodiments, the one or more points of conjugation are sulfur atoms involved in interchain disulfide bonds. In some embodiments, the one or more points of conjugation are sulfur atoms involved in interchain sulfide bonds, but not sulfur atoms involved in intrachain disulfide bonds. In some embodiments, the one or more points of conjugation are sulfur atoms of cysteine or other amino acid residues containing a sulfur atom. Such residues may occur naturally in the antibody structure or may be incorporated into the antibody by site-directed mutagenesis, chemical conversion, or mis-incorporation of non-natural amino acids.

Also provided are methods of preparing a conjugate of an activatable anti-CD166 antibody having one or more interchain disulfide bonds in the AB and one or more intrachain disulfide bonds in the MM, and a drug reactive with free thiols is provided. The method generally includes partially reducing interchain disulfide bonds in the AA with a reducing agent, such as, for example, TCEP; and conjugating the drug reactive with free thiols to the partially reduced activatable antibody. As used herein, the term partial reduction refers to situations where an activatable anti-CD166 antibody is contacted with a reducing agent and less than all disulfide bonds, e.g., less than all possible sites of conjugation are reduced. In some embodiments, less than 99%, 98%, 97%, 96%, 95%, 90%, 85%, 80%, 75%, 70%, 65%, 60%, 55%, 50%, 45%, 40%, 35%, 30%, 25%, 20%, 15%, 10% or less than 5% of all possible sites of conjugation are reduced.

In yet other embodiments, a method of reducing and conjugating an agent, e.g., a drug, to an activatable anti-CD166 antibody resulting in selectivity in the placement of the agent is provided. The method generally includes partially reducing the activatable anti-CD166 antibody with a reducing agent such that any conjugation sites in the masking moiety or other non-AB portion of the AA are not reduced, and conjugating the agent to interchain thiols in the AB. The conjugation site(s) are selected so as to allow desired placement of an agent to allow conjugation to occur at a desired site. The reducing agent is, for example, TCEP. The reduction reaction conditions such as, for example, the ratio of reducing agent to activatable antibody, the length of incubation, the temperature during the incubation, the pH of the reducing reaction solution, etc., are determined by identifying the conditions that produce a conjugated AA in which the MM retains the ability to effectively and efficiently mask the AB of the AA in an uncleaved state. The ratio of reduction agent to activatable anti-CD166 antibody will vary depending on the activatable antibody. In some embodiments, the ratio of reducing agent to activatable anti-CD166 antibody will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

In some embodiments, a method of reducing interchain disulfide bonds in the AB of an activatable anti-CD166 antibody and conjugating an agent, e.g., a thiol-containing agent such as a drug, to the resulting interchain thiols to selectively locate agent(s) on the AB is provided. The method generally includes partially reducing the AB with a reducing agent to form at least two interchain thiols without forming all possible interchain thiols in the activatable antibody; and conjugating the agent to the interchain thiols of the partially reduced AB. For example, the AB of the AA is partially reduced for about 1 hour at about 37° C. at a desired ratio of reducing agent:activatable antibody. In some embodiments, the ratio of reducing agent to AA will be in a range from about 20:1 to 1:1, from about 10:1 to 1:1, from about 9:1 to 1:1, from about 8:1 to 1:1, from about 7:1 to 1:1, from about 6:1 to 1:1, from about 5:1 to 1:1, from about 4:1 to 1:1, from about 3:1 to 1:1, from about 2:1 to 1:1, from about 20:1 to 1:1.5, from about 10:1 to 1:1.5, from about 9:1 to 1:1.5, from about 8:1 to 1:1.5, from about 7:1 to 1:1.5, from about 6:1 to 1:1.5, from about 5:1 to 1:1.5, from about 4:1 to 1:1.5, from about 3:1 to 1:1.5, from about 2:1 to 1:1.5, from about 1.5:1 to 1:1.5, or from about 1:1 to 1:1.5. In some embodiments, the ratio is in a range of from about 5:1 to 1:1. In some embodiments, the ratio is in a range of from about 5:1 to 1.5:1. In some embodiments, the ratio is in a range of from about 4:1 to 1:1. In some embodiments, the ratio is in a range from about 4:1 to 1.5:1. In some embodiments, the ratio is in a range from about 8:1 to about 1:1. In some embodiments, the ratio is in a range of from about 2.5:1 to 1:1.

The thiol-containing reagent can be, for example, cysteine or N-acetyl cysteine. The reducing agent can be, for example, TCEP. In some embodiments, the reduced AA can be purified prior to conjugation, using for example, column chromatography, dialysis, or diafiltration. Alternatively, the reduced antibody is not purified after partial reduction and prior to conjugation.

The invention also provides partially reduced activatable anti-CD166 antibodies in which at least one interchain disulfide bond in the AA has been reduced with a reducing agent without disturbing any intrachain disulfide bonds in the activatable antibody, wherein the AA includes an antibody or an antigen binding fragment thereof (AB) that specifically binds to CD166, a masking moiety (MM) that inhibits the binding of the AB of the AA in an uncleaved state to the CD166 target, and a cleavable moiety (CM) coupled to the AB, wherein the CM is a polypeptide that functions as a substrate for a protease. In some embodiments the MM is coupled to the AB via the CM. In some embodiments, one or more intrachain disulfide bond(s) of the AA is not disturbed by the reducing agent. In some embodiments, one or more intrachain disulfide bond(s) of the MM within the AA is not disturbed by the reducing agent. In some embodiments, the AA in the uncleaved state has the structural arrangement from N-terminus to C-terminus as follows: MM-CM-AB or AB-CM-MM. In some embodiments, reducing agent is TCEP.

The disclosure also provides partially reduced AAs in which at least one interchain disulfide bond in the AA has been reduced with a reducing agent without disturbing any intrachain disulfide bonds in the activatable antibody, wherein the AA includes an antibody or an antigen binding fragment thereof (AB) that specifically binds to the target, e.g., CD166, a masking moiety (MM) that inhibits the binding of the AB of the AA in an uncleaved state to the target, and a cleavable moiety (CM) coupled to the AB, wherein the CM is a polypeptide that functions as a substrate for at least one protease. In some embodiments, the MM is coupled to the AB via the CM. In some embodiments, one or more intrachain disulfide bond(s) of the AA is not disturbed by the reducing agent. In some embodiments, one or more intrachain disulfide bond(s) of the MM within the AA is not disturbed by the reducing agent. In some embodiments, the AA in the uncleaved state has the structural arrangement from N-terminus to C-terminus as follows: MM-CM-AB or AB-CM-MM. In some embodiments, reducing agent is TCEP.

In yet other embodiments, a method of reducing and conjugating an agent, e.g., a drug, to an activatable anti-CD166 antibody resulting in selectivity in the placement of the agent by providing an activatable anti-CD166 antibody with a defined number and positions of lysine and/or cysteine residues. In some embodiments, the defined number of lysine and/or cysteine residues is higher or lower than the number of corresponding residues in the amino acid sequence of the parent antibody or activatable antibody. In some embodiments, the defined number of lysine and/or cysteine residues may result in a defined number of agent equivalents that can be conjugated to the anti-CD166 antibody or activatable anti-CD166 antibody. In some embodiments, the defined number of lysine and/or cysteine residues may result in a defined number of agent equivalents that can be conjugated to the anti-CD166 antibody or activatable anti-CD166 antibody in a site-specific manner. In some embodiments, the modified A is modified with one or more non-natural amino acids in a site-specific manner, thus in some embodiments limiting the conjugation of the agents to only the sites of the non-natural amino acids. In some embodiments, the anti-CD166 antibody or activatable anti-CD166 antibody with a defined number and positions of lysine and/or cysteine residues may be partially reduced with a reducing agent as discussed herein such that any conjugation sites in the masking moiety or other non-AB portion of the AA are not reduced, and conjugating the agent to interchain thiols in the AB.

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. In some embodiments, the binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present disclosure, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133:1335-2549 (1984); Jansen et al., Immunological Reviews 62:185-216 (1982); and Vitetta et al., Science 238:1098 (1987).

In some embodiments, in addition to the compositions and methods provided herein, the conjugated AA can also be modified for site-specific conjugation through modified amino acid sequences inserted or otherwise included in the AA sequence. These modified amino acid sequences are designed to allow for controlled placement and/or dosage of the conjugated agent within a conjugated activatable antibody. For example, the AA can be engineered to include cysteine substitutions at positions on light and heavy chains that provide reactive thiol groups and do not negatively impact protein folding and assembly, nor alter antigen binding. In some embodiments, the AA can be engineered to include or otherwise introduce one or more non-natural amino acid residues within the AA to provide suitable sites for conjugation. In some embodiments, the AA can be engineered to include or otherwise introduce enzymatically activatable peptide sequences within the AA sequence.

Suitable linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. In some embodiments, suitable linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC. Additional linkers include, but are not limited to, SMCC ((succinimidyl 4-(N-maleimidomethyl)cyclohexane-1-carboxylate), sulfo-SMCC (sulfosuccinimidyl 4-(N-maleimidomethyl)cyclohexane-1-carboxylate), SPDB (N-succinimidyl-4-(2-pyridyldithio) butanoate), or sulfo-SPDB (N-succinimidyl-4-(2-pyridyldithio)-2-sulfo butanoate).

The linkers described above contain components that have different attributes, thus leading to conjugates with differing physio-chemical properties. For example, sulfo-NHS esters of alkyl carboxylates are more stable than sulfo-NHS esters of aromatic carboxylates. NETS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone. In an exemplary embodiment the linker is SPDB. In another exemplary embodiment, the linker is SPDB agent is DM4.

In some embodiments, the linkers are cleavable. In some embodiments, the linkers are non-cleavable. In some embodiments, two or more linkers are present. The two or more linkers are all the same, i.e., cleavable or non-cleavable, or the two or more linkers are different, i.e., at least one cleavable and at least one non-cleavable.

The present disclosure utilizes several methods for attaching agents to ABs: (a) attachment to the carbohydrate moieties of the AB, or (b) attachment to sulfhydryl groups of the AB, or (c) attachment to amino groups of the AB, or (d) attachment to carboxylate groups of the AB. According to the disclosure, ABs may be covalently attached to an agent through an intermediate linker having at least two reactive groups, one to react with AB and one to react with the agent. The linker, which may include any compatible organic compound, can be chosen such that the reaction with AB (or agent) does not adversely affect AB reactivity and selectivity. Furthermore, the attachment of linker to agent might not destroy the activity of the agent. Suitable linkers for reaction with oxidized antibodies or oxidized antibody fragments include those containing an amine selected from the group consisting of primary amine, secondary amine, hydrazine, hydrazide, hydroxylamine, phenylhydrazine, semicarbazide and thiosemicarbazide groups. Such reactive functional groups may exist as part of the structure of the linker or may be introduced by suitable chemical modification of linkers not containing such groups.

According to the present disclosure, suitable linkers for attachment to reduced ABs include those having certain reactive groups capable of reaction with a sulfhydryl group of a reduced antibody or fragment. Such reactive groups include, but are not limited to: reactive haloalkyl groups (including, for example, haloacetyl groups), p-mercuribenzoate groups and groups capable of Michael-type addition reactions (including, for example, maleimides and groups of the type described by Mitra and Lawton, 1979, J. Amer. Chem. Soc. 101: 3097-3110).

According to the present disclosure, suitable linkers for attachment to neither oxidized nor reduced Abs include those having certain functional groups capable of reaction with the primary amino groups present in unmodified lysine residues in the Ab. Such reactive groups include, but are not limited to, NHS carboxylic or carbonic esters, sulfo-NHS carboxylic or carbonic esters, 4-nitrophenyl carboxylic or carbonic esters, pentafluorophenyl carboxylic or carbonic esters, acyl imidazoles, isocyanates, and isothiocyanates.

According to the present disclosure, suitable linkers for attachment to neither oxidized nor reduced Abs include those having certain functional groups capable of reaction with the carboxylic acid groups present in aspartate or glutamate residues in the Ab, which have been activated with suitable reagents. Suitable activating reagents include EDC, with or without added NHS or sulfo-NHS, and other dehydrating agents utilized for carboxamide formation. In these instances, the functional groups present in the suitable linkers would include primary and secondary amines, hydrazines, hydroxylamines, and hydrazides.

The agent may be attached to the linker before or after the linker is attached to the AB. In certain applications it may be desirable to first produce an AB-linker intermediate in which the linker is free of an associated agent. Depending upon the particular application, a specific agent may then be covalently attached to the linker. In some embodiments, the AB is first attached to the MM, CM and associated linkers and then attached to the linker for conjugation purposes.

Branched Linkers: In specific embodiments, branched linkers that have multiple sites for attachment of agents are utilized. For multiple site linkers, a single covalent attachment to an AB would result in an AB-linker intermediate capable of binding an agent at a number of sites. The sites may be aldehyde or sulfhydryl groups or any chemical site to which agents can be attached.

In some embodiments, higher specific activity (or higher ratio of agents to AB) can be achieved by attachment of a single site linker at a plurality of sites on the AB. This plurality of sites may be introduced into the AB by either of two methods. First, one may generate multiple aldehyde groups and/or sulfhydryl groups in the same AB. Second, one may attach to an aldehyde or sulfhydryl of the AB a “branched linker” having multiple functional sites for subsequent attachment to linkers. The functional sites of the branched linker or multiple site linker may be aldehyde or sulfhydryl groups, or may be any chemical site to which linkers may be attached. Still higher specific activities may be obtained by combining these two approaches, that is, attaching multiple site linkers at several sites on the AB.

Cleavable Linkers: Peptide linkers that are susceptible to cleavage by enzymes of the complement system, such as but not limited to u-plasminogen activator, tissue plasminogen activator, trypsin, plasmin, or another enzyme having proteolytic activity may be used in one embodiment of the present disclosure. According to one method of the present disclosure, an agent is attached via a linker susceptible to cleavage by complement. The antibody is selected from a class that can activate complement. The antibody-agent conjugate, thus, activates the complement cascade and releases the agent at the target site. According to another method of the present disclosure, an agent is attached via a linker susceptible to cleavage by enzymes having a proteolytic activity such as a u-plasminogen activator, a tissue plasminogen activator, plasmin, or trypsin. These cleavable linkers are useful in conjugated AAs that include an extracellular toxin, e.g., by way of non-limiting example, any of the extracellular toxins shown in Table 1.

Non-limiting examples of cleavable linker sequences are provided in Table 2.

TABLE 2
Exemplary Linker Sequences for Conjugation

| Types of Cleavable Sequences | Amino Acid Sequence |
|---|---|
| Plasmin cleavable sequences | |
| Pro-urokinase | PRFKIIGG (SEQ ID NO: 89) |
| | PRFRIIGG (SEQ ID NO: 90) |
| TGFβ | SSRHRRALD (SEQ ID NO: 91) |
| Plasminogen | RKSSIIIRMRDVVL (SEQ ID NO: 92) |
| Staphylokinase | SSSFDKGKYKKGDDA (SEQ ID NO: 93) |
| | SSSFDKGKYKRGDDA (SEQ ID NO: 94) |
| Factor Xa cleavable sequences | IEGR (SEQ ID NO: 95) |
| | IDGR (SEQ ID NO: 96) |
| | GGSIDGR (SEQ ID NO: 97) |
| MMP cleavable sequences | |
| Gelatinase A | PLGLWA (SEQ ID NO: 98) |
| Collagenase cleavable sequences | |
| Calf skin collagen (α1(I) chain) | GPQGIAGQ (SEQ ID NO: 99) |
| Calf skin collagen (α2(I) chain) | GPQGLLGA (SEQ ID NO: 100) |
| Bovine cartilage collagen (α1(II) chain) | GIAGQ (SEQ ID NO: 101) |
| Human liver collagen (α1(III) chain) | GPLGIAGI (SEQ ID NO: 102) |
| Human α₂M | GPEGLRVG (SEQ ID NO: 103) |
| Human PZP | YGAGLGVV (SEQ ID NO: 104) |
| | AGLGVVER (SEQ ID NO: 105) |
| | AGLGISST (SEQ ID NO: 106) |
| Rat α₁M | EPQALAMS (SEQ ID NO: 107) |
| | QALAMSAI (SEQ ID NO: 108) |
| Rat α₂M | AAYHLVSQ (SEQ ID NO: 109) |
| | MDAFLESS (SEQ ID NO: 110) |
| Rat α₁I₃(2J) | ESLPVVAV (SEQ ID NO: 111) |
| Rat α₁I₃(27J) | SAPAVESE (SEQ ID NO: 112) |
| Human fibroblast collagenase | DVAQFVLT (SEQ ID NO: 113) |
| (autolytic cleavages) | VAQFVLTE (SEQ ID NO: 114) |
| | AQFVLTEG (SEQ ID NO: 115) |
| | PVQPIGPQ (SEQ ID NO: 116) |

In addition, agents may be attached via disulfide bonds (for example, the disulfide bonds on a cysteine molecule) to the AB. Since many tumors naturally release high levels of glutathione (a reducing agent) this can reduce the disulfide bonds with subsequent release of the agent at the site of delivery. In some embodiments, the reducing agent that would modify a CM would also modify the linker of the conjugated activatable antibody.

Spacers and Cleavable Elements: In some embodiments, it may be necessary to construct the linker in such a way as to optimize the spacing between the agent and the AB of the activatable antibody. This may be accomplished by use of a linker of the general structure:

W—(CH₂)n-Q

wherein
W is either —NH—CH₂— or —CH₂—;
Q is an amino acid, peptide; and
n is an integer from 0 to 20.

In some embodiments, the linker may comprise a spacer element and a cleavable element. The spacer element serves to position the cleavable element away from the core of the AB such that the cleavable element is more accessible to the enzyme responsible for cleavage. Certain of the branched linkers described above may serve as spacer elements.

Throughout this discussion, it should be understood that the attachment of linker to agent (or of spacer element to cleavable element, or cleavable element to agent) need not be particular mode of attachment or reaction. Any reaction providing a product of suitable stability and biological compatibility is acceptable.

Serum Complement and Selection of Linkers: According to one method of the present disclosure, when release of an agent is desired, an AB that is an antibody of a class that can activate complement is used. The resulting conjugate retains both the ability to bind antigen and activate the complement cascade. Thus, according to this embodiment of the present disclosure, an agent is joined to one end of the cleavable linker or cleavable element and the other end of the linker group is attached to a specific site on the AB. For example, if the agent has a hydroxy group or an amino group, it may be attached to the carboxy terminus of a peptide, amino acid or other suitably chosen linker via an ester or amide bond, respectively. For example, such agents may be attached to the linker peptide via a carbodimide reaction. If the agent contains functional groups that would interfere with attachment to the linker, these interfering functional groups can be blocked before attachment and deblocked once the product conjugate or intermediate is made. The opposite or amino terminus of the linker is then used either directly or after further modification for binding to an AB that is capable of activating complement.

Linkers (or spacer elements of linkers) may be of any desired length, one end of which can be covalently attached to specific sites on the AB of the activatable antibody. The other end of the linker or spacer element may be attached to an amino acid or peptide linker.

Thus, when these conjugates bind to antigen in the presence of complement the amide or ester bond that attaches the agent to the linker will be cleaved, resulting in release of the agent in its active form. These conjugates, when administered to a subject, will accomplish delivery and release of the agent at the target site, and are particularly effective for the in vivo delivery of pharmaceutical agents, antibiotics, antimetabolites, antiproliferative agents and the like as presented in but not limited to those in Table 1.

Linkers for Release without Complement Activation: In yet another application of targeted delivery, release of the agent without complement activation is desired since activation of the complement cascade will ultimately lyse the target cell. Hence, this approach is useful when delivery and release of the agent should be accomplished without killing the target cell. Such is the goal when delivery of cell mediators such as hormones, enzymes, corticosteroids, neurotransmitters, genes or enzymes to target cells is desired. These conjugates may be prepared by attaching the agent to an AB that is not capable of activating complement via a linker that is mildly susceptible to cleavage by serum proteases. When this conjugate is administered to an individual, antigen-antibody complexes will form quickly whereas cleavage of the agent will occur slowly, thus resulting in release of the compound at the target site.

Biochemical Cross Linkers: In some embodiments, the AA may be conjugated to one or more therapeutic agents using certain biochemical cross-linkers. Cross-linking reagents form molecular bridges that tie together functional groups of two different molecules. To link two different proteins in a step-wise manner, hetero-bifunctional cross-linkers can be used that eliminate unwanted homopolymer formation.

Peptidyl linkers cleavable by lysosomal proteases are also useful, for example, Val-Cit, Val-Ala or other dipeptides. In addition, acid-labile linkers cleavable in the low-pH environment of the lysosome may be used, for example: bis-sialyl ether. Other suitable linkers include cathepsin-labile substrates, particularly those that show optimal function at an acidic pH.

Exemplary hetero-bifunctional cross-linkers are referenced in Table 3.

TABLE 3
Exemplary Hetero-Bifunctional Cross Linkers
HETERO-BIFUNCTIONAL CROSS-LINKERS

| | | | Spacer Arm |
|---|---|---|---|
| | | | Length after |
| | | Advantages and | cross-linking |
| Linker | Reactive Toward | Applications | (Angstroms) |
| SMPT | Primary amines | Greater stability | 11.2 Å |
| | Sulfhydryls | | |
| SPDP | Primary amines | Thiolation | 6.8 Å |
| | Sulfhydryls | Cleavable cross-linking | |
| LC-SPDP | Primary amines | Extended spacer arm | 15.6 Å |
| | Sulfhydryls | | |
| Sulfo-LC-SPDP | Primary amines | Extender spacer arm | 15.6 Å |
| | Sulfhydryls | Water-soluble | |
| SMCC | Primary amines | Stable maleimide reactive | 11.6 Å |
| | | group | |
| | Sulfhydryls | Enzyme-antibody | |
| | | conjugation | |
| | | Hapten-carrier protein | |
| | | conjugation | |
| Sulfo-SMCC | Primary amines | Stable maleimide reactive | 11.6 Å |
| | | group | |
| | Sulfhydryls | Water-soluble | |
| | | Enzyme-antibody | |
| | | conjugation | |
| MBS | Primary amines | Enzyme-antibody | 9.9 Å |
| | | conjugation | |
| | Sulfhydryls | Hapten-carrier protein | |
| | | conjugation | |
| Sulfo-MBS | Primary amines | Water-soluble | 9.9 Å |
| | Sulfhydryls | | |
| SIAB | Primary amines | Enzyme-antibody | 10.6 Å |
| | | conjugation | |
| | Sulfhydryls | | |
| Sulfo-SIAB | Primary amines | Water-soluble | 10.6 Å |
| | Sulfhydryls | | |
| SMPB | Primary amines | Extended spacer arm | 14.5 Å |
| | Sulfhydryls | Enzyme-antibody | |
| | | conjugation | |
| Sulfo-SMPB | Primary amines | Extended spacer arm | 14.5 Å |
| | Sulfhydryls | Water-soluble | |
| EDE/Sulfo-NHS | Primary amines | Hapten-Carrier | 0 |
| | | conjugation | |
| | Carboxyl groups | | |
| ABH | Carbohydrates | Reacts with sugar groups | 11.9 Å |
| | Nonselective | | |

Non-Cleavable Linkers or Direct Attachment: In some embodiments of the disclosure, the conjugate may be designed so that the agent is delivered to the target but not released. This may be accomplished by attaching an agent to an AB either directly or via a non-cleavable linker.

These non-cleavable linkers may include amino acids, peptides, D-amino acids or other organic compounds that may be modified to include functional groups that can subsequently be utilized in attachment to ABs by the methods described herein. A—general formula for such an organic linker could be

W—(CH₂)n-Q

wherein
W is either —NH—CH₂— or —CH₂—;
Q is an amino acid, peptide; and
n is an integer from 0 to 20.

Non-Cleavable Conjugates: In some embodiments, a compound may be attached to ABs that do not activate complement. When using ABs that are incapable of complement activation, this attachment may be accomplished using linkers that are susceptible to cleavage by activated complement or using linkers that are not susceptible to cleavage by activated complement.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present disclosure can be conjugated to the liposomes as described in Martin et al., J. Biol. Chem., 257: 286-288 (1982) via a disulfide-interchange reaction.

### Multispecific Activatable Antibodies

_In some embodiments, the activatable anti-CD166 antibody and/or conjugated activatable anti-CD166 antibody is monospecific.

The disclosure also provides multispecific anti-CD166 activatable antibodies. Accordingly, in some embodiments, the activatable anti-CD166 antibody and/or conjugated activatable anti-CD166 antibody is multispecific, e.g., by way of non-limiting example, bispecific or trifunctional. In some embodiments, the activatable anti-CD166 antibody and/or conjugated activatable anti-CD166 antibody is formulated as part of a pro-Bispecific T Cell Engager (BITE) molecule. In some embodiments, the activatable anti-CD166 antibody and/or conjugated activatable anti-CD166 antibody is formulated as part of a pro-Chimeric Antigen Receptor (CAR) modified T cell or other engineered receptor.

In some embodiments, the AA or antigen-binding fragment thereof is incorporated in a multispecific AA or antigen-binding fragment thereof, where at least one arm of the multispecific AA specifically binds CD166. In some embodiments, the AA or antigen-binding fragment thereof is incorporated in a bispecific antibody or antigen-binding fragment thereof, where at least one arm of the bispecific AA specifically binds CD166.

The multispecific AAs provided herein are multispecific antibodies that recognize CD166 and at least one or more different antigens or epitopes and that include at least one masking moiety (MM) linked to at least one antigen- or epitope-binding domain of the multispecific antibody such that coupling of the MM reduces the ability of the antigen- or epitope-binding domain to bind its target. In some embodiments, the MM is coupled to the antigen- or epitope-binding domain of the multispecific antibody via a cleavable moiety (CM) that functions as a substrate for at least one protease. The activatable multispecific antibodies provided herein are stable in circulation, activated at intended sites of therapy and/or diagnosis but not in normal, i.e., healthy tissue, and, when activated, exhibit binding to a target that is at least comparable to the corresponding, unmodified multispecific antibody.

In some embodiments, the multispecific AAs are designed to engage immune effector cells, also referred to herein as immune-effector cell engaging multispecific activatable antibodies. In some embodiments, the multispecific AAs are designed to engage leukocytes, also referred to herein as leukocyte engaging multispecific activatable antibodies. In some embodiments, the multispecific AAs are designed to engage T cells, also referred to herein as T-cell engaging multispecific activatable antibodies. In some embodiments, the multispecific AAs engage a surface antigen on a leukocyte, such as on a T cell, on a natural killer (NK) cell, on a myeloid mononuclear cell, on a macrophage, and/or on another immune effector cell. In some embodiments, the immune effector cell is a leukocyte. In some embodiments, the immune effector cell is a T cell. In some embodiments, the immune effector cell is a NK cell. In some embodiments, the immune effector cell is a mononuclear cell, such as a myeloid mononuclear cell. In some embodiments, the multispecific AAs are designed to bind or otherwise interact with more than one target and/or more than one epitope, also referred to herein as multi-antigen targeting activatable antibodies. As used herein, the terms “target” and “antigen” are used interchangeably.

In some embodiments, immune effector cell engaging multispecific AAs of the disclosure include a targeting antibody or antigen-binding fragment thereof that binds CD166 and an immune effector cell engaging antibody or antigen-binding portion thereof, where at least one of the targeting antibody or antigen-binding fragment thereof and/or the immune effector cell engaging antibody or antigen-binding portion thereof is masked. In some embodiments, the immune effector cell engaging antibody or antigen binding fragment thereof includes a first antibody or antigen-binding fragment thereof (AB1) that binds a first, immune effector cell engaging target, where the AB1 is attached to a masking moiety (MM1) such that coupling of the MM1 reduces the ability of the AB1 to bind the first target. In some embodiments, the targeting antibody or antigen-binding fragment thereof includes a second antibody or fragment thereof that includes a second antibody or antigen-binding fragment thereof (AB2) that binds CD166, where the AB2 is attached to a masking moiety (MM2) such that coupling of the MM2 reduces the ability of the AB2 to bind CD166. In some embodiments, the immune effector cell engaging antibody or antigen binding fragment thereof includes a first antibody or antigen-binding fragment thereof (AB1) that binds a first, immune effector cell engaging target, where the AB1 is attached to a masking moiety (MM1) such that coupling of the MM1 reduces the ability of the AB1 to bind the first target, and the targeting antibody or antigen-binding fragment thereof includes a second antibody or fragment thereof that includes a second antibody or antigen-binding fragment thereof (AB2) that binds CD166, where the AB2 is attached to a masking moiety (MM2) such that coupling of the MM2 reduces the ability of the AB2 to bind CD166. In some embodiments, the non-immune effector cell engaging antibody is a cancer targeting antibody. In some embodiments the non-immune cell effector antibody is an IgG. In some embodiments the immune effector cell engaging antibody is a scFv. In some embodiments the CD166-targeting antibody (e.g., non-immune cell effector antibody) is an IgG and the immune effector cell engaging antibody is a scFv. In some embodiments, the immune effector cell is a leukocyte. In some embodiments, the immune effector cell is a T cell. In some embodiments, the immune effector cell is a NK cell. In some embodiments, the immune effector cell is a myeloid mononuclear cell.

In some embodiments, T-cell engaging multispecific AAs of the disclosure include a CD166-targeting antibody or antigen-binding fragment thereof and a T-cell engaging antibody or antigen-binding portion thereof, where at least one of the CD166-targeting antibody or antigen-binding fragment thereof and/or the T-cell engaging antibody or antigen-binding portion thereof is masked. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target. In some embodiments, the targeting antibody or antigen-binding fragment thereof includes a second antibody or fragment thereof that includes a second antibody or antigen-binding fragment thereof (AB2) that binds CD166, where the AB2 is attached to a masking moiety (MM2) such that coupling of the MM2 reduces the ability of the AB2 to bind CD166. In some embodiments, the T-cell engaging antibody or antigen binding fragment thereof includes a first antibody or antigen-binding fragment thereof (AB1) that binds a first, T-cell engaging target, where the AB1 is attached to a masking moiety (MM1) such that coupling of the MM1 reduces the ability of the AB1 to bind the first target, and the targeting antibody or antigen-binding fragment thereof includes a second antibody or fragment thereof that includes a second antibody or antigen-binding fragment thereof (AB2) that binds CD166, where the AB2 is attached to a masking moiety (MM2) such that coupling of the MM2 reduces the ability of the AB2 to bind CD166.

In some embodiments of an immune effector cell engaging multispecific activatable antibody, one antigen is CD166, and another antigen is typically a stimulatory or inhibitory receptor present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA. In some embodiments, the antigen is a stimulatory receptor present on the surface of a T cell or NK cell; examples of such stimulatory receptors include, but are not limited to, CD3, CD27, CD28, CD137 (also referred to as 4-1BB), GITR, HVEM, ICOS, NKG2D, and OX40. In some embodiments, the antigen is an inhibitory receptor present on the surface of a T-cell; examples of such inhibitory receptors include, but are not limited to, BTLA, CTLA-4, LAG3, PD-1, TIGIT, TIM3, and NK-expressed KIRs. The antibody domain conferring specificity to the T-cell surface antigen may also be substituted by a ligand or ligand domain that binds to a T-cell receptor, a NK-cell receptor, a macrophage receptor, and/or other immune effector cell receptor, such as, but not limited to, B7-1, B7-2, B7H3, PDL1, PDL2, or TNFSF9.

In some embodiments, the T-cell engaging multispecific AA includes an anti-CD3 epsilon (CD3ε, also referred to herein as CD3e and CD3) scFv and a targeting antibody or antigen-binding fragment thereof, where at least one of the anti-CD3ε scFv and/or the targeting antibody or antigen-binding portion thereof is masked. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε. In some embodiments, the targeting antibody or antigen-binding fragment thereof includes a second antibody or fragment thereof that includes a second antibody or antigen-binding fragment thereof (AB2) that binds CD166, where the AB2 is attached to a masking moiety (MM2) such that coupling of the MM2 reduces the ability of the AB2 to bind CD166. In some embodiments, the CD3ε scFv includes a first antibody or antigen-binding fragment thereof (AB1) that binds CD3ε, where the AB1 is attached to a masking moiety (MM1) such that coupling of the MM1 reduces the ability of the AB1 to bind CD3ε, and the targeting antibody or antigen-binding fragment thereof includes a second antibody or fragment thereof that includes a second antibody or antigen-binding fragment thereof (AB2) that binds CD166, where the AB2 is attached to a masking moiety (MM2) such that coupling of the MM2 reduces the ability of the AB2 to bind CD166.

In some embodiments, the multi-antigen targeting antibodies and/or multi-antigen targeting AAs include at least a first antibody or antigen-binding fragment thereof that binds a first target and/or first epitope and a second antibody or antigen-binding fragment thereof that binds a second target and/or a second epitope. In some embodiments, the multi-antigen targeting antibodies and/or multi-antigen targeting AAs bind two or more different targets. In some embodiments, the multi-antigen targeting antibodies and/or multi-antigen targeting AAs bind two or more different epitopes on the same target. In some embodiments, the multi-antigen targeting antibodies and/or multi-antigen targeting AAs bind a combination of two or more different targets and two or more different epitopes on the same target.

In some embodiments, a multispecific AA comprising an IgG has the IgG variable domains masked. In some embodiments, a multispecific AA comprising a scFv has the scFv domains masked. In some embodiments, a multispecific AA has both IgG variable domains and scFv domains, where at least one of the IgG variable domains is coupled to a masking moiety. In some embodiments, a multispecific AA has both IgG variable domains and scFv domains, where at least one of the scFv domains is coupled to a masking moiety. In some embodiments, a multispecific AA has both IgG variable domains and scFv domains, where at least one of the IgG variable domains is coupled to a masking moiety and at least one of the scFv domains is coupled to a masking moiety. In some embodiments, a multispecific AA has both IgG variable domains and scFv domains, where each of the IgG variable domains and the scFv domains is coupled to its own masking moiety. In some embodiments, one antibody domain of a multispecific AA has specificity for a target antigen and another antibody domain has specificity for a T-cell surface antigen. In some embodiments, one antibody domain of a multispecific AA has specificity for a target antigen and another antibody domain has specificity for another target antigen. In some embodiments, one antibody domain of a multispecific AA has specificity for an epitope of a target antigen and another antibody domain has specificity for another epitope of the target antigen.

In a multispecific activatable antibody, a scFv can be fused to the carboxyl terminus of the heavy chain of an IgG activatable antibody, to the carboxyl terminus of the light chain of an IgG activatable antibody, or to the carboxyl termini of both the heavy and light chains of an IgG activatable antibody. In a multispecific activatable antibody, a scFv can be fused to the amino terminus of the heavy chain of an IgG activatable antibody, to the amino terminus of the light chain of an IgG activatable antibody, or to the amino termini of both the heavy and light chains of an IgG activatable antibody. In a multispecific activatable antibody, a scFv can be fused to any combination of one or more carboxyl termini and one or more amino termini of an IgG activatable antibody. In some embodiments, a masking moiety (MM) linked to a cleavable moiety (CM) is attached to and masks an antigen binding domain of the IgG. In some embodiments, a masking moiety (MM) linked to a cleavable moiety (CM) is attached to and masks an antigen binding domain of at least one scFv. In some embodiments, a masking moiety (MM) linked to a cleavable moiety (CM) is attached to and masks an antigen binding domain of an IgG and a masking moiety (MM) linked to a cleavable moiety (CM) is attached to and masks an antigen binding domain of at least one scFv.

The disclosure provides examples of multispecific AA structures which include, but are not limited to, the following: (VL-CL)₂:(VH-CH1-CH2-CH3-L4-VH*-L3-VL*-L2-CM-L1-MM)₂; (VL-CL)₂:(VH-CH1-CH2-CH3-L4-VL*-L3-VH*-L2-CM-L1-MM)₂; (MM-L1-CM-L2-VL-CL)₂:(VH-CH1-CH2-CH3-L4-VH*-L3-VL*)₂; (MM-L1-CM-L2-VL-CL)₂:(VH-CH1-CH2-CH3-L4-VL*-L3-VH*)₂; (VL-CL)₂:(MM-L1-CM-L2-VL*-L3-VH*-L4-VH-CH1-CH2-CH3)₂; (VL-CL)₂:(MM-L1-CM-L2-VH*-L3-VL*-L4-VH-CH1-CH2-CH3)₂; (MM-L1-CM-L2-VL-CL)₂:(VL*-L3-VH*-L4-VH-CH1-CH2-CH3)₂; (MM-L1-CM-L2-VL-CL)₂: (VH*-L3-VL*-L4-VH-CH1-CH2-CH3)₂; (VL-CL-L4-VH*-L3-VL*-L2-CM-L1-MM)₂:(VH-CH1-CH2-CH3)₂; (VL-CL-L4-VL*-L3-VH*-L2-CM-L1-MM)₂:(VH-CH1-CH2-CH3)₂; (MM-L1-CM-L2-VL*-L3-VH*-L4-VL-CL)₂:(VH-CH1-CH2-CH3)₂; (MM-L1-CM-L2-VH*-L3-VL*-L4-VL-CL)₂:(VH-CH1-CH2-CH3)₂; (VL-CL-L4-VH*-L3-VL*-L2-CM-L1-MM)₂:(MM-L1-CM-L2-VL*-L3-VH*-L4-VH-CH1-CH2-CH3)₂; (VL-CL-L4-VH*-L3-VL*-L2-CM-L1-MM)₂:(MM-L1-CM-L2-VH*-L3-VL*-L4-VH-CH1-CH2-CH3)₂; (VL-CL-L4-VL*-L3-VH*-L2-CM-L1-MM)₂:(MM-L1-CM-L2-VL*-L3-VH*-L4-VH-CH1-CH2-CH3)₂; (VL-CL-L4-VL*-L3-VH*-L2-CM-L1-MM)₂:(MM-L1-CM-L2-VH*-L3-VL*-L4-VH-CH1-CH2-CH3)₂; (VL-CL-L4-VH*-L3-VL*)₂: (MM-L1-CM-L2-VL*-L3-VH*-L4-VH-CH1-CH2-CH3)₂; (VL-CL-L4-VH*-L3-VL*)₂:(MM-L1-CM-L2-VH*-L3-VL*-L4-VH-CH1-CH2-CH3)₂; (VL-CL-L4-VL*-L3-VH*)₂:(MM-L1-CM-L2-VL*-L3-VH*-L4-VH-CH1-CH2-CH3)₂; (VL-CL-L4-VL*-L3-VH*)₂: (MM-L1-CM-L2-VH*-L3-VL*-L4-VH-CH1-CH2-CH3)₂; (VL-CL-L4-VH*-L3-VL*-L2-CM-L1-MM)₂:(VL*-L3-VH*-L4-VH-CH1-CH2-CH3)₂; (VL-CL-L4-VH*-L3-VL*-L2-CM-L1-MM)₂: (VH*-L3-VL*-L4-VH-CH1-CH2-CH3)₂; (VL-CL-L4-VL*-L3-VH*-L2-CM-L1-MM)₂:(VL*-L3-VH*-L4-VH-CH1-CH2-CH3)₂; or (VL-CL-L4-VL*-L3-VH*-L2-CM-L1-MM)₂: (VH*-L3-VL*-L4-VH-CH1-CH2-CH3)₂, wherein: VL and VH represent the light and heavy variable domains of the first specificity, contained in the IgG; VL* and VH* represent the variable domains of the second specificity, contained in the scFv; L1 is a linker peptide connecting the masking moiety (MM) and the CM (CM); L2 is a linker peptide connecting the CM (CM), and the antibody; L3 is a linker peptide connecting the variable domains of the scFv; L4 is a linker peptide connecting the antibody of the first specificity to the antibody of the second specificity; CL is the light-chain constant domain; and CH1, CH2, CH3 are the heavy chain constant domains. The first and second specificities may be toward any antigen or epitope.

In some embodiments of a T-cell engaging multispecific activatable antibody, one antigen is CD166, and another antigen is typically a stimulatory (also referred to herein as activating) or inhibitory receptor present on the surface of a T-cell, natural killer (NK) cell, myeloid mononuclear cell, macrophage, and/or other immune effector cell, such as, but not limited to, B7-H4, BTLA, CD3, CD4, CD8, CD16a, CD25, CD27, CD28, CD32, CD56, CD137 (also referred to as TNFRSF9), CTLA-4, GITR, HVEM, ICOS, LAG3, NKG2D, OX40, PD-1, TIGIT, TIM3, or VISTA. The antibody domain conferring specificity to the T-cell surface antigen may also be substituted by a ligand or ligand domain that binds to a T-cell receptor, a NK-cell receptor, a macrophage receptor, and/or other immune effector cell receptor.

In some embodiments, the targeting antibody is an anti-CD166 antibody disclosed herein. In some embodiments, the targeting antibody can be in the form an activatable antibody. In some embodiments, the scFv(s) can be in the form of a Pro-scFv (see, e.g., WO 2009/025846, WO 2010/081173).

In some embodiments, the scFv is specific for binding CDR, and comprises or is derived from an antibody or fragment thereof that binds CDR, e.g., CH2527, FN18, H2C, OKT3, 2C11, UCHT1, or V9. In some embodiments, the scFv is specific for binding CTLA-4 (also referred to herein as CTLA and CTLA4).

In some embodiments, the anti-CTLA-4 scFv includes the amino acid sequence:

Anti-CTLA-4 scFv
(SEQ ID NO: 117)
GGGSGGGGSGSGGGSGGGGSGGGEIVLTQSPGTLSLSPGERATLSCRASQ
SVSSSYLAWYQQKPGQAPRLLIYGASSRATGIPDRFSGSGSGTDFTLTIS
RLEPEDFAVYYCQQYGSSPLTFGGGTKVEIKRSGGSTITSYNVYYTKLSS
SGTQVQLVQTGGGVVQPGRSLRLSCAASGSTFSSYAMSWVRQAPGKGLEW
VSAISGSGGSTYYADSVKGRFTISRDNSKNTLYLQMNSLRAEDTAVYYCA
TNSLYWYFDLWGRGTLVTVSSAS

In some embodiments, the anti-CTLA-4 scFv includes the amino acid sequence that is at least 90%, 91%, 92%, 93%, 94%, 95%, 96%, 97%, 98%, 99% or more identical to the amino acid sequence of SEQ ID NO: 117.

In some embodiments, the anti-CDR scFv includes the amino acid sequence:

Anti-CD3ϵ scFv
(SEQ ID NO: 118)
GGGSGGGGSGSGGGSGGGGSGGGQVQLQQSGAELARPGASVKMSCKASGY
TFTRYTMHWVKQRPGQGLEWIGYINPSRGYTNYNQKFKDKATLTTDKSSS
TAYMQLSSLTSEDSAVYYCARYYDDHYCLDYWGQGTTLTVSSGGGGSGGG
GSGGGGSQIVLTQSPAIMSASPGEKVTMTCSASSSVSYMNWYQQKSGTSP
KRWIYDTSKLASGVPAHFRGSGSGTSYSLTISGMEAEDAATYYCQQWSSN
PFTFGSGTKLEINR

In some embodiments, the anti-CD3ε scFv includes the amino acid sequence that is at least 90%, 91%, 92%, 93%, 94%, 95%, 96%, 97%, 98%, 99% or more identical to the amino acid sequence of SEQ ID NO: 118.

In some embodiments, the scFv is specific for binding one or more T-cells, one or more NK-cells and/or one or more macrophages. In some embodiments, the scFv is specific for binding a target selected from the group consisting of B7-H4, BTLA, CD3, CD4, CD8, CD16a, CD25, CD27, CD28, CD32, CD56, CD137, CTLA-4, GITR, HVEM, ICOS, LAG3, NKG2D, OX40, PD-1, TIGIT, TIM3, or VISTA.

In some embodiments, the multispecific AA also includes an agent conjugated to the AB. In some embodiments, the agent is a therapeutic agent. In some embodiments, the agent is an antineoplastic agent. In some embodiments, the agent is a toxin or fragment thereof. In some embodiments, the agent is conjugated to the multispecific AA via a linker. In some embodiments, the agent is conjugated to the AB via a cleavable linker. In some embodiments, the linker is a non-cleavable linker. In some embodiments, the agent is a microtubule inhibitor. In some embodiments, the agent is a nucleic acid damaging agent, such as a DNA alkylator or DNA intercalator, or other DNA damaging agent. In some embodiments, the linker is a cleavable linker. In some embodiments, the agent is an agent selected from the group listed in Table 1. In some embodiments, the agent is a dolastatin. In some embodiments, the agent is an auristatin or derivative thereof. In some embodiments, the agent is auristatin E or a derivative thereof. In some embodiments, the agent is monomethyl auristatin E (MMAE). In some embodiments, the agent is monomethyl auristatin D (MMAD). In some embodiments, the agent is a maytansinoid or maytansinoid derivative. In some embodiments, the agent is DM1 or DM4. In some embodiments, the agent is a duocarmycin or derivative thereof. In some embodiments, the agent is a calicheamicin or derivative thereof. In some embodiments, the agent is a pyrrolobenzodiazepine. In some embodiments, the agent is a pyrrolobenzodiazepine dimer.

In some embodiments, the multispecific AA also includes a detectable moiety. In some embodiments, the detectable moiety is a diagnostic agent.

In some embodiments, the multispecific AA naturally contains one or more disulfide bonds. In some embodiments, the multispecific AA can be engineered to include one or more disulfide bonds.

The disclosure also provides an isolated nucleic acid molecule encoding a multispecific AA described herein, as well as vectors that include these isolated nucleic acid sequences. The disclosure provides methods of producing a multispecific AA by culturing a cell under conditions that lead to expression of the activatable antibody, wherein the cell comprises such a nucleic acid molecule. In some embodiments, the cell comprises such a vector.

The disclosure also provides a method of manufacturing multispecific AAs of the disclosure by (a) culturing a cell comprising a nucleic acid construct that encodes the multispecific AA under conditions that lead to expression of the multispecific activatable, and (b) recovering the multispecific activatable antibody. Suitable AB, MM, and/or CM include any of the AB, MM, and/or CM disclosed herein.

The disclosure also provides multispecific AAs and/or multispecific AA compositions that include at least a first antibody or antigen-binding fragment thereof (AB1) that specifically binds a first target or first epitope and a second antibody or antigen-biding fragment thereof (AB2) that binds a second target or a second epitope, where at least AB1 is coupled or otherwise attached to a masking moiety (MM1), such that coupling of the MM1 reduces the ability of AB1 to bind its target. In some embodiments, the MM1 is coupled to AB1 via a first cleavable moiety (CM1) sequence that includes a substrate for a protease, for example, a protease that is co-localized with the target of AB1 at a treatment site or a diagnostic site in a subject. The multispecific AAs provided herein are stable in circulation, activated at intended sites of therapy and/or diagnosis but not in normal, i.e., healthy tissue, and, when activated, exhibit binding to the target of AB1 that is at least comparable to the corresponding, unmodified multispecific antibody. Suitable AB, MM, and/or CM include any of the AB, MM, and/or CM disclosed herein.

The disclosure also provides compositions and methods that include a multispecific AA that includes at least a first antibody or antibody fragment (AB1) that specifically binds a target and a second antibody or antibody fragment (AB2), where at least the first AB in the multispecific AA is coupled to a masking moiety (MM1) that decreases the ability of AB1 to bind its target. In some embodiments, each AB is coupled to a MM that decreases the ability of its corresponding AB to each target. For example, in bispecific AA embodiments, AB1 is coupled to a first masking moiety (MM1) that decreases the ability of AB1 to bind its target, and AB2 is coupled to a second masking moiety (MM2) that decreases the ability of AB2 to bind its target. In some embodiments, the multispecific AA comprises more than two AB regions; in such embodiments, AB1 is coupled to a first masking moiety (MM1) that decreases the ability of AB1 to bind its target, AB2 is coupled to a second masking moiety (MM2) that decreases the ability of AB2 to bind its target, AB3 is coupled to a third masking moiety (MM3) that decreases the ability of AB3 to bind its target, and so on for each AB in the multispecific activatable antibody. Suitable AB, MM, and/or CM include any of the AB, MM, and/or CM disclosed herein.

In some embodiments, the multispecific AA further includes at least one cleavable moiety (CM) that is a substrate for a protease, where the CM links a MM to an AB. For example, in some embodiments, the multispecific AA includes at least a first antibody or antibody fragment (AB1) that specifically binds a target and a second antibody or antibody fragment (AB2), where at least the first AB in the multispecific AA is coupled via a first cleavable moiety (CM1) to a masking moiety (MM1) that decreases the ability of AB1 to bind its target. In some bispecific AA embodiments, AB1 is coupled via CM1 to MM1, and AB2 is coupled via a second cleavable moiety (CM2) to a second masking moiety (MM2) that decreases the ability of AB2 to bind its target. In some embodiments, the multispecific AA comprises more than two AB regions; in some of these embodiments, AB1 is coupled via CM1 to MM1, AB2 is coupled via CM2 to MM2, and AB3 is coupled via a third cleavable moiety (CM3) to a third masking moiety (MM3) that decreases the ability of AB3 to bind its target, and so on for each AB in the multispecific activatable antibody. Suitable AB, MM, and/or CM include any of the AB, MM, and/or CM disclosed herein.

### Activatable Antibodies Having Non-Binding Steric Moieties or Binding Partners for Non-Binding Steric Moieties

The disclosure also provides AAs that include non-binding steric moieties (NB) or binding partners (BP) for non-binding steric moieties, where the BP recruits or otherwise attracts the NB to the activatable antibody. The AAs provided herein include, for example, an AA that includes a non-binding steric moiety (NB), a cleavable linker (CL) and antibody or antibody fragment (AB) that binds a target; an AA that includes a binding partner for a non-binding steric moiety (BP), a CL and an AB; and an AA that includes a BP to which an NB has been recruited, a CL and an AB that binds the target. AAs in which the NB is covalently linked to the CL and AB of the AA or is associated by interaction with a BP that is covalently linked to the CL and AB of the AA are referred to herein as “NB-containing activatable antibodies.” By activatable or switchable is meant that the AA exhibits a first level of binding to a target when the AA is in an inhibited, masked or uncleaved state (i.e., a first conformation), and a second level of binding to the target when the AA is in an uninhibited, unmasked and/or cleaved state (i.e., a second conformation, i.e., activated antibody), where the second level of target binding is greater than the first level of target binding. The AA compositions can exhibit increased bioavailability and more favorable biodistribution compared to conventional antibody therapeutics.

In some embodiments, AAs provide for reduced toxicity and/or adverse side effects that could otherwise result from binding of the at non-treatment sites and/or non-diagnostic sites if the AB were not masked or otherwise inhibited from binding to such a site.

Anti-CD166 AAs that include a non-binding steric moiety (NB) can be made using the methods set forth in PCT Publication No. WO 2013/192546, the contents of which are hereby incorporated by reference in their entirety.

### Production of Activatable Antibodies

The disclosure also provides methods of producing an activatable anti-CD166 antibody polypeptide by culturing a cell under conditions that lead to expression of the polypeptide, wherein the cell comprises an isolated nucleic acid molecule encoding an antibody and/or an AA described herein, and/or vectors that include these isolated nucleic acid sequences. The disclosure provides methods of producing an antibody and/or AA by culturing a cell under conditions that lead to expression of the antibody and/or activatable antibody, wherein the cell comprises an isolated nucleic acid molecule encoding an antibody and/or an AA described herein, and/or vectors that include these isolated nucleic acid sequences.

The invention also provides a method of manufacturing AAs that in an activated state binds CD166 by (a) culturing a cell comprising a nucleic acid construct that encodes the AA under conditions that lead to expression of the activatable antibody, wherein the AA comprises a masking moiety (MM), a cleavable moiety (CM), and an antibody or an antigen binding fragment thereof (AB) that specifically binds CD166, (i) wherein the CM is a polypeptide that functions as a substrate for a protease; and (ii) wherein the CM is positioned in the AA such that, when the AA is in an uncleaved state, the MM interferes with specific binding of the AB to CD166 and in a cleaved state the MM does not interfere or compete with specific binding of the AB to CD166; and (b) recovering the activatable antibody. Suitable AB, MM, and/or CM include any of the AB, MM, and/or CM disclosed herein.

The following exemplary nucleotide sequences are provided for use to make and use the AAs and conjugated AAs provided herein. Also provided are nucleotide sequences that are at least 90%, 95%, or even 99% homologous to the nucleotide sequences provided below.

Encoding amino acid sequence of SEQ ID NO: 239
Human αCD166 Heavy Chain (HuCD166_HcC) - Nucleotide sequence
SEQ ID NO: 241
CAGATCACCCTGAAAGAGTCCGGCCCCACCCTGGTGAAACCCACCCAGACCCTGACCC
TGACATGCACCTTCTCCGGCTTCAGCCTGTCCACCTACGGCATGGGCGTGGGCTGGATC
AGGCAGCCTCCTGGCAAGGCCCTGGAATGGCTGGCCAACATCTGGTGGTCCGAGGACA
AGCACTACTCCCCCAGCCTGAAGTCCCGGCTGACCATCACCAAGGACACCTCCAAGAA
CCAGGTGGTGCTGACAATCACAAACGTGGACCCCGTGGACACCGCCACCTACTACTGC
GTGCAGATCGACTACGGCAACGACTACGCCTTCACCTACTGGGGCCAGGGCACACTGG
TGACAGTGTCCTCCGCCTCCACCAAGGGCCCCTCCGTGTTCCCTCTGGCCCCTTCCAGC
AAGTCCACCTCTGGCGGCACAGCTGCCCTGGGCTGCCTGGTGAAAGACTACTTCCCCGA
GCCCGTGACCGTGTCCTGGAACTCTGGCGCCCTGACCAGCGGAGTGCACACCTTCCCTG
CCGTGCTGCAGTCCTCCGGCCTGTACTCCCTGTCCTCCGTGGTGACCGTGCCCTCCAGCT
CTCTGGGCACCCAGACCTACATCTGCAACGTGAACCACAAGCCCTCCAACACCAAGGT
GGACAAGAAGGTGGAACCCAAGTCCTGCGACAAGACCCACACCTGTCCCCCCTGCCCT
GCCCCTGAACTGCTGGGCGGACCTTCCGTGTTTCTGTTCCCCCCAAAGCCTAAGGACAC
CCTGATGATCTCCCGGACCCCCGAAGTGACCTGCGTGGTGGTGGACGTGTCCCACGAG
GACCCTGAAGTGAAGTTCAATTGGTACGTGGACGGCGTGGAAGTGCACAACGCCAAGA
CCAAGCCCAGAGAGGAACAGTACAACTCCACCTACCGGGTGGTGTCTGTGCTGACCGT
GCTGCACCAGGACTGGCTGAACGGCAAAGAGTACAAGTGCAAGGTGTCCAACAAGGC
CCTGCCTGCCCCCATCGAAAAGACCATCTCCAAGGCCAAGGGCCAGCCCCGCGAGCCT
CAGGTGTACACACTGCCCCCTAGCCGGGAAGAGATGACCAAGAATCAGGTGTCCCTGA
CCTGTCTGGTGAAAGGCTTCTACCCCTCCGATATCGCCGTGGAATGGGAGTCCAACGGC
CAGCCCGAGAACAACTACAAGACCACCCCCCCTGTGCTGGACTCCGACGGCTCATTCTT
CCTGTACTCCAAGCTGACCGTGGACAAGTCCCGGTGGCAGCAGGGCAACGTGTTCTCCT
GCAGCGTGATGCACGAGGCCCTGCACAACCACTACACCCAGAAGTCCCTGTCCCTGAG
CCCCGGCAAG
Encoding amino acid sequence of SEQ ID NO: 480
Human αCD166 Heavy Chain (HuCD166_HcC)-Des-HC Nucleotide sequence
SEQ ID NO: 481
CAGATCACCCTGAAAGAGTCCGGCCCCACCCTGGTGAAACCCACCCAGACCCTGACCC
TGACATGCACCTTCTCCGGCTTCAGCCTGTCCACCTACGGCATGGGCGTGGGCTGGATC
AGGCAGCCTCCTGGCAAGGCCCTGGAATGGCTGGCCAACATCTGGTGGTCCGAGGACA
AGCACTACTCCCCCAGCCTGAAGTCCCGGCTGACCATCACCAAGGACACCTCCAAGAA
CCAGGTGGTGCTGACAATCACAAACGTGGACCCCGTGGACACCGCCACCTACTACTGC
GTGCAGATCGACTACGGCAACGACTACGCCTTCACCTACTGGGGCCAGGGCACACTGG
TGACAGTGTCCTCCGCCTCCACCAAGGGCCCCTCCGTGTTCCCTCTGGCCCCTTCCAGC
AAGTCCACCTCTGGCGGCACAGCTGCCCTGGGCTGCCTGGTGAAAGACTACTTCCCCGA
GCCCGTGACCGTGTCCTGGAACTCTGGCGCCCTGACCAGCGGAGTGCACACCTTCCCTG
CCGTGCTGCAGTCCTCCGGCCTGTACTCCCTGTCCTCCGTGGTGACCGTGCCCTCCAGCT
CTCTGGGCACCCAGACCTACATCTGCAACGTGAACCACAAGCCCTCCAACACCAAGGT
GGACAAGAAGGTGGAACCCAAGTCCTGCGACAAGACCCACACCTGTCCCCCCTGCCCT
GCCCCTGAACTGCTGGGCGGACCTTCCGTGTTTCTGTTCCCCCCAAAGCCTAAGGACAC
CCTGATGATCTCCCGGACCCCCGAAGTGACCTGCGTGGTGGTGGACGTGTCCCACGAG
GACCCTGAAGTGAAGTTCAATTGGTACGTGGACGGCGTGGAAGTGCACAACGCCAAGA
CCAAGCCCAGAGAGGAACAGTACAACTCCACCTACCGGGTGGTGTCTGTGCTGACCGT
GCTGCACCAGGACTGGCTGAACGGCAAAGAGTACAAGTGCAAGGTGTCCAACAAGGC
CCTGCCTGCCCCCATCGAAAAGACCATCTCCAAGGCCAAGGGCCAGCCCCGCGAGCCT
CAGGTGTACACACTGCCCCCTAGCCGGGAAGAGATGACCAAGAATCAGGTGTCCCTGA
CCTGTCTGGTGAAAGGCTTCTACCCCTCCGATATCGCCGTGGAATGGGAGTCCAACGGC
CAGCCCGAGAACAACTACAAGACCACCCCCCCTGTGCTGGACTCCGACGGCTCATTCTT
CCTGTACTCCAAGCTGACCGTGGACAAGTCCCGGTGGCAGCAGGGCAACGTGTTCTCCT
GCAGCGTGATGCACGAGGCCCTGCACAACCACTACACCCAGAAGTCCCTGTCCCTGAG
CCCCGGC
Encoding amino acid sequence of SEQ ID NO: 246
Human αCD166 Light Chain (spacer-MM-LP1-CM-LP2-Ab)
[spacer (SEQ ID NO: 319)] [huCD166Lcl_7614.6_3001 (SEQ ID NO: 315)]
SEQ ID NO: 247
[CAGGGACAGTCTGGCCAGGGC][CTGTGTCACCCTGCTGTGCTGTCTGCCTGGGAGTCC
TGTTCCAGCGGCGGAGGCTCCTCTGGCGGCTCTGCTGTGGGCCTGCTGGCTCCACCTGG
CGGCCTGTCCGGCAGATCTGACAACCACGGCGGCTCCGACATCGTGATGACCCAGTCC
CCCCTGTCCCTGCCCGTGACTCCTGGCGAGCCTGCCTCCATCTCCTGCCGGTCCTCCAA
GTCCCTGCTGCACTCCAACGGCATCACCTACCTGTACTGGTATCTGCAGAAGCCCGGCC
AGTCCCCTCAGCTGCTGATCTACCAGATGTCCAACCTGGCCTCCGGCGTGCCCGACAGA
TTCTCCGGCTCTGGCTCCGGCACCGACTTCACCCTGAAGATCTCCCGGGTGGAAGCCGA
GGACGTGGGCGTGTACTACTGCGCCCAGAACCTGGAACTGCCCTACACCTTCGGCCAG
GGCACCAAGCTGGAAATCAAGCGGACCGTGGCCGCTCCCTCCGTGTTCATCTTCCCACC
CTCCGACGAGCAGCTGAAGTCCGGCACCGCCTCCGTGGTCTGCCTGCTGAACAACTTCT
ACCCCCGCGAGGCCAAGGTGCAGTGGAAGGTGGACAACGCCCTGCAGTCCGGCAACTC
CCAGGAATCCGTCACCGAGCAGGACTCCAAGGACAGCACCTACTCCCTGTCCTCCACC
CTGACCCTGTCCAAGGCCGACTACGAGAAGCACAAGGTGTACGCCTGCGAAGTGACCC
ACCAGGGACTGAGCAGCCCCGTGACCAAGTCCTTCAACCGGGGCGAGTGC]
Encoding amino acid sequence of SEQ ID NO: 314
Human aCD166 Light Chain (MM-LP1-CM-LP2-Ab)
huCD166Lcl_7614.6_3001
SEQ ID NO: 315
CTGTGTCACCCTGCTGTGCTGTCTGCCTGGGAGTCCTGTTCCAGCGGCGGAGGCTCCTC
TGGCGGCTCTGCTGTGGGCCTGCTGGCTCCACCTGGCGGCCTGTCCGGCAGATCTGACA
ACCACGGCGGCTCCGACATCGTGATGACCCAGTCCCCCCTGTCCCTGCCCGTGACTCCT
GGCGAGCCTGCCTCCATCTCCTGCCGGTCCTCCAAGTCCCTGCTGCACTCCAACGGCAT
CACCTACCTGTACTGGTATCTGCAGAAGCCCGGCCAGTCCCCTCAGCTGCTGATCTACC
AGATGTCCAACCTGGCCTCCGGCGTGCCCGACAGATTCTCCGGCTCTGGCTCCGGCACC
GACTTCACCCTGAAGATCTCCCGGGTGGAAGCCGAGGACGTGGGCGTGTACTACTGCG
CCCAGAACCTGGAACTGCCCTACACCTTCGGCCAGGGCACCAAGCTGGAAATCAAGCG
GACCGTGGCCGCTCCCTCCGTGTTCATCTTCCCACCCTCCGACGAGCAGCTGAAGTCCG
GCACCGCCTCCGTGGTCTGCCTGCTGAACAACTTCTACCCCCGCGAGGCCAAGGTGCAG
TGGAAGGTGGACAACGCCCTGCAGTCCGGCAACTCCCAGGAATCCGTCACCGAGCAGG
ACTCCAAGGACAGCACCTACTCCCTGTCCTCCACCCTGACCCTGTCCAAGGCCGACTAC
GAGAAGCACAAGGTGTACGCCTGCGAAGTGACCCACCAGGGACTGAGCAGCCCCGTG
ACCAAGTCCTTCAACCGGGGCGAGTGC
Nucleotide Sequence Encoding SEQ ID NO: 305 Spacer
SEQ ID NO: 319
CAGGGACAGTCTGGCCAGGGC

### Therapeutic Use of Activatable Antibodies, and Conjugated Activatable Antibodies

The disclosure provides methods of treating, preventing and/or delaying the onset or progression of, or alleviating a symptom associated with aberrant expression and/or activity of CD166 in a subject using AAs that bind CD166, particularly AAs that bind and neutralize or otherwise inhibit at least one biological activity of CD166 and/or CD166-mediated signaling.

The disclosure also provides methods of treating, preventing and/or delaying the onset or progression of, or alleviating a symptom associated with the presence, growth, proliferation, metastasis, and/or activity of cells which are expressing CD166 or aberrantly expressing CD166 in a subject using AAs that bind CD166, particularly AAs that bind, target, neutralize, kill, or otherwise inhibit at least one biological activity of cells which are expressing or aberrantly expressing CD166.

The disclosure also provides methods of treating, preventing and/or delaying the onset or progression of, or alleviating a symptom associated with the presence, growth, proliferation, metastasis, and/or activity of cells which are expressing CD166 in a subject using AAs that bind CD166, particularly AAs that bind, target, neutralize, kill, or otherwise inhibit at least one biological activity of cells which are expressing CD166.

The disclosure also provides methods of treating, preventing and/or delaying the onset or progression of, or alleviating a symptom associated with the presence, growth, proliferation, metastasis, and/or activity of cells which are aberrantly expressing CD166 in a subject using AAs that bind CD166, particularly AAs that bind, target, neutralize, kill, or otherwise inhibit at least one biological activity of cells which are aberrantly expressing CD166.

The disclosure also provides methods of preventing, delaying the progression of, treating, alleviating a symptom of, or otherwise ameliorating cancer in a subject by administering a therapeutically effective amount of an anti-CD166 antibody, conjugated anti-CD166 antibody, activatable anti-CD166 antibody and/or conjugated activatable anti-CD166 antibody described herein to a subject in need thereof.

The disclosure also provides AAs that bind CD166, particularly AAs that bind and neutralize or otherwise inhibit at least one biological activity of CD166 and/or CD166 signaling, for use in treating, preventing and/or delaying the onset or progression of, or alleviating a symptom associated with aberrant expression and/or activity of CD166 in a subject.

The disclosure also provides AAs that bind CD166, particularly AAs that bind, target, neutralize, kill, or otherwise inhibit at least one biological activity of cells which are expressing or aberrantly expressing CD166, for use in treating, preventing and/or delaying the onset or progression of, or alleviating a symptom associated with the presence, growth, proliferation, metastasis, and/or activity of cells which are expressing or aberrantly expressing CD166 in a subject.

The disclosure also provides an anti-CD166 antibody, conjugated anti-CD166 antibody, activatable anti-CD166 antibody and/or conjugated activatable anti-CD166 antibody described herein, for use in preventing, delaying the progression of, treating, alleviating a symptom of, or otherwise ameliorating cancer in a subject, wherein the antibody is for administration in a therapeutically effective amount.

By way of non-limiting example, the AAs of the disclosure can be used for treating, preventing and/or delaying the onset or progression of an epithelial or squamous cell cancer, a carcinoid, and/or a neuroendocrine cancer. Examples of cancers include, but are not limited to, adenocarcinoma, bile duct (biliary) cancer, bladder cancer, breast cancer, e.g., triple-negative breast cancer, Her2-negative breast cancer, estrogen receptor-positive breast cancer; carcinoid cancer; cervical cancer; cholangiocarcinoma; colorectal; endometrial; glioma; head and neck cancer, e.g., head and neck squamous cell cancer; leukemia; liver cancer; lung cancer, e.g., NSCLC, SCLC; lymphoma; melanoma; osopharyngeal cancer; ovarian cancer; pancreatic cancer; prostate cancer, e.g., metastatic castration-resistant prostate carcinoma; renal cancer; skin cancer; squamous cell cancer; stomach cancer; testis cancer; thyroid cancer; and urothelial cancer.

In some embodiments, the cancer is any epithelial or squamous cancer. In some embodiments, the cancer is prostate cancer, breast cancer, lung cancer, cervical cancer, oropharyngeal cancer, and/or head and neck cancer.

In some embodiments, the cancer is a bladder cancer, a bone cancer, a breast cancer, a carcinoid, a cervical cancer, a colorectal cancer, a colon cancer, an endometrial cancer, an epithelial cancer, a glioma, a head and neck cancer, a liver cancer, a lung cancer, a melanoma, an oropharyngeal cancer, an ovarian cancer, a pancreatic cancer, a prostate cancer, a renal cancer, a sarcoma, a skin cancer, a stomach cancer, a testis cancer, a thyroid cancer, a urogenital cancer, and/or a urothelial cancer.

In some embodiments, the cancer is selected from the group consisting of triple negative breast cancer (TNBC), non-small cell lung cancer (NSCLC), small cell lung cancer (SCLC), Ras mutant colorectal carcinoma, a rare epithelial cancer, oropharyngeal cancer, cervical cancer, head and neck squamous cell carcinoma (HNSCC), and/or prostate cancer. In some embodiments, the cancer is associated with a CD166-expressing tumor. In some embodiments, the cancer is due to a CD166-expressing tumor.

An anti-CD166 antibody, a conjugated anti-CD166 antibody, an activatable anti-CD166 antibody and/or a conjugated activatable anti-CD166 antibody used in any of the embodiments of these methods and uses can be administered at any stage of the disease. For example, such an anti-CD166 antibody, conjugated anti-CD166 antibody, activatable anti-CD166 antibody and/or conjugated activatable anti-CD166 antibody can be administered to a subject suffering cancer of any stage, from early to metastatic.

In exemplary embodiments the subject is suffering from, or suspected to be suffering from breast carcinoma, castration-resistant prostate cancer (CPRC), cholangiocarcinoma, endometrial carcinoma, epithelial ovarian carcinoma, head and neck squamous cell carcinoma (HNSCC), and non-small cell lung cancer (NSCLC).

In exemplary embodiments the subject is suffering from, or suspected to be suffering from, a skin lesion. In some embodiments, the skin lesion is a skin metastasis.

As provided herein, the subject to be treated is a mammal, such as a human, non-human primate, companion animal (e.g., cat, dog, horse), farm animal, work animal, or zoo animal. In some embodiments, the subject is a human. In some embodiments, the subject is a companion animal. In some embodiments, the subject is an animal in the care of a veterinarian.

In some embodiments, a subject suffering from, or suspected to be suffering from a breast carcinoma, who receives an AA of the present disclosure, e.g. Combination 55 or Combination 60, has an estrogen receptor expressing (ER+) tumor and should have received anti-hormonal therapy and has experienced disease progression prior to being treated with the AA of the present disclosure. In some embodiments, a subject suffering from, or suspected to be suffering from a breast carcinoma, who receives an AA of the present disclosure, e.g. Combination 55 or Combination 60, has a triple negative breast carcinoma (TNBC) and has received ≥2 prior lines of therapy prior to being treated with the AA of the present disclosure.

In some embodiments, a subject suffering from, or suspected to be suffering from a castration-resistant prostate carcinoma, who receives an AA of the present disclosure, e.g. Combination 55 or Combination 60, has received ≥1 prior therapy, before being treated with the AA of the present disclosure.

In some embodiments, a subject suffering from, or suspected to be suffering from a cholangiocarcinoma, who receives an AA of the present disclosure, e.g. Combination 55 or Combination 60, has failed ≥1 prior line of gemcitabine-containing regimen, before being treated with the AA of the present disclosure.

In some embodiments, a subject suffering from, or suspected to be suffering from a endometrial carcinoma, who receives an AA of the present disclosure, e.g. Combination 55 or Combination 60, has received ≥1 platinum-containing regimen for extra-uterine or advanced disease, before being treated with the AA of the present disclosure.

In some embodiments, a subject suffering from, or suspected to be suffering from a epithelial ovarian carcinoma, who receives an AA of the present disclosure, e.g. Combination 55 or Combination 60, either has a non-breast cancer (BRCA) mutation (germline or somatic), or has an unknown BRCA mutational status and has platinum-resistant or platinum refractory ovarian carcinoma. In some embodiments, a subject suffering from, or suspected to be suffering from an epithelial ovarian carcinoma, who receives an AA of the present disclosure, e.g. Combination 55 or Combination 60, has a BRCA mutation and is refractory to, or otherwise ineligible for, PARP inhibitors.

In some embodiments, a subject suffering from, or suspected to be suffering from a HNSCC, who receives an AA of the present disclosure, e.g. Combination 55 or Combination 60, has received ≥1 platinum-containing regimen and a PD-1/PD-L1 inhibitor (if approved for the subject's indication and locality), before being treated with the AA of the present disclosure.

In some embodiments, a subject suffering from, or suspected to be suffering from a NSCLC, who receives an AA of the present disclosure, e.g. Combination 55 or Combination 60, has received ≥1 platinum-containing regimen before being treated with the AA of the present disclosure. In some embodiments, a subject suffering from, or suspected to be suffering from a NSCLC, who receives an AA of the present disclosure, e.g. Combination 55 or Combination 60, has been previously administered a checkpoint inhibitor (if approved for the subject's indication in their locality) before being treated with the AA of the present disclosure.

In some embodiments, a subject who has any of the following may not be eligible to receive an AA of the present disclosure for the treatment of breast carcinoma, castration-resistant prostate cancer (CPRC), cholangiocarcinoma, endometrial carcinoma, epithelial ovarian carcinoma, HNSCC, and NSCLC: active or chronic corneal disorder, history of corneal transplantation, active herpetic keratitis, and active ocular conditions requiring ongoing treatment/monitoring; serious concurrent illness, including clinically relevant active infection; history of or current active autoimmune diseases; significant cardiac disease such as recent myocardial infarction; history of multiple sclerosis or other demyelinating disease, Eaton-Lambert syndrome (para-neoplastic syndrome), history of hemorrhagic or ischemic stroke within the last 6 months, or alcoholic liver disease; non-healing wound(s) or ulcer(s) except for ulcerative lesions caused by the underlying neoplasm; history of severe allergic or anaphylactic reactions to previous monoclonal antibody therapy; currently receiving anticoagulation therapy with warfarin; or major surgery (requiring general anesthesia) within 3 months prior to dosing.

Activatable anti-CD166 antibody and/or conjugated activatable anti-CD166 antibody and therapeutic formulations thereof are administered to a subject suffering from or susceptible to a disease or disorder associated with aberrant CD166 expression and/or activity. A subject suffering from or susceptible to a disease or disorder associated with aberrant CD166 expression and/or activity is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of an anti-CD166 antibody, conjugated anti-CD166 antibody, activatable anti-CD166 antibody and/or conjugated activatable anti-CD166 antibody to a subject suffering from a disease or disorder associated with aberrant CD166 expression and/or activity is considered successful if any of a variety of laboratory or clinical objectives is achieved. For example, administration of an anti-CD166 antibody, conjugated anti-CD166 antibody, activatable anti-CD166 antibody and/or conjugated activatable anti-CD166 antibody to a subject suffering from a disease or disorder associated with aberrant CD166 expression and/or activity is considered successful if one or more of the symptoms associated with the disease or disorder is alleviated, reduced, inhibited or does not progress to a further, i.e., worse, state. Administration of an anti-CD166 antibody, conjugated anti-CD166 antibody, activatable anti-CD166 antibody and/or conjugated activatable anti-CD166 antibody to a subject suffering from a disease or disorder associated with aberrant CD166 expression and/or activity is considered successful if the disease or disorder enters remission or does not progress to a further, i.e., worse, state.

In some embodiments, activatable anti-CD166 antibody and/or conjugated activatable anti-CD166 antibody and therapeutic formulations thereof are administered to a subject suffering from or susceptible to a disease or disorder, such as subjects suffering from cancer or other neoplastic condition, wherein the subject's diseased cells are expressing CD166. In some embodiments, the diseased cells are associated with aberrant CD166 expression and/or activity. In some embodiments, the diseased cells are associated with normal CD166 expression and/or activity. A subject suffering from or susceptible to a disease or disorder wherein the subject's diseased cells express CD166 is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

In some embodiments, activatable anti-CD166 antibody and/or conjugated activatable anti-CD166 antibody and therapeutic formulations thereof are administered to a subject suffering from or susceptible to a disease or disorder associated with cells expressing CD166 or the presence, growth, proliferation, metastasis, and/or activity of such cells, such as subjects suffering from cancer or other neoplastic conditions. In some embodiments, the cells are associated with aberrant CD166 expression and/or activity. In some embodiments, the cells are associated with normal CD166 expression and/or activity. A subject suffering from or susceptible to a disease or disorder associated with cells that express CD166 is identified using any of a variety of methods known in the art. For example, subjects suffering from cancer or other neoplastic condition are identified using any of a variety of clinical and/or laboratory tests such as, physical examination and blood, urine and/or stool analysis to evaluate health status. For example, subjects suffering from inflammation and/or an inflammatory disorder are identified using any of a variety of clinical and/or laboratory tests such as physical examination and/or bodily fluid analysis, e.g., blood, urine and/or stool analysis, to evaluate health status.

Administration of an anti-CD166 antibody, conjugated anti-CD166 antibody, activatable anti-CD166 antibody and/or conjugated activatable anti-CD166 antibody to a subject suffering from a disease or disorder associated with cells expressing CD166 is considered successful if any of a variety of laboratory or clinical objectives is achieved. For example, administration of an anti-CD166 antibody, conjugated anti-CD166 antibody, activatable anti-CD166 antibody and/or conjugated activatable anti-CD166 antibody to a subject suffering from a disease or disorder associated with cells expressing CD166 is considered successful if one or more of the symptoms associated with the disease or disorder is alleviated, reduced, inhibited or does not progress to a further, i.e., worse, state. Administration of an anti-CD166 antibody, conjugated anti-CD166 antibody, activatable anti-CD166 antibody and/or conjugated activatable anti-CD166 antibody to a subject suffering from a disease or disorder associated with cells expressing CD166 is considered successful if the disease or disorder enters remission or does not progress to a further, i.e., worse, state.

In some embodiments, activatable anti-CD166 antibody and/or conjugated activatable anti-CD166 antibody is administered during and/or after treatment in combination with one or more additional agents such as, for example, a chemotherapeutic agent, an anti-inflammatory agent, and/or an immunosuppressive agent. In some embodiments, activatable anti-CD166 antibody and/or conjugated activatable anti-CD166 antibody and the additional agent(s) are administered simultaneously. For example, activatable anti-CD166 antibody and/or conjugated activatable anti-CD166 antibody and the additional agent(s) can be formulated in a single composition or administered as two or more separate compositions. In some embodiments, activatable anti-CD166 antibody and/or conjugated activatable anti-CD166 antibody and the additional agent(s) are administered sequentially.

In some embodiments, activatable anti-CD166 antibodies and/or conjugated activatable anti-CD166 antibodies described herein are used in conjunction with one or more additional agents or a combination of additional agents. Suitable additional agents include current pharmaceutical and/or surgical therapies for an intended application, such as, for example, cancer. For example, the anti-CD166 antibodies, conjugated anti-CD166 antibodies, activatable anti-CD166 antibodies and/or conjugated activatable anti-CD166 antibodies can be used in conjunction with an additional chemotherapeutic or anti-neoplastic agent.

In some embodiments, the additional agent(s) is a chemotherapeutic agent, such as a chemotherapeutic agent selected from the group consisting of docetaxel, paclitaxel, abraxane (i.e., albumin-conjugated paclitaxel), doxorubicin, oxaliplatin, carboplatin, cisplatin, irinotecan, and gemcitabine.

In some embodiments, the additional agent(s) is a checkpoint inhibitor, a kinase inhibitor, an agent targeting inhibitors in the tumor microenvironment, and/or a T cell or NK agonist. In some embodiments, the additional agent(s) is radiation therapy, alone or in combination with another additional agent(s) such as a chemotherapeutic or anti-neoplastic agent. In some embodiments, the additional agent(s) is a vaccine, an oncovirus, and/or a DC-activating agent such as, by way of non-limiting example, a toll-like receptor (TLR) agonist and/or α-CD40. In some embodiments, the additional agent(s) is a tumor-targeted antibody designed to kill the tumor via ADCC or via direct conjugation to a toxin (e.g., an antibody drug conjugate (ADC).

In some embodiments, the checkpoint inhibitor is an inhibitor of a target selected from the group consisting of CTLA-4, LAG-3, PD-1, CD166, TIGIT, TIM-3, B7H4, and Vista. In some embodiments, the kinase inhibitor is selected from the group consisting of B-RAFi, MEKi, and Btk inhibitors, such as ibrutinib. In some embodiments, the kinase inhibitor is crizotinib. In some embodiments, the tumor microenvironment inhibitor is selected from the group consisting of an IDO inhibitor, an α-CSF1R inhibitor, an α-CCR4 inhibitor, a TGF-beta, a myeloid-derived suppressor cell, or a T-regulatory cell. In some embodiments, the agonist is selected from the group consisting of Ox40, GITR, CD137, ICOS, CD27, and HVEM.

In some embodiments, the inhibitor is a CTLA-4 inhibitor. In some embodiments, the inhibitor is a LAG-3 inhibitor. In some embodiments, the inhibitor is a PD-1 inhibitor. In some embodiments, the inhibitor is a CD166 inhibitor. In some embodiments, the inhibitor is a TIGIT inhibitor. In some embodiments, the inhibitor is a TIM-3 inhibitor. In some embodiments, the inhibitor is a B7H4 inhibitor. In some embodiments, the inhibitor is a Vista inhibitor. In some embodiments, the inhibitor is a B-RAFi inhibitor. In some embodiments, the inhibitor is a MEKi inhibitor. In some embodiments, the inhibitor is a Btk inhibitor. In some embodiments, the inhibitor is ibrutinib. In some embodiments, the inhibitor is crizotinib. In some embodiments, the inhibitor is an IDO inhibitor. In some embodiments, the inhibitor is an α-CSF1R inhibitor. In some embodiments, the inhibitor is an α-CCR4 inhibitor. In some embodiments, the inhibitor is a TGF-beta. In some embodiments, the inhibitor is a myeloid-derived suppressor cell. In some embodiments, the inhibitor is a T-regulatory cell.

In some embodiments, the agonist is Ox40. In some embodiments, the agonist is GITR. In some embodiments, the agonist is CD137. In some embodiments, the agonist is ICOS. In some embodiments, the agonist is CD27. In some embodiments, the agonist is HVEM.

In some embodiments, the AA and/or conjugated AA is administered during and/or after treatment in combination with one or more additional agents such as, for example, a chemotherapeutic agent, an anti-inflammatory agent, and/or an immunosuppressive agent. In some embodiments, activatable anti-CD166 antibody and/or conjugated activatable anti-CD166 antibody and the additional agent are formulated into a single therapeutic composition, and activatable anti-CD166 antibody and/or conjugated activatable anti-CD166 antibody and additional agent are administered simultaneously. Alternatively, activatable anti-CD166 antibody and/or conjugated activatable anti-CD166 antibody and additional agent are separate from each other, e.g., each is formulated into a separate therapeutic composition, and activatable anti-CD166 antibody and/or conjugated activatable anti-CD166 antibody and the additional agent are administered simultaneously, or activatable anti-CD166 antibody and/or conjugated activatable anti-CD166 antibody and the additional agent are administered at different times during a treatment regimen. For example, activatable anti-CD166 antibody and/or conjugated activatable anti-CD166 antibody is administered prior to the administration of the additional agent, activatable anti-CD166 antibody and/or conjugated activatable anti-CD166 antibody is administered subsequent to the administration of the additional agent, or activatable anti-CD166 antibody and/or conjugated activatable anti-CD166 antibody and the additional agent are administered in an alternating fashion. As described herein, activatable anti-CD166 antibody and/or conjugated activatable anti-CD166 antibody and additional agent are administered in single doses or in multiple doses.

In some embodiments, activatable anti-CD166 antibody and/or conjugated activatable anti-CD166 antibody and the additional agent(s) are administered simultaneously. For example, activatable anti-CD166 antibody and/or conjugated activatable anti-CD166 antibody and the additional agent(s) can be formulated in a single composition or administered as two or more separate compositions. In some embodiments, activatable anti-CD166 antibody and/or conjugated activatable anti-CD166 antibody and the additional agent(s) are administered sequentially, or activatable anti-CD166 antibody and/or conjugated activatable anti-CD166 antibody and the additional agent are administered at different times during a treatment regimen.

In some embodiments, activatable anti-CD166 antibody and/or conjugated activatable anti-CD166 antibody is administered during and/or after treatment in combination with one or more additional agents such as, by way of non-limiting example, a chemotherapeutic agent, an anti-inflammatory agent, and/or an immunosuppressive agent, such as an alkylating agent, an anti-metabolite, an anti-microtubule agent, a topoisomerase inhibitor, a cytotoxic antibiotic, and/or any other nucleic acid damaging agent. In some embodiments, the additional agent is a taxane, such as paclitaxel (e.g., Abraxane®). In some embodiments, the additional agent is an anti-metabolite, such as gemcitabine. In some embodiments, the additional agent is an alkylating agent, such as platinum-based chemotherapy, such as carboplatin or cisplatin. In some embodiments, the additional agent is a targeted agent, such as a kinase inhibitor, e.g., sorafenib or erlotinib. In some embodiments, the additional agent is a targeted agent, such as another antibody, e.g., a monoclonal antibody (e.g., bevacizumab), a bispecific antibody, or a multispecific antibody. In some embodiments, the additional agent is a proteosome inhibitor, such as bortezomib or carfilzomib. In some embodiments, the additional agent is an immune modulating agent, such as lenolidominde or IL-2. In some embodiments, the additional agent is radiation. In some embodiments, the additional agent is an agent considered standard of care by those skilled in the art. In some embodiments, the additional agent is a chemotherapeutic agent well known to those skilled in the art.

In some embodiments, the additional agent is another antibody or antigen-binding fragment thereof, another conjugated antibody or antigen-binding fragment thereof, another AA or antigen-binding fragment thereof and/or another conjugated AA or antigen-binding fragment thereof. In some embodiments the additional agent is another antibody or antigen-binding fragment thereof, another conjugated antibody or antigen-binding fragment thereof, another AA or antigen-binding fragment thereof and/or another conjugated AA or antigen-binding fragment thereof against the same target as the first antibody or antigen-binding fragment thereof, the first conjugated antibody or antigen-binding fragment thereof, AA or antigen-binding fragment thereof and/or a conjugated AA or antigen-binding fragment thereof, e.g., against CD166. In some embodiments the additional agent is another antibody or antigen-binding fragment thereof, another conjugated antibody or antigen-binding fragment thereof, another AA or antigen-binding fragment thereof and/or another conjugated AA or antigen-binding fragment thereof against a target different than the target of the first antibody or antigen-binding fragment thereof, the first conjugated antibody or antigen-binding fragment thereof, AA or antigen-binding fragment thereof and/or a conjugated AA or antigen-binding fragment thereof.

In some embodiments, the additional antibody or antigen binding fragment thereof, conjugated antibody or antigen binding fragment thereof, AA or antigen binding fragment thereof, and/or conjugated AA or antigen binding fragment thereof is a monoclonal antibody, domain antibody, single chain, Fab fragment, a F(ab′)₂ fragment, a scFv, a scAb, a dAb, a single domain heavy chain antibody, or a single domain light chain antibody. In some embodiments, the additional antibody or antigen binding fragment thereof, conjugated antibody or antigen binding fragment thereof, AA or antigen binding fragment thereof, and/or conjugated AA or antigen binding fragment thereof is a mouse, other rodent, chimeric, humanized or fully human monoclonal antibody.

It will be appreciated that administration of therapeutic entities in accordance with the disclosure will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present disclosure, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

Therapeutic formulations of the disclosure, which include an activatable anti-CD166 antibody, such as by way of non-limiting example, AA and/or a conjugated AA, are used to prevent, treat or otherwise ameliorate a disease or disorder associated with aberrant target expression and/or activity. For example, therapeutic formulations of the disclosure, which include an AA and/or a conjugated activatable antibody, are used to treat or otherwise ameliorate a cancer or other neoplastic condition, inflammation, an inflammatory disorder, and/or an autoimmune disease. In some embodiments, the cancer is a solid tumor or a hematologic malignancy where the target is expressed. In some embodiments, the cancer is a solid tumor where the target is expressed. In some embodiments, the cancer is a hematologic malignancy where the target is expressed. In some embodiments, the target is expressed on parenchyma (e.g., in cancer, the portion of an organ or tissue that often carries out function(s) of the organ or tissue). In some embodiments, the target is expressed on a cell, tissue, or organ. In some embodiments, the target is expressed on stroma (i.e., the connective supportive framework of a cell, tissue, or organ). In some embodiments, the target is expressed on an osteoblast. In some embodiments, the target is expressed on the endothelium (vasculature). In some embodiments, the target is expressed on a cancer stem cell. In some embodiments, the agent to which the AA is conjugated is a microtubule inhibitor. In some embodiments, the agent to which the AA is conjugated is a nucleic acid damaging agent.

Efficaciousness of prevention, amelioration or treatment is determined in association with any known method for diagnosing or treating the disease or disorder associated with target expression and/or activity, such as, for example, aberrant target expression and/or activity. Prolonging the survival of a subject or otherwise delaying the progression of the disease or disorder associated with target expression and/or activity, e.g., aberrant target expression and/or activity, in a subject indicates that the AA and/or conjugated AA confers a clinical benefit.

An AA and/or a conjugated AA can be administered in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

In some embodiments where antibody fragments are used, the smallest fragment that specifically binds to the binding domain of the target protein is selected. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al., Proc. Natl. Acad. Sci. USA, 90: 7889-7893 (1993)). The formulation can also contain more than one active compound as necessary for the particular indication being treated, for example, in some embodiments, those with complementary activities that do not adversely affect each other. In some embodiments, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

### Diagnostic Uses

The invention also provides methods and kits for using the activatable anti-CD166 antibodies and/or conjugated activatable anti-CD166 antibodies in a variety of diagnostic and/or prophylactic indications. For example, the invention provides methods and kits for detecting the presence or absence of a cleaving agent and a target of interest in a subject or a sample by (i) contacting a subject or sample with an anti-CD166 activatable antibody, wherein the anti-CD166 AA comprises a masking moiety (MM), a cleavable moiety (CM) that is cleaved by the cleaving agent, and an antigen binding domain or fragment thereof (AB) that specifically binds the target of interest, wherein the anti-CD166 AA in an uncleaved, non-activated state comprises a structural arrangement from N-terminus to C-terminus as follows: MM-CM-AB or AB-CM-MM; (a) wherein the MM is a peptide that inhibits binding of the AB to CD166, and wherein the MM does not have an amino acid sequence of a naturally occurring binding partner of the AB and is not a modified form of a natural binding partner of the AB; and (b) wherein, when the AB is in an uncleaved, non-activated state, the MM interferes with specific binding of the AB to CD166, and when the AB is in a cleaved, activated state the MM does not interfere or compete with specific binding of the AB to CD166; and (ii) measuring a level of activated anti-CD166 AA in the subject or sample, wherein a detectable level of activated anti-CD166 AA in the subject or sample indicates that the cleaving agent and CD166 are present in the subject or sample and wherein no detectable level of activated anti-CD166 AA in the subject or sample indicates that the cleaving agent, CD166 or both the cleaving agent and CD166 are absent in the subject or sample.

In some embodiments, the activatable anti-CD166 antibody is an activatable anti-CD166 antibody to which a therapeutic agent is conjugated. In some embodiments, the activatable anti-CD166 antibody is not conjugated to an agent. In some embodiments, the activatable anti-CD166 antibody comprises a detectable label. In some embodiments, the detectable label is positioned on the AB. In some embodiments, measuring the level of activatable anti-CD166 antibody in the subject or sample is accomplished using a secondary reagent that specifically binds to the activated antibody, wherein the reagent comprises a detectable label. In some embodiments, the secondary reagent is an antibody comprising a detectable label.

In some embodiments of these methods and kits, the activatable anti-CD166 antibody includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or β-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods and kits, the subject is a mammal. In some embodiments of these methods, the subject is a human. In some embodiments, the subject is a non-human mammal, such as a non-human primate, companion animal (e.g., cat, dog, horse), farm animal, work animal, or zoo animal. In some embodiments, the subject is a rodent.

In some embodiments of these methods and kits, the method is an in vivo method. In some embodiments of these methods, the method is an in situ method. In some embodiments of these methods, the method is an ex vivo method. In some embodiments of these methods, the method is an in vitro method.

In some embodiments of the methods and kits, the method is used to identify or otherwise refine a patient population suitable for treatment with an anti-CD166 AA of the disclosure, followed by treatment by administering that activatable anti-CD166 antibody and/or conjugated activatable anti-CD166 antibody to a subject in need thereof. For example, patients that test positive for both the target (e.g., CD166) and a protease that cleaves the substrate in the CM (CM) of the anti-CD166 AA being tested in these methods are identified as suitable candidates for treatment with such an anti-CD166 AA comprising such a CM, and the patient is then administered a therapeutically effective amount of the activatable anti-CD166 antibody and/or conjugated activatable anti-CD166 antibody that was tested. Likewise, patients that test negative for either or both of the target (e.g., CD166) and the protease that cleaves the substrate in the CM in the AA being tested using these methods might be identified as suitable candidates for another form of therapy. In some embodiments, such patients can be tested with other anti-CD166 AAs until a suitable anti-CD166 AA for treatment is identified (e.g., an anti-CD166 AA comprising a CM that is cleaved by the patient at the site of disease). In some embodiments, the patient is then administered a therapeutically effective amount of the activatable anti-CD166 antibody and/or conjugated for which the patient tested positive. Suitable AB, MM, and/or CM include any of the AB, MM, and/or CM disclosed herein.

In some embodiments, the AA and/or conjugated AA contains a detectable label. An intact antibody, or a fragment thereof (e.g., Fab, scFv, or F(ab)₂) is used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the disclosure can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, immunochemical staining, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Accordingly, the AAs and conjugated AAs of the disclosure are also useful in a variety of diagnostic and prophylactic formulations. In one embodiment, an AA and/or a conjugated AA is administered to subjects that are at risk of developing one or more of the aforementioned disorders. A subject's or organ's predisposition to one or more of the aforementioned disorders can be determined using genotypic, serological or biochemical markers.

In some embodiments of the disclosure, an AA and/or a conjugated AA is administered to human individuals diagnosed with a clinical indication associated with one or more of the aforementioned disorders. Upon diagnosis, an AA and/or a conjugated AA is administered to mitigate or reverse the effects of the clinical indication.

An activatable antibody, and/or a conjugated AA of the disclosure is also useful in the detection of a target in subject samples and accordingly are useful as diagnostics. For example, the antibodies and/or activatable antibodies, and conjugated versions thereof, of the disclosure are used in in vitro assays, e.g., ELISA, to detect target levels in a subject sample.

In one embodiment, an AA and/or a conjugated AA of the disclosure is immobilized on a solid support (e.g., the well(s) of a microtiter plate). The immobilized AA and/or conjugated AA serves as a capture antibody for any target that may be present in a test sample. Prior to contacting the immobilized activatable antibody, and/or conjugated versions thereof, with a subject sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen in the test sample is determined by comparison with a standard curve developed from the standard samples.

It will be appreciated that based on the results obtained using the AAs of the disclosure, and conjugated versions thereof, in an in vitro diagnostic assay, it is possible to stage a disease in a subject based on expression levels of the target antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

An AA and/or a conjugated AA can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, AAs having an enzymatically cleavable CM can be used to detect the presence or absence of an enzyme that is capable of cleaving the CM. Such AAs can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable antibody) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM can be selected to be substrate for at least one protease found at the site of a tumor, at the site of a viral or bacterial infection at a biologically confined site (e.g., such as in an abscess, in an organ, and the like), and the like. The AB can be one that binds a target antigen. Using methods as disclosed herein, or when appropriate, methods familiar to one skilled in the art, a detectable label (e.g., a fluorescent label or radioactive label or radiotracer) can be conjugated to an AB or other region of an antibody and/or activatable antibody. Suitable detectable labels are discussed in the context of the above screening methods and additional specific examples are provided below. Using an AB specific to a protein or peptide of the disease state, along with at least one protease whose activity is elevated in the disease tissue of interest, AAs will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, AAs can be used to detect the presence or absence of a cleaving agent in a sample. For example, where the AAs contain a CM susceptible to cleavage by an enzyme, the AAs can be used to detect (either qualitatively or quantitatively) the presence of an enzyme in the sample. In another example, where the AAs contain a CM susceptible to cleavage by reducing agent, the AAs can be used to detect (either qualitatively or quantitatively) the presence of reducing conditions in a sample. To facilitate analysis in these methods, the AAs can be detectably labeled, and can be bound to a support (e.g., a solid support, such as a slide or bead). The detectable label can be positioned on a portion of the AA that is not released following cleavage, for example, the detectable label can be a quenched fluorescent label or other label that is not detectable until cleavage has occurred. The assay can be conducted by, for example, contacting the immobilized, detectably labeled AAs with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the AAs prior to contacting with the sample e.g., the presence of and/or an increase in detectable signal due to cleavage of the AA by the cleaving agent in the sample.

Such detection methods can be adapted to also provide for detection of the presence or absence of a target that is capable of binding the AB of the AAs when cleaved. Thus, the assays can be adapted to assess the presence or absence of a cleaving agent and the presence or absence of a target of interest. The presence or absence of the cleaving agent can be detected by the presence of and/or an increase in detectable label of the AAs as described above, and the presence or absence of the target can be detected by detection of a target-AB complex e.g., by use of a detectably labeled anti-target antibody.

AAs are also useful in in situ imaging for the validation of AA activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an AA is labeled with a detectable label. The detectable label may be a fluorescent dye, (e.g. a fluorophore, Fluorescein Isothiocyanate (FITC), Rhodamine Isothiocyanate (TRITC), an Alexa Fluor® label), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, AA indicates that the sample contains the target and contains a protease that is specific for the CM of the activatable antibody. In some embodiments, the presence of the protease can be confirmed using broad spectrum protease inhibitors such as those described herein, and/or by using an agent that is specific for the protease, for example, an antibody such as All, which is specific for the protease matriptase and inhibits the proteolytic activity of matriptase; see e.g., International Publication Number WO 2010/129609, published 11 Nov. 2010. The same approach of using broad spectrum protease inhibitors such as those described herein, and/or by using a more selective inhibitory agent can be used to identify a protease that is specific for the CM of the activatable antibody. In some embodiments, the presence of the target can be confirmed using an agent that is specific for the target, e.g., another antibody, or the detectable label can be competed with unlabeled target. In some embodiments, unlabeled AA could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the activatable antibody.

These techniques are also useful in kits and/or as reagents for the detection, identification or characterization of protease activity in a variety of cells, tissues, and organisms based on the protease-specific CM in the activatable antibody.

The disclosure provides methods of using the AAs in a variety of diagnostic and/or prophylactic indications. For example, the disclosure provides methods of detecting presence or absence of a cleaving agent and a target of interest in a subject or a sample by (i) contacting a subject or sample with an activatable antibody, wherein the AA comprises a masking moiety (MM), a cleavable moiety (CM) that is cleaved by the cleaving agent, e.g., a protease, and an antigen binding domain or fragment thereof (AB) that specifically binds the target of interest, wherein the AA in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MM does not interfere or compete with specific binding of the AB to the target; and (ii) measuring a level of activated AA in the subject or sample, wherein a detectable level of activated AA in the subject or sample indicates that the cleaving agent and the target are present in the subject or sample and wherein no detectable level of activated AA in the subject or sample indicates that the cleaving agent, the target or both the cleaving agent and the target are absent and/or not sufficiently present in the subject or sample. In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

The disclosure also provides methods of detecting presence or absence of a cleaving agent in a subject or a sample by (i) contacting a subject or sample with an AA in the presence of a target of interest, e.g., the target, wherein the AA comprises a masking moiety (MM), a cleavable moiety (CM) that is cleaved by the cleaving agent, e.g., a protease, and an antigen binding domain or fragment thereof (AB) that specifically binds the target of interest, wherein the AA in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MM does not interfere or compete with specific binding of the AB to the target; and (ii) measuring a level of activated AA in the subject or sample, wherein a detectable level of activated AA in the subject or sample indicates that the cleaving agent is present in the subject or sample and wherein no detectable level of activated AA in the subject or sample indicates that the cleaving agent is absent and/or not sufficiently present in the subject or sample. In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an AA comprises a masking moiety (MM), a cleavable moiety (CM) that is cleaved by the cleaving agent, e.g., a protease, and an antigen binding domain or fragment thereof (AB) that specifically binds the target of interest, wherein the AA in an uncleaved, non-activated state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB and is not a modified form of a natural binding partner of the AB; and (b) wherein, in an uncleaved, non-activated state, the MM interferes with specific binding of the AB to the target, and in a cleaved, activated state the MM does not interfere or compete with specific binding of the AB to the target; and (ii) measuring a level of activated AA in the subject or sample, wherein a detectable level of activated AA in the subject or sample indicates that the cleaving agent is present in the subject or sample and wherein no detectable level of activated AA in the subject or sample indicates that the cleaving agent is absent and/or not sufficiently present in the subject or sample. In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

The disclosure also provides methods of detecting presence or absence of a cleaving agent in a subject or a sample by (i) contacting a subject or sample with an activatable antibody, wherein the AA comprises a masking moiety (MM), a cleavable moiety (CM) that is cleaved by the cleaving agent, e.g., a protease, an antigen binding domain (AB) that specifically binds the target, and a detectable label, wherein the AA in an uncleaved, non-activated state comprises a structural arrangement from N-terminus to C-terminus as follows: MM-CM-AB or AB-CM-MM; wherein the MM is a peptide that inhibits binding of the AB to the target, and wherein the MM does not have an amino acid sequence of a naturally occurring binding partner of the AB and is not a modified form of a natural binding partner of the AB; wherein, in an uncleaved, non-activated state, the MM interferes with specific binding of the AB to the target, and in a cleaved, activated state the MM does not interfere or compete with specific binding of the AB to the target; and wherein the detectable label is positioned on a portion of the AA that is released following cleavage of the CM; and (ii) measuring a level of detectable label in the subject or sample, wherein a detectable level of the detectable label in the subject or sample indicates that the cleaving agent is absent and/or not sufficiently present in the subject or sample and wherein no detectable level of the detectable label in the subject or sample indicates that the cleaving agent is present in the subject or sample. In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an AA and/or conjugated AA (e.g., an AA to which a therapeutic agent is conjugated) described herein for use in contacting a subject or biological sample and means for detecting the level of activated AA and/or conjugated AA in the subject or biological sample, wherein a detectable level of activated AA in the subject or biological sample indicates that the cleaving agent and the target are present in the subject or biological sample and wherein no detectable level of activated AA in the subject or biological sample indicates that the cleaving agent, the target or both the cleaving agent and the target are absent and/or not sufficiently present in the subject or biological sample, such that the target binding and/or protease cleavage of the AA cannot be detected in the subject or biological sample.

The disclosure also provides methods of detecting presence or absence of a cleaving agent in a subject or a sample by (i) contacting a subject or biological sample with an AA in the presence of the target, and (ii) measuring a level of activated AA in the subject or biological sample, wherein a detectable level of activated AA in the subject or biological sample indicates that the cleaving agent is present in the subject or biological sample and wherein no detectable level of activated AA in the subject or biological sample indicates that the cleaving agent is absent and/or not sufficiently present in the subject or biological sample at a detectable level, such that protease cleavage of the AA cannot be detected in the subject or biological sample. Such an AA includes a masking moiety (MM), a cleavable moiety (CM) that is cleaved by the cleaving agent, e.g., a protease, and an antigen binding domain or fragment thereof (AB) that specifically binds the target, wherein the AA in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the AA in an uncleaved state interferes with specific binding of the AB to the target, and wherein the MM of an AA in a cleaved (i.e., activated) state does not interfere or compete with specific binding of the AB to the target. In some embodiments, the AA is an AA to which a therapeutic agent is conjugated. In some embodiments, the AA is not conjugated to an agent. In some embodiments, the detectable label is attached to the masking moiety. In some embodiments, the detectable label is attached to the CM N-terminal to the protease cleavage site. In some embodiments, a single antigen binding site of the AB is masked. In some embodiments wherein an antibody of the disclosure has at least two antigen binding sites, at least one antigen binding site is masked and at least one antigen binding site is not masked. In some embodiments all antigen binding sites are masked. In some embodiments, the measuring step includes use of a secondary reagent comprising a detectable label.

The disclosure also provides kits for use in methods of detecting presence or absence of a cleaving agent and the target in a subject or a sample, where the kits include at least an AA and/or conjugated AA described herein for use in contacting a subject or biological sample with an AA in the presence of the target, and measuring a level of activated AA in the subject or biological sample, wherein a detectable level of activated AA in the subject or biological sample indicates that the cleaving agent is present in the subject or biological sample and wherein no detectable level of activated AA in the subject or biological sample indicates that the cleaving agent is absent and/or not sufficiently present in the subject or biological sample at a detectable level, such that protease cleavage of the AA cannot be detected in the subject or biological sample. Such an AA includes a masking moiety (MM), a cleavable moiety (CM) that is cleaved by the cleaving agent, e.g., a protease, and an antigen binding domain or fragment thereof (AB) that specifically binds the target, wherein the AA in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the AA in an uncleaved state interferes with specific binding of the AB to the target, and wherein the MM of an AA in a cleaved (i.e., activated) state does not interfere or compete with specific binding of the AB to the target. In some embodiments, the AA is an AA to which a therapeutic agent is conjugated. In some embodiments, the AA is not conjugated to an agent. In some embodiments, the detectable label is attached to the masking moiety. In some embodiments, the detectable label is attached to the CM N-terminal to the protease cleavage site. In some embodiments, a single antigen binding site of the AB is masked. In some embodiments wherein an antibody of the disclosure has at least two antigen binding sites, at least one antigen binding site is masked and at least one antigen binding site is not masked. In some embodiments all antigen binding sites are masked. In some embodiments, the measuring step includes use of a secondary reagent comprising a detectable label.

The disclosure also provides kits for use in methods of detecting presence or absence of a cleaving agent in a subject or a sample, where the kits include at least an AA and/or conjugated AA described herein for use in contacting a subject or biological sample and means for detecting the level of activated AA and/or conjugated AA in the subject or biological sample, wherein the AA includes a detectable label that is positioned on a portion of the AA that is released following cleavage of the CM, wherein a detectable level of activated AA in the subject or biological sample indicates that the cleaving agent is absent and/or not sufficiently present in the subject or biological sample such that the target binding and/or protease cleavage of the AA cannot be detected in the subject or biological sample, and wherein no detectable level of activated AA in the subject or biological sample indicates that the cleaving agent is present in the subject or biological sample at a detectable level.

The disclosure provides methods of detecting presence or absence of a cleaving agent and the target in a subject or a sample by (i) contacting a subject or biological sample with an activatable antibody, wherein the AA includes a detectable label that is positioned on a portion of the AA that is released following cleavage of the CM and (ii) measuring a level of activated AA in the subject or biological sample, wherein a detectable level of activated AA in the subject or biological sample indicates that the cleaving agent, the target or both the cleaving agent and the target are absent and/or not sufficiently present in the subject or biological sample, such that the target binding and/or protease cleavage of the AA cannot be detected in the subject or biological sample, and wherein a reduced detectable level of activated AA in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%. Such an AA includes a masking moiety (MM), a cleavable moiety (CM) that is cleaved by the cleaving agent, and an antigen binding domain or fragment thereof (AB) that specifically binds the target, wherein the AA in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the AA in an uncleaved state interferes with specific binding of the AB to the target, and wherein the MM of an AA in a cleaved (i.e., activated) state does not interfere or compete with specific binding of the AB to the target. In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent and the target in a subject or a sample, where the kits include at least an AA and/or conjugated AA described herein for use in contacting a subject or biological sample and means for detecting the level of activated AA and/or conjugated AA in the subject or biological sample, wherein a detectable level of activated AA in the subject or biological sample indicates that the cleaving agent, the target or both the cleaving agent and the target are absent and/or not sufficiently present in the subject or biological sample, such that the target binding and/or protease cleavage of the AA cannot be detected in the subject or biological sample, and wherein a reduced detectable level of activated AA in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%.

The disclosure also provides methods of detecting presence or absence of a cleaving agent in a subject or a sample by (i) contacting a subject or biological sample with an activatable antibody, wherein the AA includes a detectable label that is positioned on a portion of the AA that is released following cleavage of the CM; and (ii) measuring a level of detectable label in the subject or biological sample, wherein a detectable level of the detectable label in the subject or biological sample indicates that the cleaving agent is absent and/or not sufficiently present in the subject or biological sample at a detectable level, such that protease cleavage of the AA cannot be detected in the subject or biological sample, and wherein a reduced detectable level of the detectable label in the subject or biological sample indicates that the cleaving agent is present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%. Such an AA includes a masking moiety (MM), a cleavable moiety (CM) that is cleaved by the cleaving agent, and an antigen binding domain or fragment thereof (AB) that specifically binds the target, wherein the AA in an uncleaved (i.e., non-activated) state comprises a structural arrangement from N-terminus to C-terminus as follows: MM-CM-AB or AB-CM-MM; (a) wherein the MM is a peptide that inhibits binding of the AB to the target, and wherein the MM does not have an amino acid sequence of a naturally occurring binding partner of the AB; and (b) wherein the MM of the AA in an uncleaved state interferes with specific binding of the AB to the target, and wherein the MM of an AA in a cleaved (i.e., activated) state does not interfere or compete with specific binding of the AB to the target. In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

The disclosure also provides kits for use in methods of detecting presence or absence of a cleaving agent of interest in a subject or a sample, where the kits include at least an AA and/or conjugated AA described herein for use in contacting a subject or biological sample and means for detecting the level of activated AA and/or conjugated AA in the subject or biological sample, wherein the AA includes a detectable label that is positioned on a portion of the AA that is released following cleavage of the CM, wherein a detectable level of the detectable label in the subject or biological sample indicates that the cleaving agent, the target, or both the cleaving agent and the target are absent and/or not sufficiently present in the subject or biological sample, such that the target binding and/or protease cleavage of the AA cannot be detected in the subject or biological sample, and wherein a reduced detectable level of the detectable label in the subject or biological sample indicates that the cleaving agent and the target are present in the subject or biological sample. A reduced level of detectable label is, for example, a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% and/or about 100%.

In some embodiments of these methods and kits, the AA includes a detectable label. In some embodiments of these methods and kits, the detectable label includes an imaging agent, a contrasting agent, an enzyme, a fluorescent label, a chromophore, a dye, one or more metal ions, or a ligand-based label. In some embodiments of these methods and kits, the imaging agent comprises a radioisotope. In some embodiments of these methods and kits, the radioisotope is indium or technetium. In some embodiments of these methods and kits, the contrasting agent comprises iodine, gadolinium or iron oxide. In some embodiments of these methods and kits, the enzyme comprises horseradish peroxidase, alkaline phosphatase, or β-galactosidase. In some embodiments of these methods and kits, the fluorescent label comprises yellow fluorescent protein (YFP), cyan fluorescent protein (CFP), green fluorescent protein (GFP), modified red fluorescent protein (mRFP), red fluorescent protein tdimer2 (RFP tdimer2), HCRED, or a europium derivative. In some embodiments of these methods and kits, the luminescent label comprises an N-methylacrydium derivative. In some embodiments of these methods, the label comprises an Alexa Fluor® label, such as Alex Fluor® 680 or Alexa Fluor® 750. In some embodiments of these methods and kits, the ligand-based label comprises biotin, avidin, streptavidin or one or more haptens.

In some embodiments of these methods and kits, the subject is a mammal. In some embodiments of these methods and kits, the subject is a human. In some embodiments, the subject is a non-human mammal, such as a non-human primate, companion animal (e.g., cat, dog, horse), farm animal, work animal, or zoo animal. In some embodiments, the subject is a rodent.

In some embodiments of these methods, the method is an in vivo method. In some embodiments of these methods, the method is an in situ method. In some embodiments of these methods, the method is an ex vivo method. In some embodiments of these methods, the method is an in vitro method.

In some embodiments, in situ imaging and/or in vivo imaging are useful in methods to identify which subjects to treat. For example, in in situ imaging, the AAs are used to screen subject samples to identify those subjects having the appropriate protease(s) and target(s) at the appropriate location, e.g., at a tumor site.

In some embodiments in situ imaging is used to identify or otherwise refine a subject population suitable for treatment with an AA of the disclosure. For example, subjects that test positive for both the target (e.g., the target) and a protease that cleaves the substrate in the CM (CM) of the AA being tested (e.g., accumulate activated antibodies at the disease site) are identified as suitable candidates for treatment with such an AA comprising such a CM. Likewise, subjects that test negative for either or both of the target (e.g., the target) and the protease that cleaves the substrate in the CM in the AA being tested using these methods might be identified as suitable candidates for another form of therapy. In some embodiments, such subjects that test negative with respect to a first AA can be tested with other AAs comprising different CMs until a suitable AA for treatment is identified (e.g., an AA comprising a CM that is cleaved by the subject at the site of disease). In some embodiments, the subject is then administered a therapeutically effective amount of the AA for which the subject tested positive.

In some embodiments in vivo imaging is used to identify or otherwise refine a subject population suitable for treatment with an AA of the disclosure. For example, subjects that test positive for both the target (e.g., the target) and a protease that cleaves the substrate in the CM (CM) of the AA being tested (e.g., accumulate activated antibodies at the disease site) are identified as suitable candidates for treatment with such an AA comprising such a CM. Likewise, subjects that test negative might be identified as suitable candidates for another form of therapy. In some embodiments, such subjects that test negative with respect to a first AA can be tested with other AAs comprising different CMs until a suitable AA for treatment is identified (e.g., an AA comprising a CM that is cleaved by the subject at the site of disease). In some embodiments, the subject is then administered a therapeutically effective amount of the AA for which the subject tested positive.

In some embodiments of the methods and kits, the method or kit is used to identify or otherwise refine a subject population suitable for treatment with an AA of the disclosure. For example, subjects that test positive for both the target (e.g., the target) and a protease that cleaves the substrate in the CM (CM) of the AA being tested in these methods are identified as suitable candidates for treatment with such an AA comprising such a CM. Likewise, subjects that test negative for both of the targets (e.g., the target) and the protease that cleaves the substrate in the CM in the AA being tested using these methods might be identified as suitable candidates for another form of therapy. In some embodiments, such subjects can be tested with other AAs until a suitable AA for treatment is identified (e.g., an AA comprising a CM that is cleaved by the subject at the site of disease). In some embodiments, subjects that test negative for either of the target (e.g., the target) are identified as suitable candidates for treatment with such an AA comprising such a CM. In some embodiments, subjects that test negative for either of the target (e.g., the target) are identified as not being suitable candidates for treatment with such an AA comprising such a CM. In some embodiments, such subjects can be tested with other AAs until a suitable AA for treatment is identified (e.g., an AA comprising a CM that is cleaved by the subject at the site of disease). In some embodiments, the AA is an AA to which a therapeutic agent is conjugated. In some embodiments, the AA is not conjugated to an agent. In some embodiments, the AA comprises a detectable label. In some embodiments, the detectable label is positioned on the AB. In some embodiments, measuring the level of AA in the subject or sample is accomplished using a secondary reagent that specifically binds to the activated antibody, wherein the reagent comprises a detectable label. In some embodiments, the secondary reagent is an antibody comprising a detectable label.

In some embodiments, a method or kit is used to identify or otherwise refine a subject population suitable for treatment with an anti-the target AA and/or conjugated AA (e.g., AA to which a therapeutic agent is conjugated) of the disclosure, followed by treatment by administering that AA and/or conjugated AA to a subject in need thereof. For example, subjects that test positive for both the targets (e.g., the target) and a protease that cleaves the substrate in the CM (CM) of the AA and/or conjugated AA being tested in these methods are identified as suitable candidates for treatment with such antibody and/or such a conjugated AA comprising such a CM, and the subject is then administered a therapeutically effective amount of the AA and/or conjugated AA that was tested. Likewise, subjects that test negative for either or both of the target (e.g., the target) and the protease that cleaves the substrate in the CM in the AA being tested using these methods might be identified as suitable candidates for another form of therapy. In some embodiments, such subjects can be tested with other antibody and/or conjugated AA until a suitable antibody and/or conjugated AA for treatment is identified (e.g., an AA and/or conjugated AA comprising a CM that is cleaved by the subject at the site of disease). In some embodiments, the subject is then administered a therapeutically effective amount of the AA and/or conjugated AA for which the subject tested positive.

In some embodiments of these methods and kits, the MM is a peptide having a length from about 4 to 40 amino acids. In some embodiments of these methods and kits, the AA comprises a linker peptide, wherein the linker peptide is positioned between the MM and the CM. In some embodiments of these methods and kits, the AA comprises a linker peptide, where the linker peptide is positioned between the AB and the CM. In some embodiments of these methods and kits, the AA comprises a first linker peptide (LP1) and a second linker peptide (LP2), wherein the first linker peptide is positioned between the MM and the CM and the second linker peptide is positioned between the AB and the CM. In some embodiments of these methods and kits, each of LP1 and LP2 is a peptide of about 1 to 20 amino acids in length, and wherein each of LP1 and LP2 need not be the same linker. In some embodiments of these methods and kits, one or both of LP1 and LP2 comprises a glycine-serine polymer. In some embodiments of these methods and kits, at least one of LP1 and LP2 comprises an amino acid sequence selected from the group consisting of (GS)n, (GSGGS)n (SEQ ID NO: 1) and (GGGS)n (SEQ ID NO: 2), where n is an integer of at least one. In some embodiments of these methods and kits, at least one of LP1 and LP2 comprises an amino acid sequence having the formula (GGS)n, where n is an integer of at least one. In some embodiments of these methods and kits, at least one of LP1 and LP2 comprises an amino acid sequence selected from the group consisting of Gly-Gly-Ser-Gly (SEQ ID NO: 3), Gly-Gly-Ser-Gly-Gly (SEQ ID NO: 4), Gly-Ser-Gly-Ser-Gly (SEQ ID NO: 5), Gly-Ser-Gly-Gly-Gly (SEQ ID NO: 6), Gly-Gly-Gly-Ser-Gly (SEQ ID NO: 7), and Gly-Ser-Ser-Ser-Gly (SEQ ID NO: 8).

In some embodiments of these methods and kits, the AB comprises an antibody or antibody fragment sequence selected from the cross-reactive antibody sequences presented herein. In some embodiments of these methods and kits, the AB comprises a Fab fragment, a scFv or a single chain antibody (scAb).

In some embodiments of these methods and kits, the cleaving agent is a protease that is co-localized in the subject or sample with the target and the CM is a polypeptide that functions as a substrate for the protease, wherein the protease cleaves the CM in the AA when the AA is exposed to the protease. In some embodiments of these methods and kits, the CM is a polypeptide of up to 15 amino acids in length. In some embodiments of these methods and kits, the CM is coupled to the N-terminus of the AB. In some embodiments of these methods and kits, the CM is coupled to the C-terminus of the AB. In some embodiments of these methods and kits, the CM is coupled to the N-terminus of a VL chain of the AB.

The antibodies, conjugated antibodies, AAs and conjugated AAs of the disclosure are used in diagnostic and prophylactic formulations. In one embodiment, an AA is administered to subjects that are at risk of developing one or more of the aforementioned inflammations, inflammatory disorders, cancer or other disorders.

A subject's or organ's predisposition to one or more of the aforementioned disorders can be determined using genotypic, serological or biochemical markers.

In some embodiments of the disclosure, an AA and/or a conjugated AA is administered to human individuals diagnosed with a clinical indication associated with one or more of the aforementioned disorders. Upon diagnosis, an AA and/or a conjugated AA is administered to mitigate or reverse the effects of the clinical indication.

Antibodies, conjugated antibodies, AAs and conjugated AAs of the disclosure are also useful in the detection of the target in subject samples and accordingly are useful as diagnostics. For example, the antibodies, conjugated antibodies, the AAs and conjugated AAs of the disclosure are used in in vitro assays, e.g., ELISA, to detect target levels in a subject sample.

In one embodiment, an antibody and/or AA of the disclosure is immobilized on a solid support (e.g., the well(s) of a microtiter plate). The immobilized antibody and/or AA serves as a capture antibody for any target that may be present in a test sample. Prior to contacting the immobilized antibody and/or AA with a subject sample, the solid support is rinsed and treated with a blocking agent such as milk protein or albumin to prevent nonspecific adsorption of the analyte.

Subsequently the wells are treated with a test sample suspected of containing the antigen, or with a solution containing a standard amount of the antigen. Such a sample is, e.g., a serum sample from a subject suspected of having levels of circulating antigen considered to be diagnostic of a pathology. After rinsing away the test sample or standard, the solid support is treated with a second antibody that is detectably labeled. The labeled second antibody serves as a detecting antibody. The level of detectable label is measured, and the concentration of target antigen in the test sample is determined by comparison with a standard curve developed from the standard samples.

It will be appreciated that based on the results obtained using the antibodies and/or AAs of the disclosure in an in vitro diagnostic assay, it is possible to stage a disease in a subject based on expression levels of the Target antigen. For a given disease, samples of blood are taken from subjects diagnosed as being at various stages in the progression of the disease, and/or at various points in the therapeutic treatment of the disease. Using a population of samples that provides statistically significant results for each stage of progression or therapy, a range of concentrations of the antigen that may be considered characteristic of each stage is designated.

Antibodies, conjugated antibodies, AAs and conjugated AAs can also be used in diagnostic and/or imaging methods. In some embodiments, such methods are in vitro methods. In some embodiments, such methods are in vivo methods. In some embodiments, such methods are in situ methods. In some embodiments, such methods are ex vivo methods. For example, AAs having an enzymatically cleavable CM can be used to detect the presence or absence of an enzyme that is capable of cleaving the CM. Such AAs can be used in diagnostics, which can include in vivo detection (e.g., qualitative or quantitative) of enzyme activity (or, in some embodiments, an environment of increased reduction potential such as that which can provide for reduction of a disulfide bond) through measured accumulation of activated antibodies (i.e., antibodies resulting from cleavage of an activatable antibody) in a given cell or tissue of a given host organism. Such accumulation of activated antibodies indicates not only that the tissue expresses enzymatic activity (or an increased reduction potential depending on the nature of the CM) but also that the tissue expresses target to which the activated antibody binds.

For example, the CM can be selected to be a protease substrate for a protease found at the site of a tumor, at the site of a viral or bacterial infection at a biologically confined site (e.g., such as in an abscess, in an organ, and the like), and the like. The AB can be one that binds a target antigen. Using methods familiar to one skilled in the art, a detectable label (e.g., a fluorescent label or radioactive label or radiotracer) can be conjugated to an AB or other region of an activatable antibody. Suitable detectable labels are discussed in the context of the above screening methods and additional specific examples are provided below. Using an AB specific to a protein or peptide of the disease state, along with a protease whose activity is elevated in the disease tissue of interest, AAs will exhibit an increased rate of binding to disease tissue relative to tissues where the CM specific enzyme is not present at a detectable level or is present at a lower level than in disease tissue or is inactive (e.g., in zymogen form or in complex with an inhibitor). Since small proteins and peptides are rapidly cleared from the blood by the renal filtration system, and because the enzyme specific for the CM is not present at a detectable level (or is present at lower levels in non-disease tissues or is present in inactive conformation), accumulation of activated antibodies in the disease tissue is enhanced relative to non-disease tissues.

In another example, AAs can be used to detect the presence or absence of a cleaving agent in a sample. For example, where the AAs contain a CM susceptible to cleavage by an enzyme, the AAs can be used to detect (either qualitatively or quantitatively) the presence of an enzyme in the sample. In another example, where the AAs contain a CM susceptible to cleavage by reducing agent, the AAs can be used to detect (either qualitatively or quantitatively) the presence of reducing conditions in a sample. To facilitate analysis in these methods, the AAs can be detectably labeled, and can be bound to a support (e.g., a solid support, such as a slide or bead). The detectable label can be positioned on a portion of the AA that is not released following cleavage, for example, the detectable label can be a quenched fluorescent label or other label that is not detectable until cleavage has occurred. The assay can be conducted by, for example, contacting the immobilized, detectably labeled AAs with a sample suspected of containing an enzyme and/or reducing agent for a time sufficient for cleavage to occur, then washing to remove excess sample and contaminants. The presence or absence of the cleaving agent (e.g., enzyme or reducing agent) in the sample is then assessed by a change in detectable signal of the AAs prior to contacting with the sample e.g., the presence of and/or an increase in detectable signal due to cleavage of the AA by the cleaving agent in the sample.

Such detection methods can be adapted to also provide for detection of the presence or absence of a target that is capable of binding the AB of the AAs when cleaved. Thus, the assays can be adapted to assess the presence or absence of a cleaving agent and the presence or absence of a target of interest. The presence or absence of the cleaving agent can be detected by the presence of and/or an increase in detectable label of the AAs as described above, and the presence or absence of the target can be detected by detection of a target-AB complex e.g., by use of a detectably labeled anti-target antibody.

AAs are also useful in in situ imaging for the validation of AA activation, e.g., by protease cleavage, and binding to a particular target. In situ imaging is a technique that enables localization of proteolytic activity and target in biological samples such as cell cultures or tissue sections. Using this technique, it is possible to confirm both binding to a given target and proteolytic activity based on the presence of a detectable label (e.g., a fluorescent label).

These techniques are useful with any frozen cells or tissue derived from a disease site (e.g. tumor tissue) or healthy tissues. These techniques are also useful with fresh cell or tissue samples.

In these techniques, an AA is labeled with a detectable label. The detectable label may be a fluorescent dye, (e.g. Fluorescein Isothiocyanate (FITC), Rhodamine Isothiocyanate (TRITC), a near infrared (NIR) dye (e.g., Qdot® nanocrystals), a colloidal metal, a hapten, a radioactive marker, biotin and an amplification reagent such as streptavidin, or an enzyme (e.g. horseradish peroxidase or alkaline phosphatase).

Detection of the label in a sample that has been incubated with the labeled, AA indicates that the sample contains the target and contains a protease that is specific for the CM of the activatable antibody. In some embodiments, the presence of the protease can be confirmed using broad spectrum protease inhibitors such as those described herein, and/or by using an agent that is specific for the protease, for example, an antibody such as All, which is specific for the protease matriptase and inhibits the proteolytic activity of matriptase; see e.g., International Publication Number WO 2010/129609, published 11 Nov. 2010. The same approach of using broad spectrum protease inhibitors such as those described herein, and/or by using a more selective inhibitory agent can be used to identify a protease or class of proteases specific for the CM of the activatable antibody. In some embodiments, the presence of the target can be confirmed using an agent that is specific for the target, e.g., another antibody, or the detectable label can be competed with unlabeled target. In some embodiments, unlabeled AA could be used, with detection by a labeled secondary antibody or more complex detection system.

Similar techniques are also useful for in vivo imaging where detection of the fluorescent signal in a subject, e.g., a mammal, including a human, indicates that the disease site contains the target and contains a protease that is specific for the CM of the activatable antibody.

These techniques are also useful in kits and/or as reagents for the detection, identification or characterization of protease activity in a variety of cells, tissues, and organisms based on the protease-specific CM in the activatable antibody.

In some embodiments, in situ imaging and/or in vivo imaging are useful in methods to identify which subjects to treat. For example, in in situ imaging, the AAs are used to screen subject samples to identify those subjects having the appropriate protease(s) and target(s) at the appropriate location, e.g., at a tumor site.

In some embodiments in situ imaging is used to identify or otherwise refine a subject population suitable for treatment with an AA of the disclosure. For example, subjects that test positive for both the target and a protease that cleaves the substrate in the CM (CM) of the AA being tested (e.g., accumulate activated antibodies at the disease site) are identified as suitable candidates for treatment with such an AA comprising such a CM. Likewise, subjects that test negative for either or both of the target and the protease that cleaves the substrate in the CM in the AA being tested using these methods are identified as suitable candidates for another form of therapy (i.e., not suitable for treatment with the AA being tested). In some embodiments, such subjects that test negative with respect to a first AA can be tested with other AAs comprising different CMs until a suitable AA for treatment is identified (e.g., an AA comprising a CM that is cleaved by the subject at the site of disease).

In some embodiments in vivo imaging is used to identify or otherwise refine a subject population suitable for treatment with an AA of the disclosure. For example, subjects that test positive for both the target and a protease that cleaves the substrate in the CM (CM) of the AA being tested (e.g., accumulate activated antibodies at the disease site) are identified as suitable candidates for treatment with such an AA comprising such a CM. Likewise, subjects that test negative are identified as suitable candidates for another form of therapy (i.e., not suitable for treatment with the AA being tested). In some embodiments, such subjects that test negative with respect to a first AA can be tested with other AAs comprising different CMs until a suitable AA for treatment is identified (e.g., an AA comprising a CM that is cleaved by the subject at the site of disease).

### Pharmaceutical Compositions

The AAs and conjugated AAs of the disclosure (also referred to herein as “active compounds”), and derivatives, fragments, analogs and homologs thereof, can be incorporated into pharmaceutical compositions suitable for administration. Such compositions typically comprise the AA and/or conjugated AA and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Suitable examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the disclosure is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. In an exemplary embodiment, the route of administration is intravenous.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL′ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In some embodiments, it will be desirable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

### Dosing

As provided herein, as subject is administered the AA or a conjugated AA at a dose of anywhere from about 1 ng/kg to 100 g/kg. In exemplary embodiments, the subject is administered the AA or the conjugated AA at a dose of greater than 6 mg/kg to about 10 mg/kg. In one embodiment, the subject is administered the AA or the conjugated AA at a dose of greater than 6 mg/kg. In another embodiment, the subject is administered the AA or the conjugated AA at a dose of about 7 mg/kg. In another embodiment, the subject is administered the AA or the conjugated AA at a dose of about 8 mg/kg. In another embodiment, the subject is administered the AA or the conjugated AA at a dose of about 9 mg/kg. In another embodiment, the subject is administered the AA or the conjugated AA at a dose of about 10 mg/kg. In another embodiment, the subject is administered the AA or the conjugated AA at a dose of greater than 6 mg/kg to about 7 mg/kg. In another embodiment, the subject is administered the AA or the conjugated AA at a dose of about 7 mg/kg to about 8 mg/kg. In another embodiment, the subject is administered the AA or the conjugated AA at a dose of about 8 mg/kg to about 9 mg/kg. In another embodiment, the subject is administered the AA or the conjugated AA at a dose of about 9 mg/kg to about 10 mg/kg. In another embodiment, the subject is administered the AA or the conjugated AA at a dose of greater than 6 mg/kg to about 8 mg/kg. In another embodiment, the subject is administered the AA or the conjugated AA at a dose of about 7 mg/kg to about 9 mg/kg. In another embodiment, the subject is administered the AA or the conjugated AA at a dose of about 8 mg/kg to about 10 mg/kg. In another embodiment, the subject is administered the AA or the conjugated AA at a fixed dose of greater than 240 mg to about 1000 mg. In another embodiment, the subject is administered the AA or the conjugated AA at a fixed dose of greater than 240 mg to about 400 mg. In another embodiment, the subject is administered the AA or the conjugated AA at a fixed dose of greater than 600 mg to about 1000 mg. In another embodiment, the subject is administered the AA or the conjugated AA at a fixed dose of greater than 240 mg to greater than 600 mg. In another embodiment, the subject is administered the AA or the conjugated AA at a fixed dose of greater than 240 mg to about 280 mg. In another embodiment, the subject is administered the AA or the conjugated AA at a fixed dose of about 280 mg to about 320 mg. In another embodiment, the subject is administered the AA or the conjugated AA at a fixed dose of about 320 mg to about 360 mg. In another embodiment, the subject is administered the AA or the conjugated AA at a fixed dose of about 360 mg to about 400 mg. In another embodiment, the subject is administered the AA or the conjugated AA at a fixed dose of greater than 240 mg to about 320 mg. In another embodiment, the subject is administered the AA or the conjugated AA at a fixed dose of about 280 mg to about 360 mg. In another embodiment, the subject is administered the AA or the conjugated AA at a fixed dose of about 320 mg to about 400 mg. In another embodiment, the subject is administered the AA or the conjugated AA at a fixed dose of greater than 600 mg to about 700 mg. In another embodiment, the subject is administered the AA or the conjugated AA at a fixed dose of about 700 mg to about 800 mg. In another embodiment, the subject is administered the AA or the conjugated AA at a fixed dose of about 800 mg to about 900 mg. In another embodiment, the subject is administered the AA or the conjugated AA at a fixed dose of about 900 mg to about 1000 mg. In another embodiment, the subject is administered the AA or the conjugated AA at a fixed dose of greater than 600 mg to about 800 mg. In another embodiment, the subject is administered the AA or the conjugated AA at a fixed dose of about 700 mg to about 900 mg. In another embodiment, the subject is administered the AA or the conjugated AA at a fixed dose of about 800 mg to about 1000 mg.

In some embodiments the subject is administered a conjugated AA based on the weight of the subject.

In some embodiments the subject is administered a conjugated AA in which the dosage when measured in mg/kg is based on the actual body weight of the subject.

In some embodiments the subject is administered a conjugated AA in which the dosage when measured in mg/kg is based on the adjusted ideal body weight (AIBW) of the subject. In some embodiments, the adjusted ideal body weight is calculated based on a difference between the given subject's actual body weight and a predetermined ideal body weight (IBW) for male and female subjects as corresponding to the subject. In some embodiments, the ideal body weight of the given subject is based on the height of the subject. In some embodiments, the ideal body weight (IBW) for a given male subject in kilograms is determined as IBW=0.9×(height in cm)−88, and the IBW for a given female subject in kilograms is determined as IBW=0.9×(height in cm)−92. In some embodiments, the adjusted ideal body weight (AIBW) for a given subject in kilograms is determined by AIBW=IBW+0.4×(actual weight−IBW), where the IBW is based on their given height and gender. In some embodiments, the male and female subjects are human subjects. In some embodiments, the AIBW of the human subjects are from about 40 kg to about 100 kg.

In some embodiments, the subject is administered the AA or the conjugated AA intravenously every day, every 2 days, every 3 days, every 4 days, every 5 days, every 6 days, every 7 days, every 8 days, every 9 days, every 10 days, every 11 days, every 12 days, every 13 days, every 14 days, every 15 days, every 16 days, every 17 days, every 18 days, every 19 days, every 20 days, every 21 days, or even every 30 days. In some embodiments, the subject is administered the AA or the conjugated AA intravenously for as long as the AA and/or agent is effective.

In some embodiments, the subject is administered the AA or the conjugated AA once daily. In some embodiments, the subject is administered the AA or the conjugated AA multiple times a day, for example every 4 hours, every 6 hours, every 4-6 hours, every 8 hours, or every 12 hours.

In some embodiments of the present disclosure, in conjunction with administration of the conjugated AA of the present disclosure, the subject can be treated prophylactically with one or more treatment regimens and/or precautions intended to mitigate or prevent ocular toxicity. Without being bound by theory, these prophylactic measures are intended to mitigate and/or prevent ocular toxicity associated with maytansinoids, such as the DM4 associated with the conjugated AAs of the present disclosure. Exemplary prophylactic measures to mitigate and/or prevent ocular toxicity include use of UV AB eye protection (e.g., sunglasses), use of artificial tear eye drops, topical vasoconstrictor eye drops (e.g., brimonidine tartrate ophthalmic solution, tetrahydrozoline eye drops), and/or topical steroid eye drops (e.g., prednisolone acetate eye drops). In some embodiments, administration of ocular prophylactic measures to the treated subjects is optional. In some embodiments, administration of ocular prophylactic measures the treated subjects is mandatory.

The invention will be further described in the following examples, which do not limit the scope of the invention described in the claims.

### EXAMPLES

### Example 1: Production and Testing of Conjugated Activatable Antibodies that Bind CD166

The AAs used in the example set forth below are provided herein and were generated and characterized using the methods disclosed in the PCT Publication No. WO 2016/179285, the contents of which are incorporated by reference herein in its entirety.

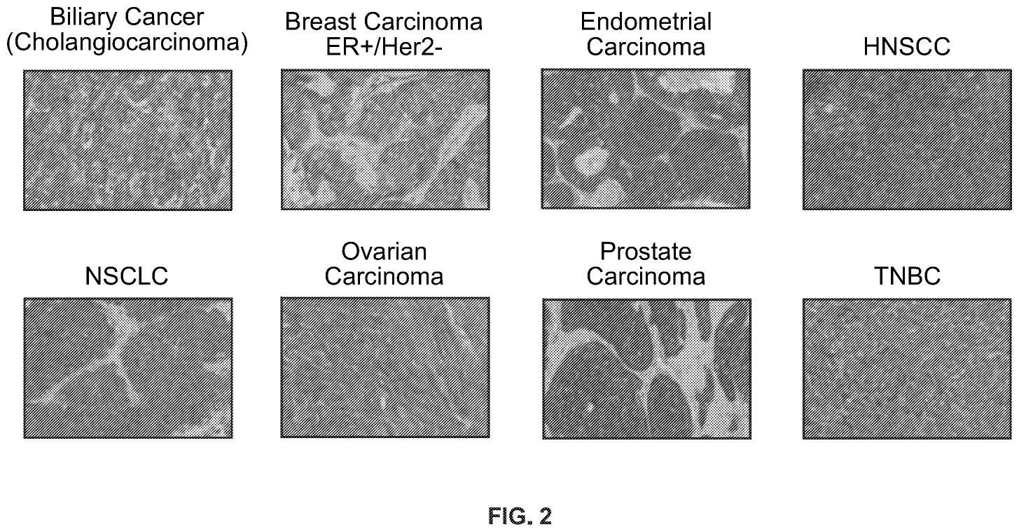

The activatable anti-CD166 antibody drug conjugates (AADC) (depicted in FIG. 1) demonstrate anti-tumor activity in mouse models with human xenograft tumors and are well-tolerated in preclinical studies (Weaver et al. AACR-NCI-EOTRC International Conference 2015). CD166 is widely expressed in many cancers and in healthy tissues as demonstrated in FIG. 2, Table 4A, Table 4B, and Table 5.

TABLE 4A

| | Prevalence | Prevalence | Prevalence | |
|---|---|---|---|---|
| Cancer type | of CD166 | of CD166 | of CD166- | Number |
| (commercial | expression | expression | negativity | of cases |
| samples) | (IHC 3+), % | (IHC ≥2+), % | (IHC <1+), % | examined |
| Biliary | Not | 56.5 | 11.9 | 177 |
| (cholangio- | calculated | | | |
| carcinoma) | | | | |
| Breast | 70% | 87.1 | 1.7 | 533 |
| Endometrial | 57% | 75.2 | 6.0 | 315 |
| Head and | 49% | 81.1 | 0.8 | 122 |
| neck | | | | |
| Lung | 60% | 71.0 | 8.2 | 465 |
| Prostate | 89% | 98.3 | 0.8 | 119 |
| Ovarian | 52% | 70.5 | 3.9 | 129 |

TABLE 4B

| | Prevalence of | Number |
|---|---|---|
| Cancer type | CD166 expression | of cases |
| (treated subjects) | (IHC 3+), % | examined |
| Breast | 79% | 95 |
| Endometrial | 67% | 3 |
| Head and neck | 62% | 21 |
| Lung | 64% | 22 |
| Prostate | 0% | 2 |
| Ovarian | 59% | 107 |

TABLE 5
CD166 expression in healthy human tissue by IHC

| | Human CD166 |
|---|---|
| Tissue Type | Expression |
| Adrenal Gland | −/+ |
| Bone Marrow | −/+ |
| Breast | +/++ |
| Brain, Cerebrum | −/+ |
| Brain, Cerebellum | −/+ |
| Cervix | +/++ |
| Colon | ++ |
| Esophagus | +/++ |
| Eye | + |
| Heart | + |
| Kidney | +/+++ |
| Larynx | +/++ |
| Liver | ++ |
| Lung | +/++ |
| Nerve | +/++ |
| Ovary | −/++ |
| Pancreas | ++/+++ |
| Prostate | ++/+++ |
| Skin | +/++ |
| Small Intestine | +/+++ |
| Spleen | +/++ |
| Stomach | +++ |
| Striated/Skeletal Muscle | −/+ |
| Testis | −/++ |
| Thyroid | ++/+++ |
| Thymus | + |
| Uterus | +/+++ |

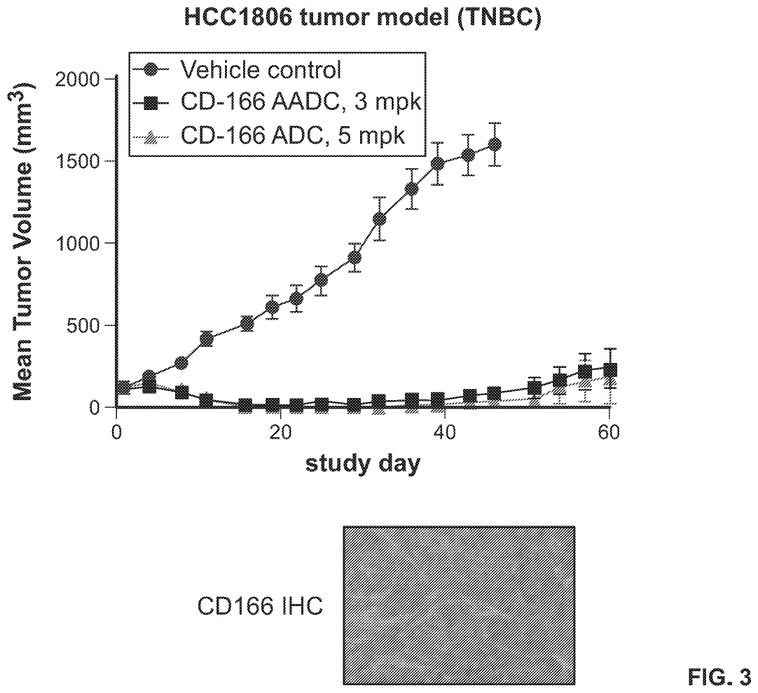

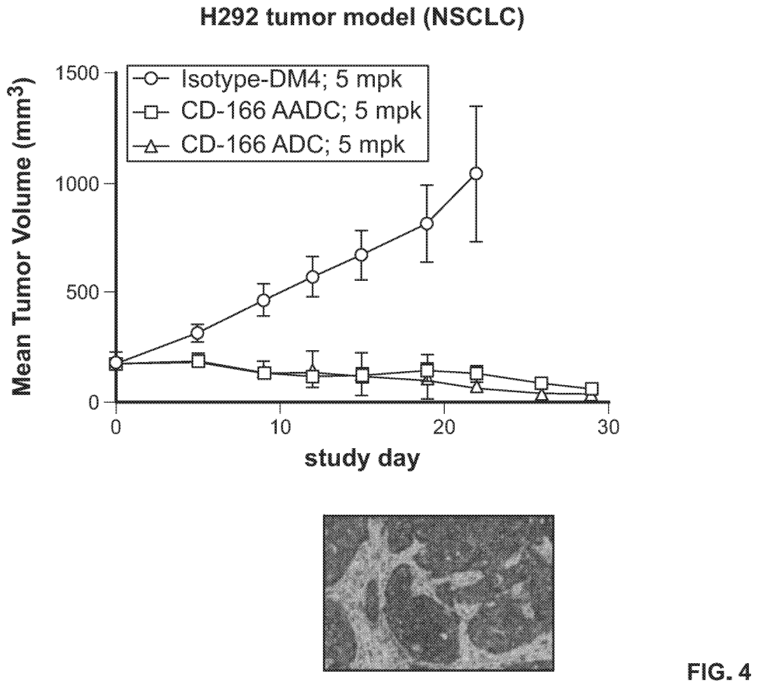

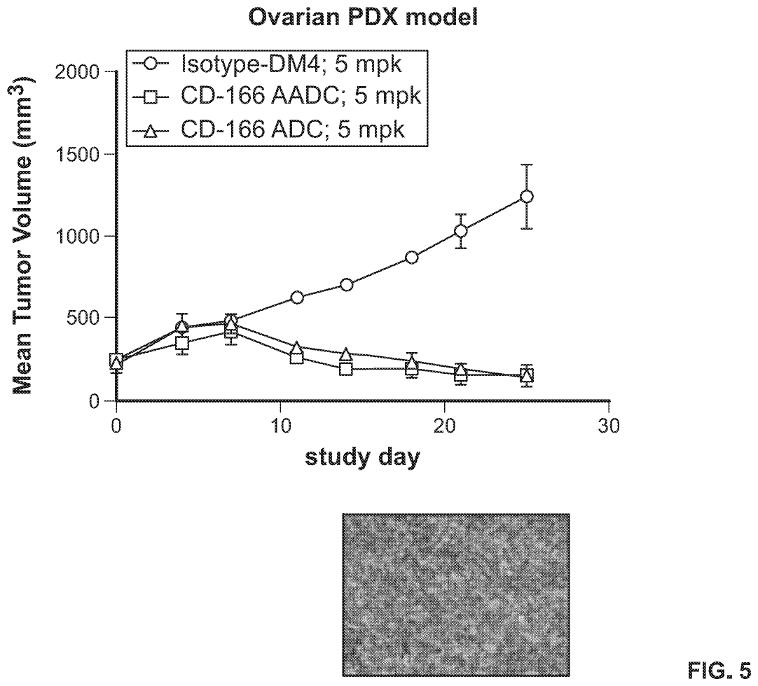

FIG. 3-6 show that the CD166 AA drug conjugates of the invention produced complete and durable responses in mouse models of human xenograft tumors at doses equal to or below the predicted human dose.

### Example 2: Open-Label, Multicenter, Dose-Escalation Study to Determine Safety of Activatable Anti-CD166 Antibody Drug Conjugates in Subjects with High CD-166 Expressing Tumors

In this study, the primary endpoints of safety, maximum tolerable dose (MTD), recommended phase 2 dose (RP2D), dose-limiting toxicities, and preliminary antitumor activity of activatable anti-CD166 antibody drug conjugates, administered as monotherapy in subjects with high CD166 expressing tumors (breast, lung, prostate, ovarian, endometrial, head and neck, and biliary carcinomas), are assessed.

Secondary end points include: (1) measuring objective response rate according to Response Evaluation in Criteria in Solid Tumors (RECIST) version 1.1 or tumor-specific criteria, as applicable; (2) time to response; (3) duration of response; (4) progression-free survival; (5) overall survival; (6) pharmacokinetic profile of AADCs including analyzing intact AADCs, total AADCs, total AADC-conjugated DM4, free DM4, and S-methyl DM4; and (7) incidence of anti-drug antibody formation.

Additional endpoints include (1) the identification of predictive biomarkers associated with the clinical activity of AADCs such as CD166 expression and mitotic markers (e.g. Ki-67) in tumor specimens prior to and while receiving treatment; and (2) characterization of the protease activity and activation of ADCCs in on-treatment tumor biopsy samples and peripheral blood, respectively.

The study presented in this example is an open-label, multicenter, dose-escalation, and proof-of-concept phase ½ study of anti-CD166 AADCs, wherein the anti-CD166 AADC comprises a DM4-conjugated activatable antibody of the anti-CD166 activatable antibody referred to herein as Combination 55, which comprises the heavy chain sequence of SEQ ID NO: 480 and the light chain sequence of SEQ ID NO: 246.

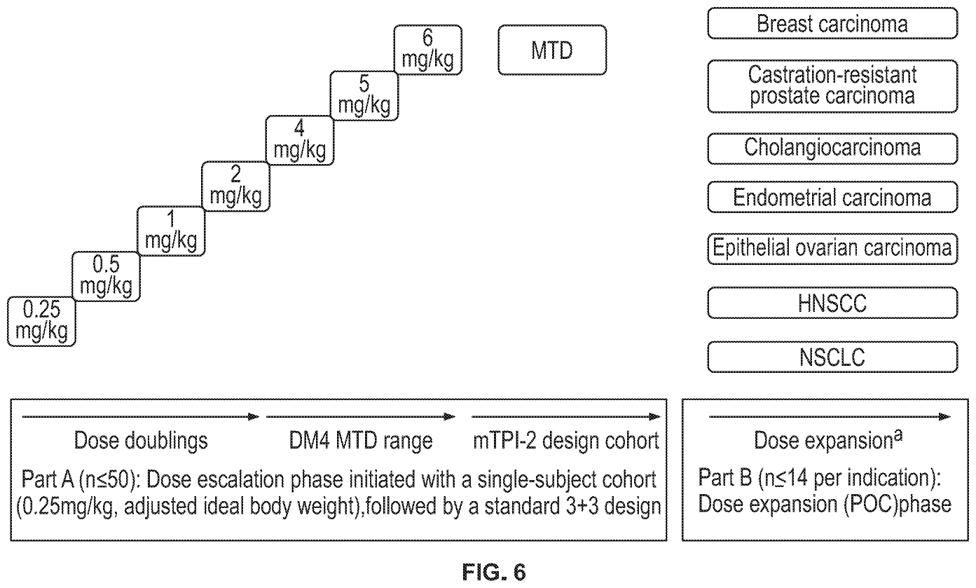

The study includes subjects with breast carcinoma, castration-resistant prostate cancer (CPRC), cholangiocarcinoma, endometrial carcinoma, epithelial ovarian carcinoma, head and neck squamous cell carcinoma (HNSCC), and non-small cell lung cancer (NSCLC). Subjects are treated with an activatable anti-CD166 antibody drug conjugate intravenously every 21 days, and the study proceeds in the following two parts, Part A and Part B. The study design is also depicted in FIG. 6.

In Part A (Dose Escalation) (n≤50), accelerated dose titration of the administered anti-CD166 ADCC is followed by a traditional 3+3 design. A 3+3 design is described as the following: 3 subjects are treated with a first dose of an anti-CD166 AADC and adverse effects noted. If no toxicity is observed, the dose is increased, and an additional three subjects are treated. If 1 of 3 subjects exhibits toxicity, 3 additional subjects are enrolled at the first dose. If 2 to 3 subjects show toxicity, that dose is denoted as the maximum tolerated dose (described in https://www.ncbi.nlm.nih.gov/pmc/articles/PMC2684552/). This study is performed to determine MTD and ends in a modified toxicity probability interval 2 (mTPI-2)-design cohort treated at the MTD to determine RP2D.

Part B (Dose Expansion) of the study is a dose expansion phase testing of the anti-CD166 AADC administered at the RP2D in the 7 tumor types (up to 14 subjects each, n≤98).

Subjects are treated until progression; duration of treatment is about 6 months with follow-up contact every 3 to 6 months or for another 1 or 2 years or as long as the subject is alive.

Up to 150 subjects are enrolled in the study in both the dose escalation and the expansion cohorts. Key eligibility criteria for the subjects are shown in Table 6A.

TABLE 6A

| | |
|---|---|
| Part A | Age ≥18 years |
| | Eastern cooperative Oncology Group (ECOG) performance |
| | status 0-1 |
| | Histologically confirmed diagnosis of any active metastatic |
| | or locally advanced unresectable solid tumor |
| | Agrees to provide tumor tissue (archival, new, or recent |
| | acquisition) prior to initiation of anti-CD166 AADC |
| | Life expectancy of ≥3 months |
| Part B | Consent from at least 7 subjects (at least 1 of each tumor |
| | type), to provide a baseline and an on-study tumor biopsy |
| | sample (if safe to perform biopsy) and peripheral blood sample |
| Breast carcinoma | Subjects with estrogen receptor expressing (ER+) breast |
| | carcinoma should have received anti-hormonal therapy and |
| | experienced disease progression |
| | TNBC received ≥2 previous lines of therapy |
| Castration-resistant | Received ≥1 prior therapy |
| prostate carcinoma | |
| Cholangiocarcinoma | Failed ≥1 prior line of gemcitabine-containing regimen |
| Endometrial carcinoma | Received ≥1 platinum-containing regimen for extra-uterine |
| | or advanced disease |
| Epithelial ovarian | Non-breast cancer (BRCA) mutation (germline or somatic) |
| carcinoma | subjects or subjects with unknown BRCA mutational status must |
| | have platinum-resistant or platinum refractory ovarian carcinoma |
| | Subjects with BRCA mutations must be refractory to or |
| | otherwise ineligible for PARP inhibitors |
| HNSCC | Received ≥1 platinum-containing regimen and PD-1/PD-L1 |
| | inhibitor, if approved for subject's indication and locality |
| NSCLC | Received ≥1 platinum containing regimen |
| | A checkpoint inhibitor should have been administered if |
| | approved for the subject's indication in their locality |
| Exclusion criteria | Active or chronic corneal disorder, history of corneal |
| | transplantation, active herpetic keratitis, and active ocular conditions |
| | requiring ongoing treatment/monitoring |
| | Serious concurrent illness, including clinically relevant |
| | active infection |
| | History of or current active autoimmune diseases |
| | Significant cardiac disease such as recent myocardial infarction |
| | History of multiple sclerosis or other demyelinating disease, |
| | Eaton-Lambert syndrome (para-neoplastic syndrome), history of |
| | hemorrhagic or ischemic stroke within the last 6 months, or |
| | alcoholic liver disease; |
| | Non-healing wound(s) or ulcer(s) except for ulcerative |
| | lesions caused by the underlying neoplasm; |
| | History of severe allergic or anaphylactic reactions to |
| | previous monoclonal antibody therapy; |
| | Currently receiving anticoagulation therapy with warfarin; |
| | Major surgery (requiring general anesthesia) within 3 months |
| | prior to dosing. |

Up to 150 subjects are enrolled in the study in both the dose escalation and the expansion cohorts. Adverse events and concomitant medications are assessed on day 1, day 8, and day 15 of anti-CD166 AADC cycle 1, followed by evaluations on the first day of each subsequent treatment cycle at the end of treatment. Assessment of ocular symptoms and ECOG performance score is performed at screening, the first day of each treatment cycle, and the end of the treatment. Complete ophthalmology examination is performed on all subjects at screening and during certain points of the study. Subjects who report treatment emergent changes in vision or other ocular symptoms will undergo repeat examinations prior to infusion in every other cycle and as clinically indicated. Hematology and serum chemistry are evaluated at every treatment visit. Archival tissue or fresh biopsy samples is provided at baseline for subjects participating in Part A. In Part B, pre- and on-treatment biopsies and the collection of peripheral blood samples (in part to determine the intactness of the activatable antibody) will be mandatory for at least 7 subjects, 1 of each tumor types. In some instances, biopsies from more than 1 subject of each tumor type is collected, for example biopsies from 2, 3, 4, 5, 6, 7, or more subjects are collected for each tumor type. Blood samples for pharmacokinetic, pharmacodynamics, and biomarker analyses is obtained at pre-specified time points. Imaging for tumor response assessment is performed, every 8 weeks from the first dose of the anti-CD166 AADC. After the last dose of study medication, subjects are evaluated every 3 months for the first year and then every 6 months or until death.

In this exemplary study, all patients undergo complete ophthalmology examination at baseline and during certain points of the study. In this exemplary study, patients who report treatment-emergent changes in vision or other ocular symptoms undergo repeat examinations before infusion in every other cycle and as clinically indicated.

At a point at which 78 patients were enrolled for treatment in the exemplary study, 58 of the patients (74%) were shown to have high CD166 expression, defined as 3+ membranous staining intensity in ≥50% of the tumor cells. This group of 78 patients had been treated with a median of 6 (ranging from 1-20) prior therapies, including anti-microtubule or platinum-containing agents (75/78 patients; 96%), and anti-PD-1 or anti-PD-L1 agents (25/78 patients; 32%).

At a given cut-off date during this exemplary study, 63/78 patients (81%) had discontinued treatment due to disease progression (35/78 patients; 45%), symptomatic deterioration (10/78 patients; 13%), adverse events related to study drug (9/78 patients; 12%), and investigator decision (3/78 patients; 4%), withdrawal by patient (3/78 patients; 4%), and death unrelated to study drug (3/78 patients; 4%). Up to this given cut-off date in the study, the patients had received a median of 2 doses of the drug (ranging from 1-13 doses) and the median treatment duration was 6.3 weeks (ranging from 0.3-42.1 weeks). A summary of the duration and administrated doses is shown in Table 6B.

TABLE 6B

| Dose | Median No. of | Median Treatment |
|---|---|---|
| (mg/kg) | Doses (range) | Duration, wks (range) |
| <4 | 3 | 8.9 |
| (n = 10) | (1-3) | (3.0-9.3) |
| 4-5 | 3 | 9.0 |
| (n = 19) | (1-13) | (3.0-42.1) |
| 6-7 | 2 | 6.2 |
| (n = 18) | (1-11) | (3.0-33.0) |
| 8-9 | 2 | 6.1 |
| (n = 23) | (1-7) | (0.3-22.1) |
| 10 | 2 | 6.0 |
| (n = 8) | (1-3) | (2.4-10.4) |
| All Cohorts | 2 | 6.3 |
| (n = 78) | (1-13) | (0.3-42.1) |

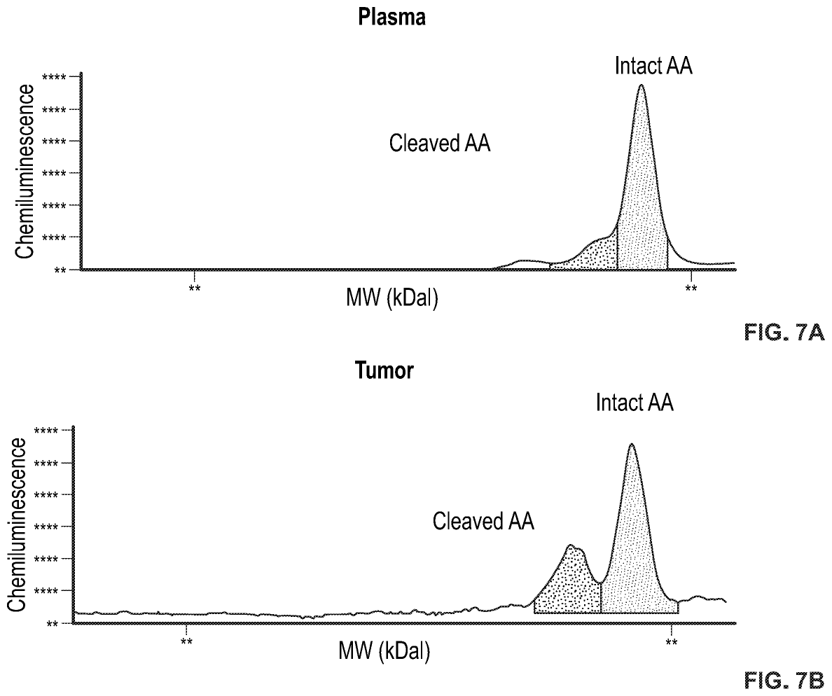

Several additional methods to evaluate drug-activatable anti-CD166 antibody drug conjugate activation and activity are listed in Table 7 and FIG. 7A, 7B.

TABLE 7

| Goal | Sample(s) | Assay | Method |
|---|---|---|---|
| Determine activation | Biopsy, | WES ™ | Capillary electrophoresis |
| of activatable anti- | plasma | assay | with immunodetection to |
| CD166 antibody drug | | | identify masked and |
| conjugate | | | activated AADC |
| | Biopsy | QZ ™ | Protease activity detection |
| | | assay | |
| Correlation of | Biopsy | IHC | CD166 expression, Ki-67 |
| markers with | | | |
| activatable anti- | | | |
| CD166 antibody drug | | | |
| conjugate activity | | | |

### Example 3. Quantification of Activated and Intact Anti-CD166 Activatable Antibodies in Biological Samples

This Example describes the ability to detect the activated and intact anti-CD166 activatable antibody 7614.6-3001-HuCD166 in plasma and xenograft tumor samples of mice administered 7614.6-3001-HuCD166.

The studies presented herein used the anti-CD166 activatable antibody referred to herein as 7614.6-3001-HuCD166, also referred to as HuCD166-7614.6-3001, which comprises the heavy chain sequence of SEQ ID NO: 480 and the light chain sequence of SEQ ID NO: 246.

Quantification of activated and intact anti-CD166 activatable antibody 7614.6-3001-HuCD166 was assessed by the Wes system using anti-human IgG antibodies (anti-human IgG(H&L), American Qualex Catalog #A110UK). Nude mice were implanted subcutaneously with 5×10e6 H292 cells in serum-free medium mixed 1:1 with Matrigel™. Mice harboring 200-500 mm2 H292 xenografts were dosed with 5 mg/kg of anti-CD166 activatable antibody 7614.6-3001-HuCD166. One day after treatment, tumor and plasma (heparin) were collected and stored at −80° C. prior to analysis. Tumor homogenates were prepared in Thermo Scientific Pierce™ IP Lysis Buffer (Catalog #87788) with added Thermo Scientific Halt™ Protease Inhibitor Single Use Cocktail Kit (Catalog #78430) using Barocycler (Pressure Biosciences). One mg/mL of protein lysate in IP lysis buffer with HALT protease inhibitor/EDTA and plasma samples diluted 1 in 20 in PBS were analyzed by the Wes system as described herein. FIG. 7A and FIG. 7B demonstrate preferential activation in tumor (FIG. 7B) as compared to plasma (FIG. 7A).

### Example 4. Quantification of Activated and Intact Anti-CD166 Conjugated Activatable Antibodies in Biological Samples

This Example describes the ability to detect activated and intact anti-CD166 activatable antibody, conjugated to maytansinoid toxin DM4 through an SPDB linker (Combination 55).

The example used a DM4-conjugated activatable antibody of the anti-CD166 activatable antibody referred to herein as Combination 55, which comprises the heavy chain sequence of SEQ ID NO: 480 and the light chain sequence of SEQ ID NO: 246 conjugated to DM4 via a spdb linker.

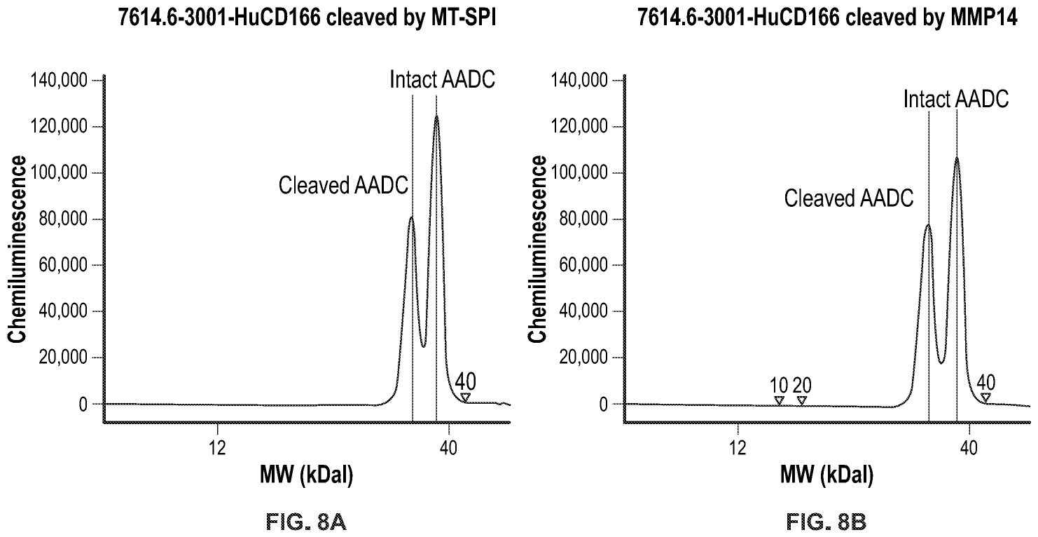

The anti-CD166 conjugated activatable antibody was activated with either 80 ug/ml of matriptase (R&D Systems Catalog #3946-SE) or 80 ug/ml of MMP14 (R&D Systems Catalog #918-MP) for 2 hours at 37C and mixed with intact conjugated activatable antibody. The mixture was then analyzed by the Wes system as described above using anti-human IgG (H&L) (American Qualex Catalog #A110UK). FIGS. 8A and 8B show the ability to separate matriptase-activated (FIG. 8A) or MMP14-activated (FIG. 8B) conjugated activatable antibodies from intact conjugated activatable antibodies.

### Example 5. Evidence of Partial Response in Multiple Subjects Following Treatment with Anti-CD166 Activatable Antibody Drug Conjugate

This example demonstrates that administration of intact anti-CD166 activatable antibody conjugated to maytansinoid toxin DM4 through an SPDB linker (Combination 55) results in anti-tumor activity, including unconfirmed partial responses in multiple treated subjects with a range of tumor types.

In one example, the subject presented with head and neck squamous cell carcinoma (HNSCC), exhibiting only target lesions and no non-target lesions at initial screening. The subject was not observed to develop any new tumors while on study. The subject was treated with 5 mg/kg of intact anti-CD166 activatable antibody conjugated to maytansinoid toxin DM4 through an SPDB linker (Combination 55) every three (3) weeks. The administered dosage of the conjugated activatable antibody was based on the subject's adjusted ideal body weight.

The subject experienced a −31.7% change in tumor burden from initial screening (41 mm) to Cycle 3 visit (28 mm) i.e. 9 weeks after first administration. At the Cycle 6 visit i.e. 18 weeks after first administration, the subject had a tumor burden of (31.6 mm). Thus, the subject experienced an unconfirmed Partial Response since initial screening based on the RECIST v1.1 classification.

As shown in Tables 8A and 8B, an exemplary study of multiple subjects treated with at least 4 mg/kg of the conjugated activatable antibody (Combination 55) at different cut-offs is summarized. The summarized assessments for evaluable subjects are based post-baseline response assessments based on the RECIST v1.1 classification. “Disease control” refers to the sum of subjects exhibited unconfirmed complete response (CR), unconfirmed partial response (PR), and stable disease. The stable disease classification included subjects that had at least one stable disease assessment ≥7 weeks after the treatment start date and exhibited neither complete response nor progressive disease following treatment. “Not evaluable” included patients with stable disease with only one evaluable post-baseline tumor scan <7 weeks from treatment start. “Early discontinuation” included patients who discontinued study without providing a post-baseline scan. Of the 7 patients in Table 8B who showed unconfirmed partial response, 5 patients experienced Grade ≥2 ocular toxicity which resulted in dose delay or discontinuation of study treatment.

TABLE 8A

| Dose | No. of Treated | Unconfirmed | Stable | Disease | Progressive | Not |
|---|---|---|---|---|---|---|
| (mg/kg) | Subjects (n) | Partial Response | Disease | Control | Disease | Evaluable |
| 4 | 7 | 0 | 4 | 4 | 3 | 0 |
| 5 | 7 | 2 | 2 | 4 | 3 | 0 |
| 6 | 7 | 0 | 2 | 2 | 2 | 3 |
| 7 | 5 | 0 | 2 | 2 | 3 | 0 |
| 8 | 8 | 1 | 2 | 3 | 4 | 1 |
| 9 | 2 | 2 | 0 | 2 | 0 | 0 |
| 10 | 1 | 0 | 0 | 0 | 1 | 0 |
| All | 45 | 5 | 12 | 17 | 16 | 4 |
| dosages | | | | | | |

TABLE 8B
(response-evaluable population with post-baseline disease assessment)

| Dose | No. of Treated | Unconfirmed | Stable | Progressive | Not | Early |
|---|---|---|---|---|---|---|
| (mg/kg) | Subjects (n) | Partial Response | Disease | Disease | Evaluable | Discont. |
| <4 | 10 | 0 | 0 | 7 (70%) | 1 (10%) | 2 (20%) |
| 4-5 | 19 | 3 (16%) | 5 (26%) | 7 (37%) | 0 | 4 (21%) |
| 6-7 | 18 | 0 | 5 (28%) | 6 (33%) | 3 (17%) | 4 (22%) |
| 8-9 | 20 | 3 (15%) | 7 (35%) | 7 (35%) | 0 | 3 (15%) |
| 10 | 4 | 1 (25%) | 0 | 3 (75%) | 0 | 0 |
| All | 71 | 7 (10%) | 17 (24%) | 30 (42%) | 4 (6%) | 13 (18%) |
| cohorts | | | | | | |

In five (5) exemplary subjects in the study, two (2) subjects presented with epithelial ovarian carcinoma, two (2) subjects presented with breast carcinoma, and one (1) subject presented with head and neck squamous cell carcinoma (HNSCC). The subjects were treated with intact anti-CD166 activatable antibody conjugated to maytansinoid toxin DM4 through an SPDB linker (Combination 55) every three (3) weeks. The administered dosage of the conjugated activatable antibody was based on the subject's adjusted ideal body weight. For each of these subjects, the amount of administered conjugated activatable antibody and the approximate decrease in tumor burden (i.e. tumor shrinkage) from their respective baseline measurement is summarized in Table 9.

TABLE 9

| | Dosage | Approx. | Weeks |
|---|---|---|---|
| Cancer | per Cycle | Change in | Post-Treatment |
| Type | (mg/kg) | Tumor Burden | Initiation |
| HNSCC | 5 | −30% | 8 |
| Ovarian | 5 | −30% | 16 |
| Breast | 8 | −50% | 7 |
| Ovarian | 9 | −35% | 6 |
| Breast | 9 | −85% | 5 |

In the subject presenting with triple-negative breast carcinoma and treated with 9 mg/kg of the conjugated activatable antibody, a transverse CT scan performed at baseline and 8 weeks after treatment initiation showed tumor shrinkage in the lung and lymph nodes consistent with an unconfirmed partial response in at least two cross-sections. This subject had previously relapsed following treatment with multiple lines of chemotherapy and localized radiotherapy.

In the pembrolizumab-refractory subject presenting with triple-negative breast carcinoma and treated with 8 mg/kg of the conjugated activatable antibody, a metastasis presenting as a skin lesion prior to treatment resolved over three cycles of treatment with the conjugated activatable antibody. This subject had previously relapsed following treatment with neo-adjuvant cytoreductive chemotherapy, surgery, and radiotherapy.

These exemplary results demonstrate that dosages ≥4 mg/kg of the DM4-conjugated anti-CD166 activatable antibody demonstrated anti-tumor activity, including unconfirmed partial responses in a range of tumors.

### Example 6. Pharmacokinetics of Total and Intact Anti-CD166 Activatable Antibodies and Metabolites in Human Subjects Following Treatment

This example demonstrates the pharmacokinetics of total and intact anti-CD166 activatable antibody conjugated to maytansinoid toxin DM4 through an SPDB linker (Combination 55) following administration to human subjects.

In the above-described dose-escalation segment of the trial, the study was designed to assess the pharmacokinetics (PK) and ADA in subjects receiving doses of 0.25 mg/kg to 4.0 mg/kg (based on the subject's adjusted ideal body weight) of the conjugated anti-CD166 activatable antibody (Combination 55). For the PK studies, multiple analyses were used to determine the serum levels of (1) intact activatable anti-CD166 antibody both with and without conjugated DM4, (2) total (i.e. both intact and cleaved) anti-CD166 activatable antibody both with and without conjugated DM4, (3) total (i.e. both intact and cleaved) anti-CD166 activatable antibody with conjugated DM4, (4) free DM4, and (5)S-methyl DM4, a cytotoxic DM4 metabolite.

The studies were performed by assaying blood samples drawn from human subjects receiving the intact conjugated anti-CD166 activatable antibody (Combination 55). In Cycle 1 (i.e. the administration of the 1^{st} round of drug), the study was designed such that blood samples are drawn from the assessed subjects pre-infusion, at the end of infusion, and on days 2, 3, 4, 8, and 15 during the subject's visit. In subsequent Cycles 2, 4, 6, 8, and every 8 Cycles thereafter, the study was designed such that blood samples are drawn pre-infusion for each Cycle. In Cycle 3, the study was designed such that blood samples are drawn pre-infusion, at the end of infusion, and on days 8 and 15 during the subject's visit. The study was designed to draw a final blood sample at the end of the trial during the subject's visit.

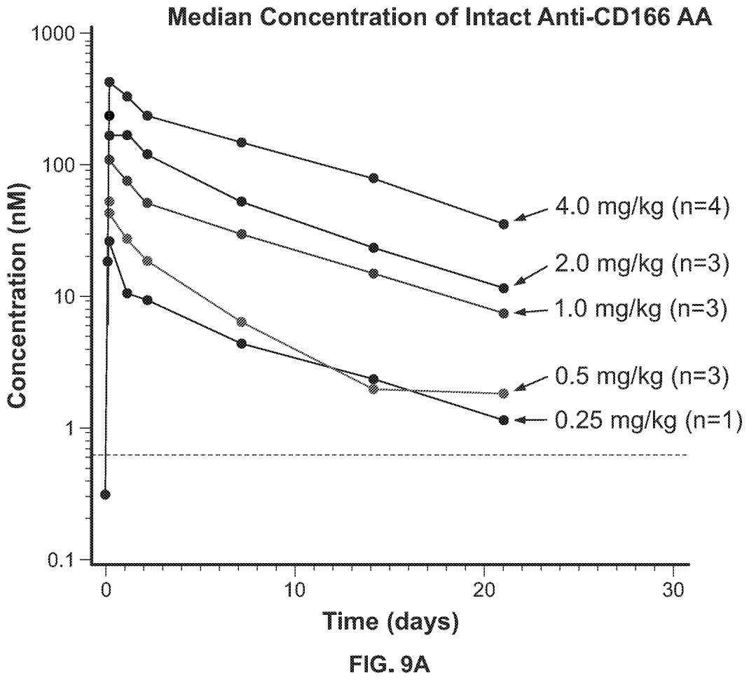

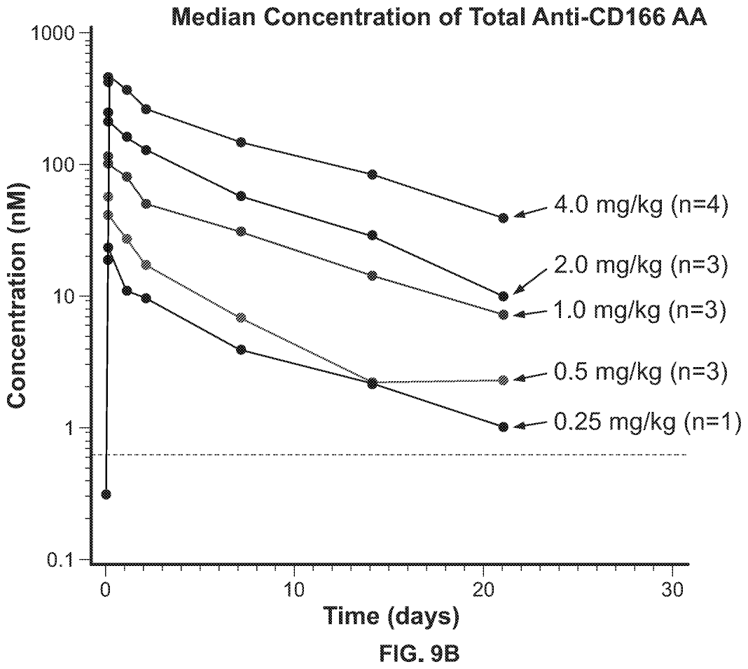

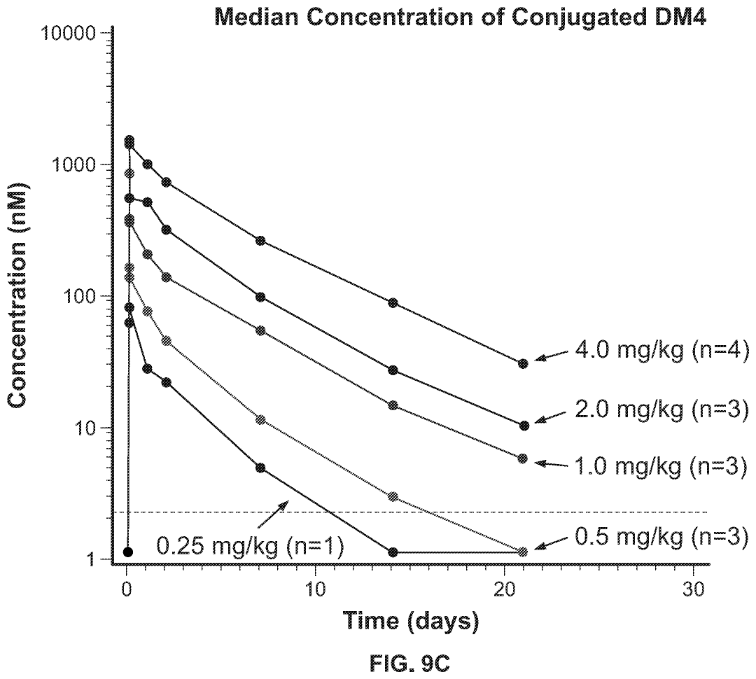

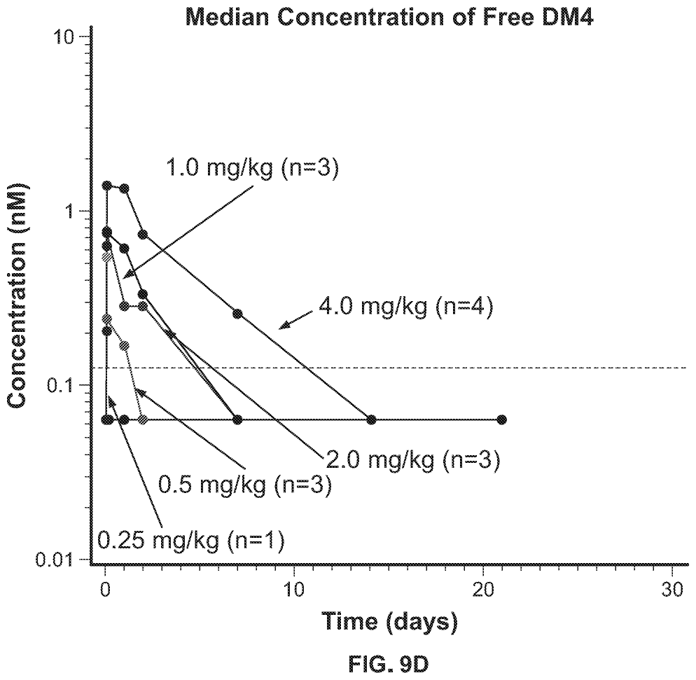

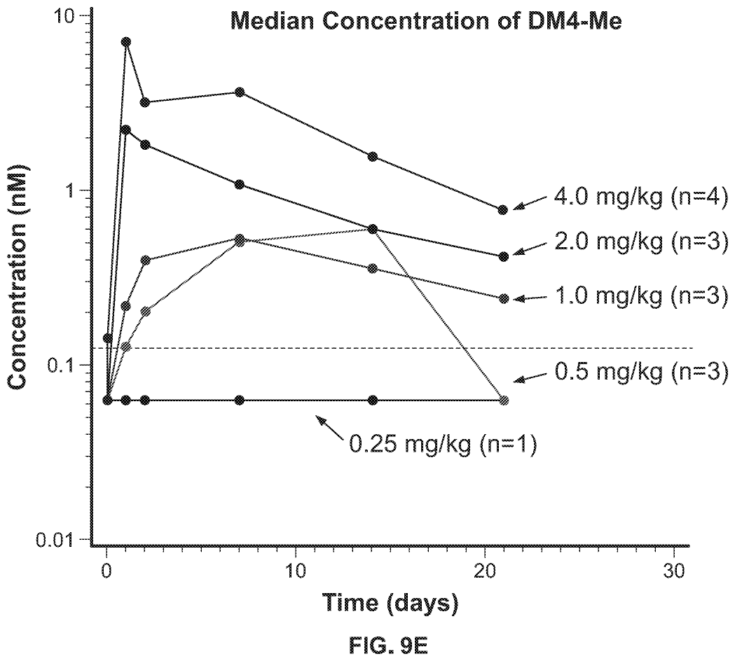

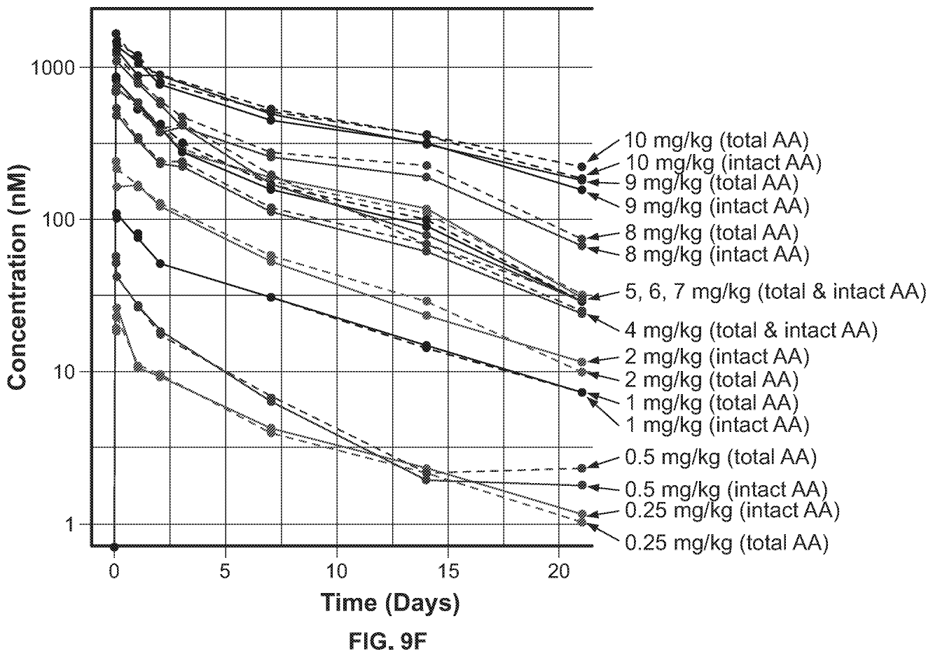

As shown in FIGS. 9A-9F, the exemplary results of the PK analysis following administration of the indicated dosages of Combination 55 are depicted. In each graph, the dotted line indicates the lower level of quantitation (LLOQ) for the respective assays, and points below this line are assigned a value of LLOQ/2. In FIG. 9A, the graph shows the serum concentrations over time of intact (i.e., uncleaved) anti-CD166 activatable antibody that are either unconjugated or conjugated to DM4 following administration of Combination 55 at the indicated dosage (based on AIBW) to human subjects. In FIG. 9B, the graph shows the serum concentrations over time of total (i.e., uncleaved and cleaved) anti-CD166 activatable antibody that is conjugated to DM4 following administration of Combination 55 at the indicated dosage (based on AIBW) to human subjects. In FIG. 9C, the graph shows the serum concentrations over time of free DM4 following administration of Combination 55 at the indicated dosage (based on AIBW) to human subjects. In FIG. 9D, the graph shows the serum concentrations over time of S-methyl DM4 (DM4-Me) following administration of Combination 55 at the indicated dosage (based on AIBW) to human subjects. In FIG. 9E, the graph shows the serum concentrations over time of total (i.e., uncleaved and cleaved) anti-CD166 activatable antibody that are either unconjugated or conjugated to DM4 following administration of Combination 55 at the indicated dosage (based on AIBW) to human subjects. In FIG. 9F, the graph shows the serum concentrations over time of total (i.e., uncleaved and cleaved) anti-CD166 activatable antibody that are either unconjugated or conjugated to DM4 following administration of Combination 55 at the indicated dosage (based on AIBW) to human subjects. The dotted lines indicate the amounts of total anti-CD166 activatable antibody (AA) and the solid lines indicate the amounts of intact anti-CD166 activatable antibody (AA).

In some exemplary studies of the present disclosure, conjugated activatable anti-CD166 antibody (Combination 55) was administered to human subjects and the amount the activatable anti-CD166 antibody within the subjects' tumors that was in activated (e.g., cleaved) form was determined. In these exemplary studies, the amount of cleaved activatable antibody was determined by Western blot analysis using monoclonal antibodies specific for the activatable antibody. In certain exemplary results of these studies, human subjects administered with dosages ranging from 4 to 8 mg/kg of the conjugated activatable antibody (based on AIBW) provided 11 samples of their tumor tissues, which were assayed for the concentration of activated activatable antibody. The exemplary results showed a relationship in which the amount of intratumoral activated activatable antibody increased with the initial dosage (ranging from 4 to 8 mg/kg).

The exemplary PK data shows that the anti-CD166 activatable antibody circulates in the serum predominantly in an intact form. Both free DM4 and DM4-Me circulated as <1.9 mol % of total anti-CD166 activatable antibody. Median intact anti-CD166 activatable antibody t_{1/2} ranged from 3.71 to 8.57 days. Upon multiple dosing, the accumulation ratio of minimum plasma concentration (Cₘᵢₙ) (Dose 3:Dose 1) for intact anti-CD166 activatable antibody did not exceed 1.34 and did not trend with dose.

The exemplary data also show that the ratio of intact to total anti-CD166 activatable antibody for Dose 1 AUC₀₋ₜₐᵤ (area under the curve evaluated until end of dosing interval) and Cₘₐₓ (maximum plasma concentration) appeared approximately consistent. Intact and total anti-CD166 activatable antibody exposure following a single dose of conjugated anti-CD166 activatable antibody generally increased with increasing dose as measured by AUC₀₋ₜₐᵤ and Cₘₐₓ.

### Example 7. Determination of Maximum Tolerated Dose (MTD) in Subjects Following Treatment with Anti-CD166 Activatable Antibody

This example demonstrates that administration of intact anti-CD166 activatable antibody conjugated to maytansinoid toxin DM4 through an SPDB linker (Combination 55) up to 10 mg/kg (based on adjusted ideal body weight) did not result in reaching a maximum tolerated dose (MTD).

In this example, designated as Part A2 of the study, subjects presented with breast carcinoma, non-small cell lung carcinoma (NSCLC), epithelial ovarian carcinoma, endometrial carcinoma, or head and neck squamous cell carcinoma (HNSCC). In addition, the subjects showed high expression of CD166 in their tumors, which was defined as immunohistochemistry (IHC) staining of ≥50% of tumor cells staining at 3+ (strong) intensity in an archival tumor tissue sample, where only membrane-associated staining within tumor cells were evaluated for these criteria. The subjects were administered with 4 mg/kg, 5 mg/kg, 6 mg/kg, 7 mg/kg, 8 mg/kg, 9 mg/kg, or 10 mg/kg every three (3) weeks in a dose escalation study of intact anti-CD166 activatable antibody conjugated to maytansinoid toxin DM4 through an SPDB linker (Combination 55). The administered dosage of the conjugated activatable antibody was based on the subject's adjusted ideal body weight. Assessment for dose-limiting toxicity (DLT) for each subject was performed during the 21 days after administration.

During the DLT assessment period, adverse events were captured according to the NCI Common Terminology Criteria for Adverse Events (NCI CTCAE) v4.03. Based on these criteria for adverse events (AEs), DLTs were defined as treatment-related Grade 5 AEs, certain treatment-related Grade 4 AEs (including Grade 4 ocular disorders), and certain treatment-related Grade 3 AEs. Based on these DLT criteria, an MTD would be determined as the dose above which ≥2 DLTs per 3 subjects in a cohort (or ≥2 DLTs per 6 subjects in a cohort) was observed during the assessment period. In this example, the MTD was not reached with dosages up to and including 10 mg/kg.

### Example 8. Observed Adverse Events in Subjects Following Monotherapy Treatment with Anti-CD166 Activatable Antibody Drug Conjugate

This example shows the amount and grade of adverse events observed in subjects following administration of intact anti-CD166 activatable antibody conjugated to maytansinoid toxin DM4 through an SPDB linker (Combination 55) up to 10 mg/kg (all dosages based on adjusted ideal body weight).

In this exemplary study, observations of adverse events were made in treated subjects from both Part A of the exemplary study (i.e. monotherapy dose escalation studies), in which subjects were treated with 0.25 mg/kg to 10 mg/kg of Combination 55, and Part A2 of the exemplary study (i.e., CD166+++ patients receiving monotherapy dosages of 4 mg/kg and above that were cleared in Part A), in which subjects were treated with 4 mg/kg to 10 mg/kg of Combination 55. All administered dosages were based on the subject's adjusted ideal body weight. Assessment for adverse events for each subject was performed during the 21 days after administration.

During the assessment period, adverse events were captured according to the NCI Common Terminology Criteria for Adverse Events (NCI CTCAE) v4.03. The number of treated subjects with the indicated type and grade of adverse events are shown in Table 10.

TABLE 10

| | | Subjects with | | | | | |
|---|---|---|---|---|---|---|---|
| | No. of | Treatment-Related | | Subjects with | | Subjects with | |
| | Treated | Adverse Events | | Ocular Toxicities | | Infusion Reactions | |
| Dose | Subjects | Grade | Grade | Grade | Grade | Grade | Grade |
| (mg/kg) | (n) | 1-2 | 3-4 | 1-2 | 3-4 | 1-2 | 3-4 |
| 0.25 | 1 | 0 | 0 | 0 | 0 | 0 | 0 |
| 0.5 | 3 | 1 | 0 | 0 | 0 | 0 | 0 |
| 1 | 3 | 2 | 0 | 0 | 0 | 1 | 0 |
| 2 | 3 | 3 | 0 | 0 | 0 | 1 | 0 |
| 4 | 10 | 7 | 1 | 2 | 0 | 1 | 0 |
| 5 | 9 | 6 | 3 | 5 | 1 | 2 | 0 |
| 6 | 9 | 7 | 2 | 2 | 0 | 3 | 0 |
| 7 | 9 | 6 | 2 | 3 | 0 | 3 | 0 |
| 8 | 12 | 6 | 5 | 6 | 2 | 2 | 1 |
| 9 | 7 | 3 | 2 | 1 | 2 | 0 | 0 |
| 10 | 5 | 1 | 2 | 1 | 1 | 0 | 0 |
| All | 71 | 42 (59%) | 17 (24%) | 20 (28%) | 6 (8%) | 13 (18%) | 1 (1%) |
| dosages | | | | | | | |

These exemplary data show that the MTD for the administered drug was not reached with dosages up to and including 10 mg/kg. In addition, these exemplary data also show that the administered drug showed a favorable safety profile in this exemplary study based on the number of subjects in which Grade 3-4 treatment-related adverse events (e.g., nausea, vomiting, fatigue, keratitis, hypokalemia, hyponatremia, peripheral neuropathy, liver function tests, and anemia) were observed. In this exemplary study, serious AEs (Grades 3-4) were observed in 27 (35%) patients. Examples of serious AEs occurring in ≥2 patients included nausea (n=4), vomiting (n=4), abdominal pain (n=3), small intestinal obstruction (n=3), hypokalemia (n=2), hyponatremia (n=2), infusion-related reaction (n=2), and pericardial effusion (n=2). In this exemplary study, treatment-related AEs (TRAEs) were observed in 69 (89%) patients; most were CTCAE grades 1 and 2. The most common (>10%) TRAEs of any grade were nausea (32%), fatigue (24%), decreased appetite (23%), diarrhea (19%), keratitis (19%), infusion-related reaction (18%), blurred vision (17%), vomiting (15%), and increased aspartate aminotransferase (13%) All events were medically manageable, with improvement or resolution following dose delay, discontinuation, and/or dose reduction. In this exemplary study, 18 patients had at least 1 treatment delay; the most common reasons for treatment delays included ocular toxicity (n=12, 67%) and peripheral neuropathy (n=4, 22%).

A summary of the number and percentage of subjects observed with Grade 3-4 treatment-related adverse events (TRAE) is summarized in Tables 11A and 11B.

TABLE 11A

| | No. of Subjects (≥2) Observed with Grade 3-4 TRAEs (% of Subjects Observed) | | | | | |
|---|---|---|---|---|---|---|
| | Total | | Metabolism | Liver | Gastro- | Nervous |
| Dose | Grade 3-4 | Eye | & Nutrition | Function | intestinal | System |
| (mg/kg) | TRAEs | Disorders | Disorders | Tests¹ | Disorders | Disorders |
| <4 | 0 | 0 | 0 | 0 | 0 | 0 |
| (n = 10) | | | | | | |
| 4-5 | 4 | 1 | 0 | 0 | 0 | 1 |
| (n = 19) | (21.1%) | (5.3%) | | | | (5.3%) |
| 6-7 | 4 | 0 | 2 | 0 | 1 | 2 |
| (n = 18) | (22.2%) | | (11.1%) | | (5.6%) | (11.1%) |
| 8-9 | 11 | 5 | 2 | 1 | 2 | 0 |
| (n = 23) | (52.4%) | (23.8%) | (9.5%) | (4.8%) | (9.5%) | |
| 10 | 4 | 1 | 0 | 3 | 1 | 0 |
| (n = 8) | (50%) | (12.5%) | | (37.5%) | (12.5%) | |

TABLE 11B

| | No. of Subjects (≥2) Observed with Grade 3-4 TRAEs (% of Subjects Observed) | | | | | | | | |
|---|---|---|---|---|---|---|---|---|---|
| Dose | | Increased | Increased | | | | | | |
| (mg/kg) | Keratitis | AST | ALT | Nausea | HN | Anemia | Fatigue | PSN | Vomiting |
| <4 | 0 | 0 | 0 | 0 | 0 | 0 | 0 | 0 | 0 |
| (n = 10) | | | | | | | | | |
| 4-5 | 1 | 0 | 0 | 0 | 0 | 1 | 1 | 1 | 0 |
| (n = 19) | (5%) | | | | | (5%) | (5%) | (5%) | |
| 6-7 | 0 | 0 | 0 | 1 | 2 | 1 | 0 | 1 | 1 |
| (n = 18) | | | | (6%) | (11%) | (6%) | | (6%) | (6%) |
| 8-9 | 4 | 1 | 1 | 2 | 1 | 0 | 0 | 0 | 1 |
| (n = 23) | (17%) | (4%) | (4%) | (9%) | (4%) | | | | (4%) |
| 10 | 1 | 3 | 2 | 1 | 0 | 0 | 1 | 0 | 0 |
| (n = 8) | (13%) | (38%) | (25%) | (13%) | | | (13%) | | |
| All | 6 | 4 | 3 | 4 | 3 | 2 | 2 | 2 | 2 |
| Cohorts | (8%) | (5%) | (4%) | (5%) | (4%) | (3%) | (3%) | (3%) | (3%) |
| (n = 78) | | | | | | | | | |

AST, aspartate aminotransferase;
ALT, alanine aminotransferase;
HN, hyponatremia;
PSN, Peripheral sensory neuropathy

At a given cut-off in this exemplary study, 9/78 patients (12%) had treatment-related adverse events (TRAE) that led to treatment discontinuation. Of these, keratitis was the TRAE that resulted in discontinuation in 6 patients, and blurred vision, peripheral neuropathy, and nausea was the TRAE that resulted in discontinuation in 1 patient each. Of these, 2 patients (2.6%) had Grade 4 TRAEs (1 each: keratitis, gamma-glutamyl transferase increased). In this study, ocular prophylaxis (e.g. steroidal eye drops) was introduced for the top 2 dose levels (e.g. 9 and 10 mg/kg).

### Example 9. Observed Activity in Multiple Subjects Following Treatment with Anti-CD166 Activatable Antibody Drug Conjugate

This exemplary study provides exemplary results demonstrating that administration of intact anti-CD166 activatable antibody conjugated to maytansinoid toxin DM4 through an SPDB linker (Combination 55) results in anti-tumor activity in certain subjects, including unconfirmed partial responses in multiple treated subjects with a range of tumor types.

In accordance with Part A or Part A2 of the exemplary study described herein, anti-CD166 activatable antibody conjugated to maytansinoid toxin DM4 through an SPDB linker (Combination 55) was administered to human subjects at various dosages every three (3) weeks. The average size of the subject's tumor lesions was measured prior to and after administration.

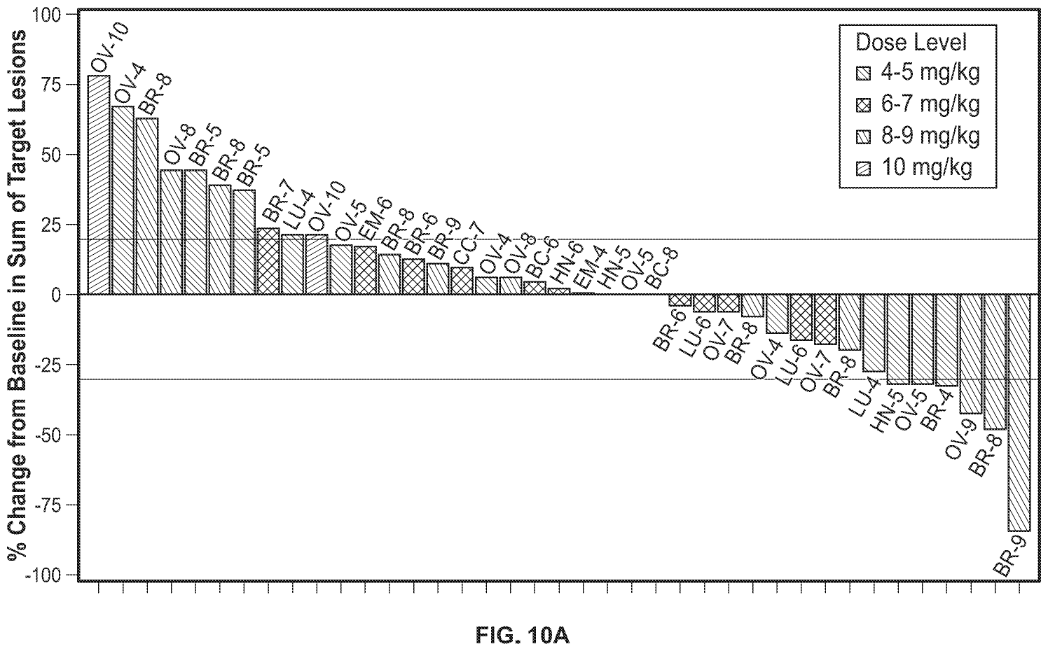

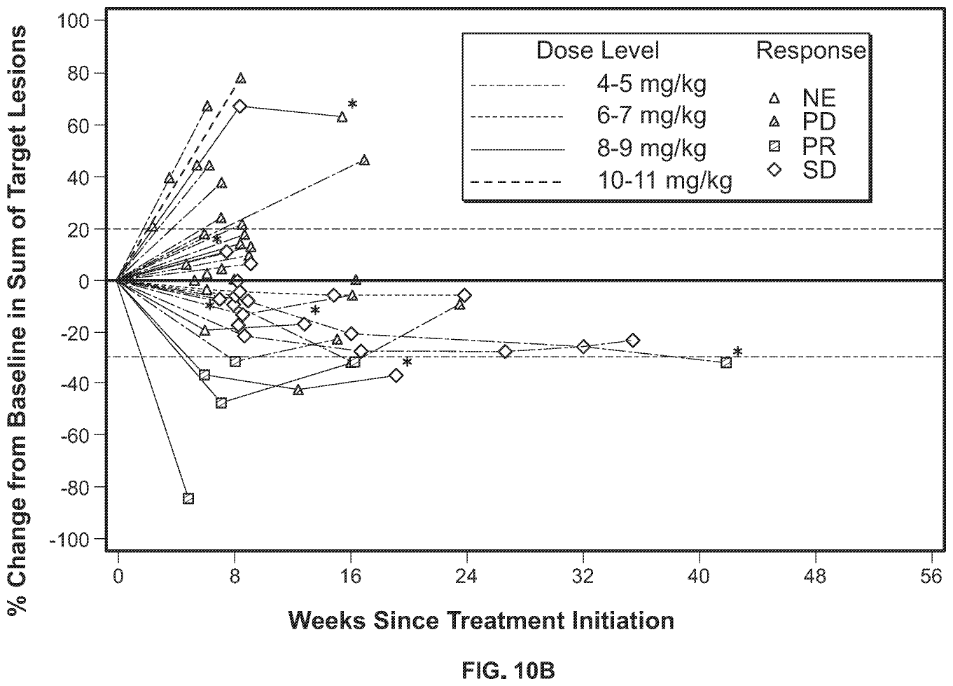

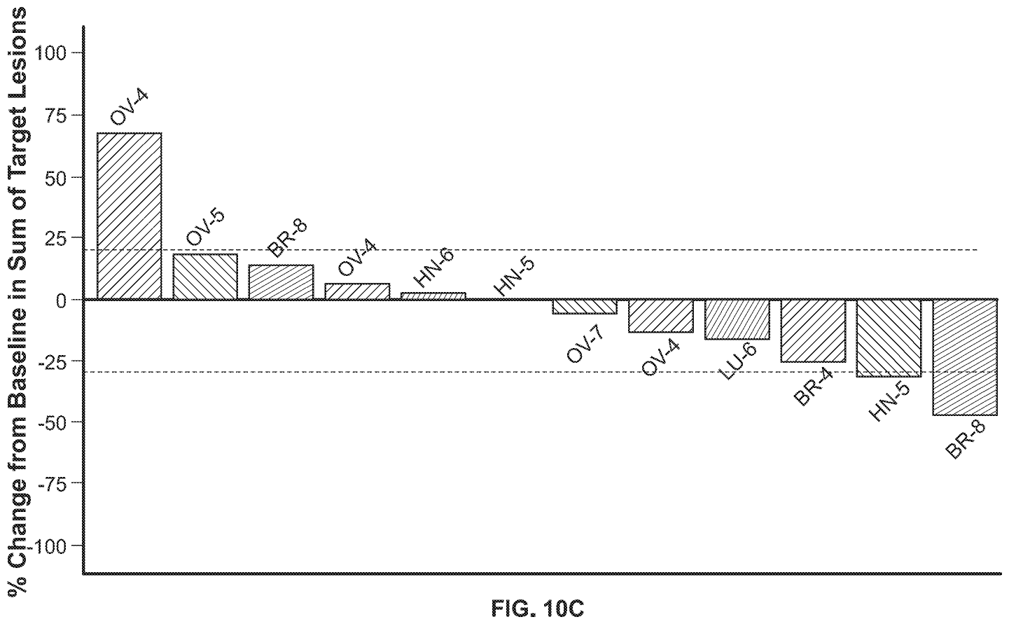

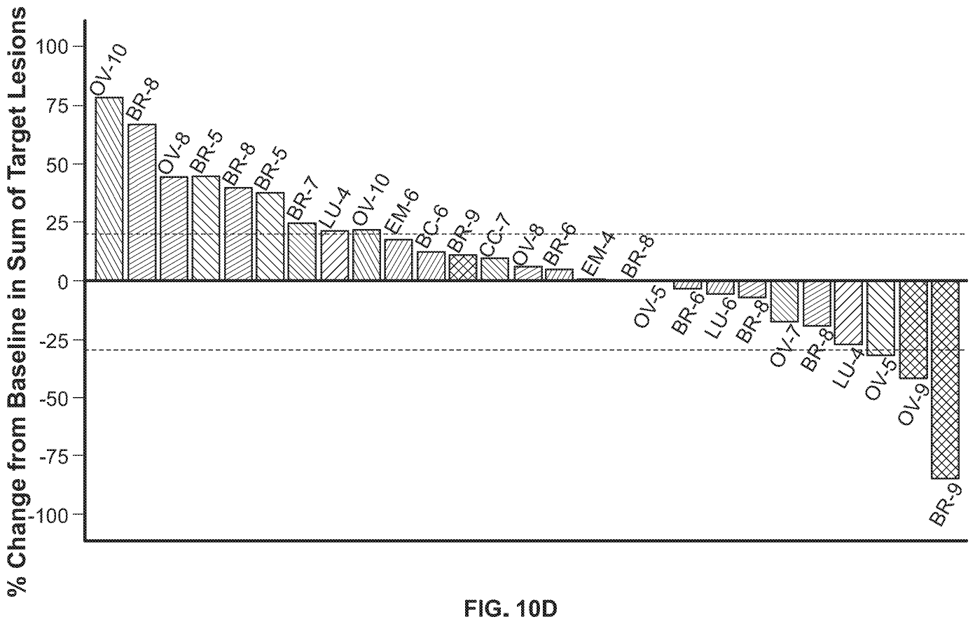

Referring to FIGS. 10A-10C, the subjects presented with the indicated cancer (BR=breast carcinoma, CC=cholangiocarcinoma, EM=endometrial carcinoma, HN=head and neck squamous cell carcinoma, LU=non-small cell lung carcinoma, OV=epithelial ovarian carcinoma) and were administered with the conjugated activatable anti-CD166 antibody (Combination 55) at a dosage of the associated number in mg/kg based on adjusted ideal body weight (e.g., OV-10 corresponds to the subject presenting with epithelial ovarian cancer and was administered with a dosage of 10 mg/kg AIBW of the drug every three (3) weeks). Referring to FIG. 10B, a plot showing the percent change in tumor burden for multiple patients with various indications at the indicated dosage in this exemplary study. FIG. 10B shows patients that showed Partial Response (PR), Progressive Disease (PD), Stable Disease (SD), or Not Evaluable (NE). These figures do not include patients who were evaluable for efficacy but have (1) incomplete scan data at the time of the cut-off (n=9), or (2) non-measurable disease at baseline (n=2). Patients (n=3) with one evaluable post-baseline tumor scan <7 weeks from treatment start assessed as Stable Disease were considered to have a best overall response of Not Evaluable. As shown in the exemplary results of FIG. 10A, the graph shows the best percentage change of the sum of each subject's target lesion measurements from their respective baseline measurements. These exemplary results demonstrate that dosages ≥4 mg/kg of the DM4-conjugated anti-CD166 activatable antibody demonstrated anti-tumor activity based on tumor shrinkage in multiple cancer indications.

As shown in the exemplary results shown in FIGS. 10C and 10D, the graphs show the best percentage change of the sum of each subject's target lesion measurements from their respective baseline measurements. The subjects represented in FIG. 10C were previously treated with PD-pathway inhibitors, while the subjects represented in FIG. 10D were not previously treated with PD-pathway inhibitors. These exemplary results demonstrate that dosages ≥4 mg/kg of the DM4-conjugated anti-CD166 activatable antibody demonstrated anti-tumor activity based on tumor shrinkage in multiple cancer indications regardless of whether the subject had been previously treated with PD-pathway inhibitors.

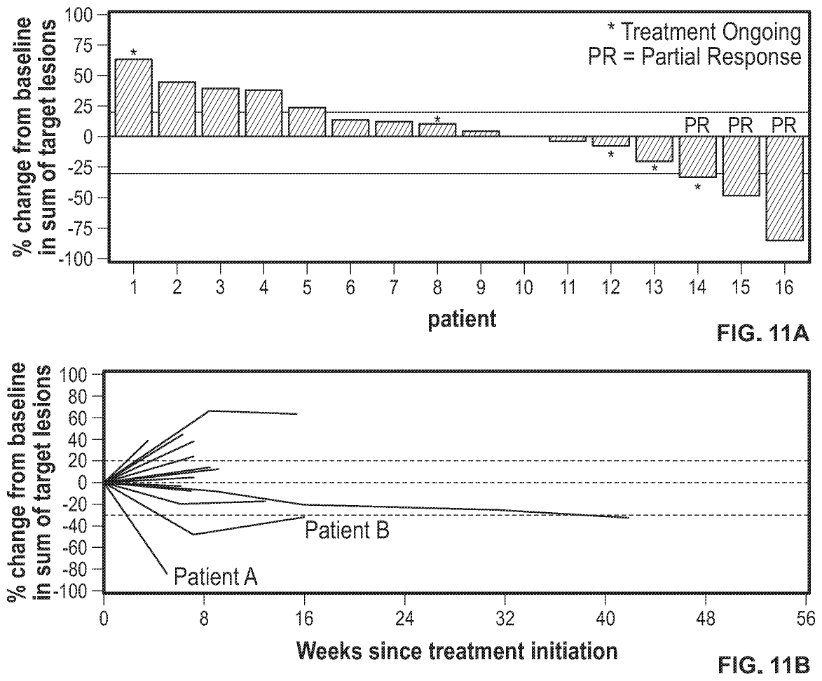

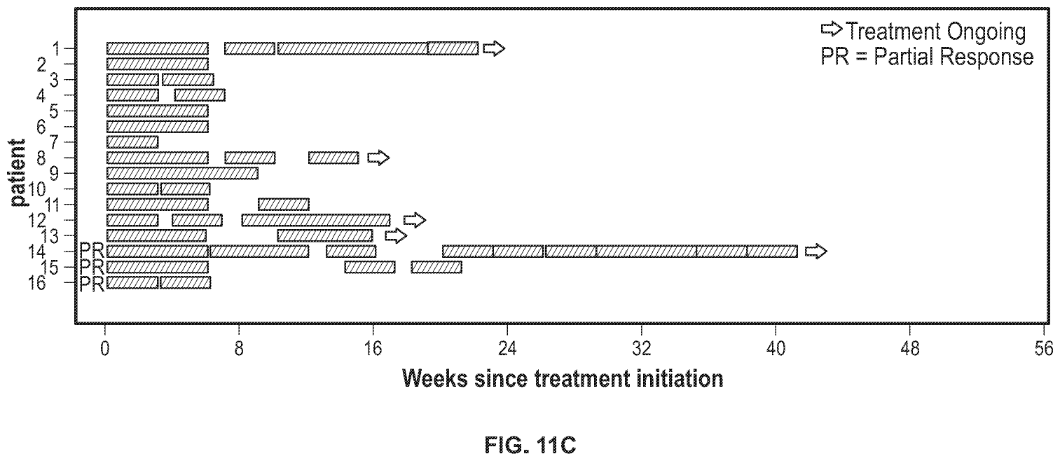

Referring to FIGS. 11A and 11B, these exemplary results show the response of patients with breast cancer that were treated with 4-10 mg/kg of the anti-CD166 activatable antibody (Combination 55) conjugated to DM4 in this exemplary study. These graphs show the best percentage change of the sum of each subject's target lesion measurements from their respective baseline measurements. FIG. 11C shows the times of treatment for the corresponding patients, where each bar indicates a 3-week period following administration of the drug, and gaps indicating time periods in which the patient did not receive a subsequent administration after 3 weeks following the previous administration. Patient 1 had a follow-up tumor scan with incomplete efficacy assessment and shows as Not Evaluable in plot for this assessment. Patients (N=3) who were evaluable for efficacy but have incomplete scan data entered as of the data cut-off date are not included in the figure. Patients (N=2) with 1 evaluable post-baseline tumor scan <7 weeks from treatment start assessed as stable disease will be considered to have best overall response of not evaluable. These exemplary results demonstrate that 3 of these response-evaluable patients that were administered dosages ≥4 mg/kg of the DM4-conjugated anti-CD166 activatable antibody showed Partial Response at the cut-off date.

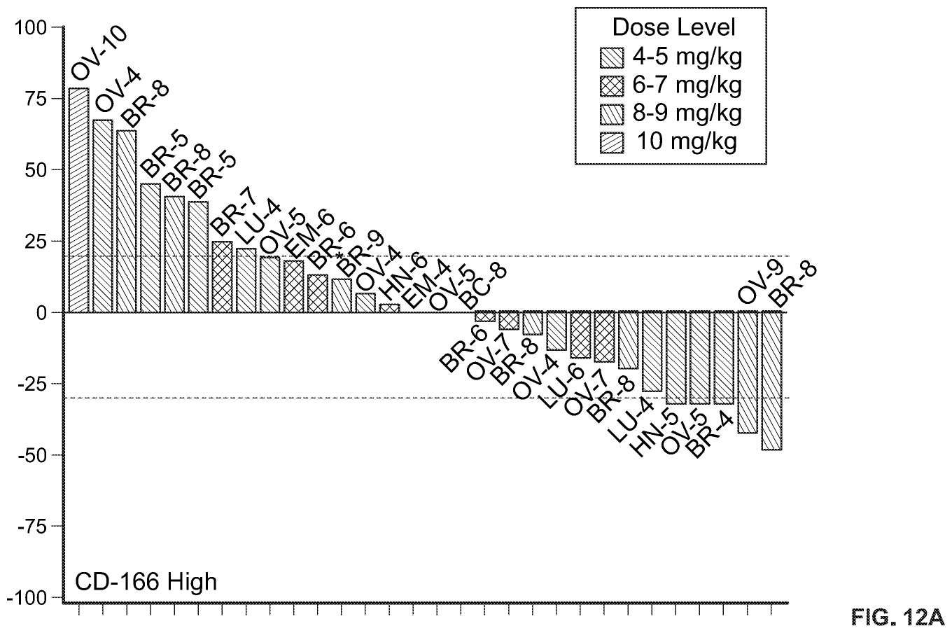

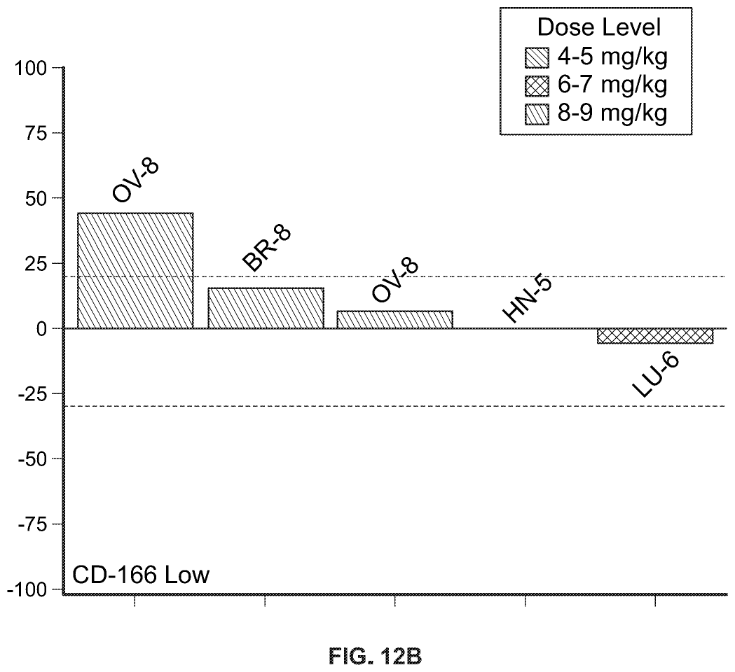

As shown in the exemplary results in FIGS. 12A and 12B, these exemplary results show the response of patients in Part A and/or A2 and their level of expression of CD166 in their tumor cells. As described herein, in this exemplary study the patients were treated with 4-10 mg/kg of the anti-CD166 activatable antibody (Combination 55) conjugated to DM4. FIG. 12A shows the results of those patients with high CD166 expression, which was defined as immunohistochemistry (IHC) staining of ≥50% of tumor cells staining at 3+ (strong) intensity in an archival tumor tissue sample, where only membrane-associated staining within tumor cells were evaluated for these criteria. FIG. 12B shows the results of those patients with lower CD166 expression. These graphs show the best percentage change of the sum of each subject's target lesion measurements from their respective baseline measurements. These exemplary results demonstrate a correlation between the level of CD166 expression in the patient with the efficacy of the treatment with the drug, including the observation that patients having high CD166 expression showed Partial Response.

### Illustrative Embodiments

The invention may be defined by reference to the following illustrative enumerated embodiments.

Embodiment 1. A method of treating, alleviating a symptom of, or delaying the progression of a cancer in a subject, the method comprising administering a therapeutically effective amount of an activatable antibody (AA) conjugated to an agent to a subject in need thereof, wherein the subject is administered the AA conjugated to an agent at a dose of greater than 6 mg/kg to about 10 mg/kg,

(A) wherein the AA comprises:
a. an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 240;
b. a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and
c. a cleavable moiety (CM) coupled to the AB, wherein the CM is a polypeptide that functions as a substrate for a protease, and wherein the CM comprises the amino acid sequence of SEQ ID NO: 76; or

(B) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 314; or

(C) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 246;
   and/or, or stated in an alternative manner, Embodiment 1 is an activatable antibody (AA) conjugated to an agent for use in treating, alleviating a symptom of, or delaying the progression of a cancer in a subject,

(A) wherein the AA comprises:
a. an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 240;
b. a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and
c. a cleavable moiety (CM) coupled to the AB, wherein the CM is a polypeptide that functions as a substrate for a protease, and wherein the CM comprises the amino acid sequence of SEQ ID NO: 76; or

(B) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 314; or

(C) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 246;

Embodiment 2. The method or use of embodiment 1, wherein the cancer is breast carcinoma, castration-resistant prostate carcinoma, cholangiocarcinoma, endometrial carcinoma, epithelial ovarian carcinoma, head and neck squamous cell carcinoma, or non-small cell lung cancer.

Embodiment 3. The method or use of embodiment 1, wherein the cancer is breast carcinoma, prostate carcinoma, cholangiocarcinoma, endometrial carcinoma, ovarian carcinoma, head and neck carcinoma, or lung cancer.

Embodiment 4. A method of inhibiting or reducing the growth, proliferation, or metastasis of cells expressing CD166 in a subject, comprising administering a therapeutically effective amount of an activatable antibody (AA) conjugated to an agent to a subject in need thereof, wherein the subject is administered the AA conjugated to an agent at a dose of greater than 6 mg/kg to about 10 mg/kg, wherein the AA comprises:

a. an antibody or an antigen binding fragment thereof (AB) that specifically binds to mammalian CD166, wherein the AB comprises a heavy chain comprising an amino acid sequence of SEQ ID NO: 480, and a light chain comprising an amino acid sequence of SEQ ID NO: 240;

b. a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and

c. a cleavable moiety (CM) coupled to the AB, wherein the CM is a polypeptide that functions as a substrate for a protease, and wherein the CM comprises the amino acid sequence of SEQ ID NO: 76.

and/or, or stated in an alternative manner Embodiment 3 is an activatable antibody (AA) conjugated to an agent for use in inhibiting or reducing the growth, proliferation, or metastasis of cells expressing CD166, for example for the treatment of cancer in a subject, wherein the AA comprises:

a. an antibody or an antigen binding fragment thereof (AB) that specifically binds to mammalian CD166, wherein the AB comprises a heavy chain comprising an amino acid sequence of SEQ ID NO: 480, and a light chain comprising an amino acid sequence of SEQ ID NO: 240;

b. a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and

c. a cleavable moiety (CM) coupled to the AB, wherein the CM is a polypeptide that functions as a substrate for a protease, and wherein the CM comprises the amino acid sequence of SEQ ID NO: 76, and

wherein the AA is for administration in a therapeutically effective amount to a subject in need thereof.

Embodiment 5. The method or use of embodiment 4, wherein the subject suffers from breast carcinoma, castration-resistant prostate carcinoma, cholangiocarcinoma, endometrial carcinoma, epithelial ovarian carcinoma, head and neck squamous cell carcinoma, or non-small cell lung cancer.

Embodiment 6. The method or use of embodiment 4, wherein the subject suffers from breast carcinoma, prostate carcinoma, cholangiocarcinoma, endometrial carcinoma, ovarian carcinoma, head and neck carcinoma, or lung cancer.

Embodiment 7. The method of embodiment 4, wherein the cells are breast cells, prostate cells, endometrial cells, ovarian cells, head or neck cells, bile duct cells, or lung cells.

Embodiment 8. The method of any one of embodiments 1-7, wherein the agent is a maytansinoid or derivative thereof.

Embodiment 9. The method of any one of embodiments 1-8, wherein the agent is DM4.

Embodiment 10. The method of any one of embodiments 1-9, wherein the DM4 is conjugated to the AA via a linker.

Embodiment 11. The method or use of embodiment 10, wherein the linker comprises an SPBD moiety.

Embodiment 12. The method or use of any one of embodiments 1-11, wherein the AB is linked to the CM.

Embodiment 13. The method or use of any one of embodiments 1-12, wherein the MM is linked to the CM such that the AA in an uncleaved state comprises the structural arrangement from N-terminus to C-terminus as follows: MM-CM-AB or AB-CM-MM.

Embodiment 14. The method or use of any one of embodiments 1-13, wherein the AA comprises a linking peptide between the MM and the CM.

Embodiment 15. The method or use of any one of embodiments 1-14, wherein the AA comprises a linking peptide between the CM and AB.

Embodiment 16. The method or use of embodiment 14, wherein linking peptide comprises the amino acid sequence of SEQ ID NO: 479.

Embodiment 17. The method or use of any one of embodiments 1-16, wherein the AA comprises a linking peptide between the CM and the AB.

Embodiment 18. The method or use of embodiment 17, wherein linking peptide comprises the amino acid sequence of GGS.

Embodiment 19. The method or use of any one of embodiments 1-18, wherein the AA comprises a first linking peptide (LP1) and a second linking peptide (LP2), and wherein the AA in the uncleaved state has the structural arrangement from N-terminus to C-terminus as follows: MM-LP1-CM-LP2-AB or AB-LP2-CM-LP1-MM.

Embodiment 20. The method or use of any one of embodiments 1-19, wherein the light chain is linked to a spacer at its N-terminus.

Embodiment 21. The method or use of embodiment 20, wherein the spacer comprises the amino acid sequence of SEQ ID NO: 305.

Embodiment 22. The method or use of any one of embodiments 1-21, wherein the MM and CM are linked to the light chain.

Embodiment 23. The method or use of embodiment 22, wherein the MM is linked to the CM such that the AA in an uncleaved state comprises the structural arrangement from N-terminus to C-terminus on its light chain as follows: spacer-MM-LP1-CM-LP2-light chain.

Embodiment 24. The method or use of embodiment 23, wherein the spacer comprises the amino acid sequence of SEQ ID NO: 305, LP1 comprises the amino acid sequence of SEQ ID NO: 479, and LP2 comprises the amino acid sequence of GGS.

Embodiment 25. The method or use of any one of embodiments 1-24, wherein the light chain of the AA comprises the sequence of SEQ ID NO: 314.

Embodiment 26. The method or use of any one of embodiments 1-25, wherein the light chain of the AA comprises the sequence of SEQ ID NO: 246.

Embodiment 27. The method or use of any one of embodiments 1-26, wherein the subject is at least 18 years of age

Embodiment 28. The method or use of any one of embodiments 1-27, wherein the subject has an ECOG performance status of 0-1.

Embodiment 29. The method or use of any one of embodiments 1-28, wherein the subject has a histologically confirmed diagnosis of an active metastatic cancer

Embodiment 30. The method or use of any one of embodiments 1-28, wherein the subject has a histologically confirmed diagnosis of a locally advanced unresectable solid tumor

Embodiment 31. The method or use of any one of embodiments 1-30, wherein the subject has a life expectancy of at least 3 months at the time of administration or use.

Embodiment 32. The method or use of any one of embodiments 1-31, wherein the subject has a breast carcinoma.

Embodiment 33. The method or use of embodiment 32, wherein the breast carcinoma is ER+.

Embodiment 34. The method or use of any one of embodiments 31-33 and has received prior anti-hormonal therapy and experienced disease progression.

Embodiment 35. The method or use of embodiment 32, wherein the subject has a triple negative breast cancer and has undergone at least two prior lines of therapy.

Embodiment 36. The method or use of any one of embodiments 1-31, wherein the subject has castration-resistant prostate carcinoma.

Embodiment 37. The method or use of embodiment 36, wherein the subject has received at least one prior therapy.

Embodiment 38. The method or use of any one of embodiments 1-31, wherein the subject has cholangiocarcinoma.

Embodiment 39. The method or use of embodiment 38, wherein the subject has failed at least one prior line of gemcitabine-containing regimen.

Embodiment 40. The method or use of any one of embodiments 1-31, wherein the subject has endometrial carcinoma.

Embodiment 41. The method or use of embodiment 40, wherein the subject has received at least one platinum-containing regimen for extra-uterine or advanced disease.

Embodiment 42. The method or use of any one of embodiments 1-31, wherein the subject has epithelial ovarian carcinoma.

Embodiment 43. The method or use of embodiment 42, wherein the subject has a platinum-resistant carcinoma.

Embodiment 44. The method or use of embodiment 42, wherein the subject has a platinum refractory ovarian carcinoma.

Embodiment 45. The method or use of embodiment 42, wherein the subject has a BRCA mutation and is refractory to or otherwise ineligible for PARP inhibitors.

Embodiment 46. The method or use of embodiment 42, wherein the subject has a non-BRCA mutation.

Embodiment 47. The method or use of any one of embodiments 1-31, wherein the subject has head and neck small cell carcinoma (HNSCC).

Embodiment 48. The method or use of embodiment 47, wherein the subject has received at least one platinum-containing regimen.

Embodiment 49. The method or use of embodiment 47, wherein the subject has received at least one PD-1/PD-L1 inhibitor.

Embodiment 50. The method or use of any one of embodiments 1-31, wherein the subject has non-small cell lung cancer (NSCLC).

Embodiment 51. The method or use of embodiment 50, wherein the subject has received at least one platinum-containing regimen.

Embodiment 52. The method or use of embodiment 50, wherein the subject has received at least one checkpoint inhibitor.

Embodiment 53. The method or use of embodiment 50, wherein the subject has received at least one PD-1/PD-L1 inhibitor.

Embodiment 54. The method or use of any one of embodiments 1-53, wherein the dose is about 7 mg/kg.

Embodiment 55. The method or use of any one of embodiments 1-53, wherein the dose is about 8 mg/kg.

Embodiment 56. The method or use of any one of embodiments 1-53, wherein the dose is about 9 mg/kg.

Embodiment 57. The method or use of any one of embodiments 1-53, wherein the dose is about 10 mg/kg.

Embodiment 58. The method or use of any one of embodiments 1-53, wherein the dose is greater than 6 mg/kg to about 7 mg/kg.

Embodiment 59. The method or use of any one of embodiments 1-53, wherein the dose is about 7 mg/kg to about 8 mg/kg.

Embodiment 60. The method or use of any one of embodiments 1-53, wherein the dose is about 8 mg/kg to about 9 mg/kg.

Embodiment 61. The method or use of any one of embodiments 1-53, wherein the dose is about 9 mg/kg to about 10 mg/kg.

Embodiment 62. The method or use of any one of embodiments 1-53, wherein the dose is greater than 6 mg/kg to about 8 mg/kg.

Embodiment 63. The method or use of any one of embodiments 1-53, wherein the dose is about 7 mg/kg to about 9 mg/kg.

Embodiment 64. The method or use of any one of embodiments 1-53, wherein the dose is about 8 mg/kg to about 10 mg/kg.

Embodiment 65. The method or use of any one of embodiments 1-53, wherein the AA conjugated to an agent is at a fixed dose of greater than 240 mg to about 1000 mg.

Embodiment 66. The method or use of any one of embodiments 1-53, wherein the AA conjugated to an agent is at a fixed dose of greater than 240 mg to about 400 mg.

Embodiment 67. The method or use of any one of embodiments 1-53, wherein the AA conjugated to an agent is at a fixed dose of greater than 600 mg to about 1000 mg.

Embodiment 68. The method or use of any one of embodiments 1-53, wherein the AA conjugated to an agent is at a fixed dose of greater than 240 mg to greater than 600 mg.

Embodiment 69. The method or use of any one of embodiments 1-53, wherein the AA conjugated to an agent is at a fixed dose of about 280 mg to about 700 mg.

Embodiment 70. The method or use of any one of embodiments 1-53, wherein the AA conjugated to an agent is at a fixed dose of about 320 mg to about 800 mg.

Embodiment 71. The method or use of any one of embodiments 1-53, wherein the AA conjugated to an agent is at a fixed dose of about 360 mg to about 900 mg.

Embodiment 72. The method or use of any one of embodiments 1-53, wherein the AA conjugated to an agent is at a fixed dose of about 400 mg to about 1000 mg.

Embodiment 73. The method or use of any one of embodiments 1-53, wherein the AA conjugated to an agent is at a fixed dose of greater than 240 mg to about 280 mg.

Embodiment 74. The method or use of any one of embodiments 1-53, wherein the AA conjugated to an agent is at a fixed dose of about 280 mg to about 320 mg.

Embodiment 75. The method or use of any one of embodiments 1-53, wherein the AA conjugated to an agent is at a fixed dose of about 320 mg to about 360 mg.

Embodiment 76. The method or use of any one of embodiments 1-53, wherein the AA conjugated to an agent is at a fixed dose of about 360 mg to about 400 mg.

Embodiment 77. The method or use of any one of embodiments 1-53, wherein the AA conjugated to an agent is at a fixed dose of greater than 600 mg to about 700 mg.

Embodiment 78. The method or use of any one of embodiments 1-53, wherein the AA conjugated to an agent is at a fixed dose of about 700 mg to about 800 mg.

Embodiment 79. The method or use of any one of embodiments 1-53, wherein the AA conjugated to an agent is at a fixed dose of about 800 mg to about 900 mg.

Embodiment 80. The method or use of any one of embodiments 1-53, wherein the AA conjugated to an agent is at a fixed dose of about 900 mg to about 1000 mg.

Embodiment 81. The method or use of any one of embodiments 1-53, wherein the AA conjugated to an agent is at a fixed dose of greater than 240 mg to about 320 mg.

Embodiment 82. The method or use of any one of embodiments 1-53, wherein the AA conjugated to an agent is at a fixed dose of about 280 mg to about 360 mg.

Embodiment 83. The method or use of any one of embodiments 1-53, wherein the AA conjugated to an agent is at a fixed dose of about 320 mg to about 400 mg.

Embodiment 84. The method or use of any one of embodiments 1-53, wherein the AA conjugated to an agent is at a fixed dose of greater than 600 mg to about 800 mg.

Embodiment 85. The method or use of any one of embodiments 1-53, wherein the AA conjugated to an agent is at a fixed dose of about 700 mg to about 900 mg.

Embodiment 86. The method or use of any one of embodiments 1-53, wherein the AA conjugated to an agent is at a fixed dose of about 800 mg to about 1000 mg.

Embodiment 87. The method or use of any one of embodiments 1-86, wherein the subject is administered the AA conjugated to an agent intravenously, or the AA is formulated for intravenous use.

Embodiment 88. The method or use of any one of embodiments 1-83, wherein the subject is administered the AA conjugated to an agent intravenously every 21 days or formulated for use every 21 days.

Embodiment 89. The method or use of any one of embodiments 1-83, wherein the subject is administered the AA conjugated to an agent intravenously every 14 days or formulated for use every 14 days.

Embodiment 90. The method or use of any one of embodiments 54-64 and 87-89, wherein the AA is conjugated to an agent with a dosage based on the subject's actual body weight.

Embodiment 91. The method or use of any one of embodiments 54-64 and 87-89, wherein the AA is conjugated to an agent with a dosage based on the subject's adjusted ideal body weight.

Embodiment 92. The method or use of any one of embodiments 1-91, wherein the subject has not had a history of acute or chronic corneal disease.

Embodiment 93. The method or use of any one of embodiments 1-92, wherein the method comprises administering to the subject a prophylactic treatment to reduce or prevent ocular adverse events.

Embodiment 94. The method or use of embodiment 93, wherein the prophylactic treatment is administered daily.

Embodiment 95. The method or use of embodiments 93 or 94, wherein the prophylactic treatment is one or more treatments selected from the group consisting of: lubricating artificial tears, brimonidine tartrate ophthalmic solution, application of a cool compress for the eyes, and topical steroid drops.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following.

1. A method of treating, alleviating a symptom of, or delaying the progression of a cancer in a subject, the method comprising:
administering a therapeutically effective amount of an activatable antibody (AA) conjugated to an agent to a subject in need thereof,
wherein the subject is administered the AA conjugated to an agent at a dose of greater than 6 mg/kg to about 10 mg/kg, and
(A) wherein the AA comprises:
a. an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 240;
b. a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and
c. a cleavable moiety (CM) coupled to the AB, wherein the CM is a polypeptide that functions as a substrate for a protease, and wherein the CM comprises the amino acid sequence of SEQ ID NO: 76; or
(B) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 314; or
(C) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 246.

2. The method of claim 1, wherein the cancer is breast carcinoma, castration-resistant prostate carcinoma, cholangiocarcinoma, endometrial carcinoma, epithelial ovarian carcinoma, head and neck squamous cell carcinoma, or non-small cell lung cancer.

3. The method of claim 1, wherein the cancer is breast carcinoma, prostate carcinoma, cholangiocarcinoma, endometrial carcinoma, ovarian carcinoma, head and neck carcinoma, or lung cancer.

4. A method of inhibiting or reducing the growth, proliferation, or metastasis of cells expressing CD166 in a subject, comprising:
administering a therapeutically effective amount of an activatable antibody (AA) conjugated to an agent to a subject in need thereof,
wherein the subject is administered the AA conjugated to an agent at a dose of greater than 6 mg/kg to about 10 mg/kg, and
(A) wherein the AA comprises:
a. an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 240;
b. a masking moiety (MM) coupled to the AB, wherein the MM inhibits the binding of the AB to the mammalian CD166 when the AA is in an uncleaved state, wherein the MM comprises the amino acid sequence of SEQ ID NO: 222; and
c. a cleavable moiety (CM) coupled to the AB, wherein the CM is a polypeptide that functions as a substrate for a protease, and wherein the CM comprises the amino acid sequence of SEQ ID NO: 76; or
(B) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 314; or
(C) wherein the AA comprises:
an antibody or an antigen binding fragment thereof (AB) that specifically binds to mammalian CD166, wherein the AB comprises a heavy chain comprising an amino acid sequence of SEQ ID NO: 480 or SEQ ID NO: 239, and a light chain comprising an amino acid sequence of SEQ ID NO: 246.

5. The method of claim 4, wherein the subject suffers from breast carcinoma, castration-resistant prostate carcinoma, cholangiocarcinoma, endometrial carcinoma, epithelial ovarian carcinoma, head and neck squamous cell carcinoma, or non-small cell lung cancer.

6. The method of claim 4, wherein the subject suffers from breast carcinoma, prostate carcinoma, cholangiocarcinoma, endometrial carcinoma, ovarian carcinoma, head and neck carcinoma, or lung cancer.

7. The method of claim 4, wherein the cells are breast cells, prostate cells, endometrial cells, ovarian cells, head or neck cells, bile duct cells, or lung cells.

8. The method of any one of claims 1-7, wherein the agent is a maytansinoid or derivative thereof.

9. The method of any one of claims 1-8, wherein the agent is DM4.

10. The method of any one of claims 1-9, wherein the DM4 is conjugated to the AA via a linker.

11. The method of claim 10, wherein the linker comprises an SPBD moiety.

12. The method of any one of claims 1-11, wherein the AB is linked to the CM.

13. The method of any one of claims 1-12, wherein the MM is linked to the CM such that the AA in an uncleaved state comprises the structural arrangement from N-terminus to C-terminus as follows: MM-CM-AB or AB-CM-MM.

14. The method of any one of claims 1-13, wherein the AA comprises a linking peptide between the MM and the CM.

15. The method of any one of claims 1-14, wherein the AA comprises a linking peptide between the CM and AB.

16. The method of claim 14, wherein linking peptide comprises the amino acid sequence of SEQ ID NO: 479.

17. The method of any one of claims 1-16, wherein the AA comprises a linking peptide between the CM and the AB.

18. The method of claim 17, wherein linking peptide comprises the amino acid sequence of GGS.

19. The method of any one of claims 1-18, wherein the AA comprises a first linking peptide (LP1) and a second linking peptide (LP2), and wherein the AA in the uncleaved state has the structural arrangement from N-terminus to C-terminus as follows: MM-LP1-CM-LP2-AB or AB-LP2-CM-LP1-MM.

20. The method of any one of claims 1-19, wherein the light chain is linked to a spacer at its N-terminus.

21. The method of claim 20, wherein the spacer comprises the amino acid sequence of SEQ ID NO: 305.

22. The method of any one of claims 1-21, wherein the MM and CM are linked to the light chain.

23. The method of claim 22, wherein the MM is linked to the CM such that the AA in an uncleaved state comprises the structural arrangement from N-terminus to C-terminus on its light chain as follows: spacer-MM-LP1-CM-LP2-light chain.

24. The method of claim 23, wherein the spacer comprises the amino acid sequence of SEQ ID NO: 305, LP1 comprises the amino acid sequence of SEQ ID NO: 479, and LP2 comprises the amino acid sequence of GGS.

25. The method of any one of claims 1-24, wherein the light chain of the AA comprises the sequence of SEQ ID NO: 314.

26. The method of any one of claims 1-25, wherein the light chain of the AA comprises the sequence of SEQ ID NO: 246.

27. The method of any one of claims 1-26, wherein the subject is at least 18 years of age

28. The method of any one of claims 1-27, wherein the subject has an ECOG performance status of 0-1.

29. The method of any one of claims 1-28, wherein the subject has a histologically confirmed diagnosis of an active metastatic cancer.

30. The method of any one of claims 1-28, wherein the subject has a histologically confirmed diagnosis of a locally advanced unresectable solid tumor.

31. The method of any one of claims 1-30, wherein the subject has a life expectancy of at least 3 months at the time of administration.

32. The method of any one of claims 1-31, wherein the subject has a breast carcinoma.

33. The method of claim 32, wherein the breast carcinoma is ER+.

34. The method of any one of claims 32-33, and has received prior anti-hormonal therapy and experienced disease progression.

35. The method of claim 32, wherein the subject has a triple negative breast cancer and has undergone at least two prior lines of therapy.

36. The method of any one of claims 1-31, wherein the subject has castration-resistant prostate carcinoma.

37. The method of claim 36, wherein the subject has received at least one prior therapy.

38. The method of any one of claims 1-31, wherein the subject has cholangiocarcinoma.

39. The method of claim 38, wherein the subject has failed at least one prior line of gemcitabine-containing regimen.

40. The method of any one of claims 1-31, wherein the subject has endometrial carcinoma.

41. The method of claim 40, wherein the subject has received at least one platinum-containing regimen for extra-uterine or advanced disease.

42. The method of any one of claims 1-31, wherein the subject has epithelial ovarian carcinoma.

43. The method of claim 42, wherein the subject has a platinum-resistant carcinoma.

44. The method of claim 42, wherein the subject has a platinum refractory ovarian carcinoma.

45. The method of claim 42, wherein the subject has a BRCA mutation and is refractory to or otherwise ineligible for PARP inhibitors.

46. The method of claim 42, wherein the subject has a non-BRCA mutation.

47. The method of any one of claims 1-31, wherein the subject has head and neck small cell carcinoma (HNSCC).

48. The method of claim 47, wherein the subject has received at least one platinum-containing regimen.

49. The method of claim 47, wherein the subject has received at least one PD-1/PD-L1 inhibitor.

50. The method of any one of claims 1-31, wherein the subject has non-small cell lung cancer (NSCLC).

51. The method of claim 50, wherein the subject has received at least one platinum-containing regimen.

52. The method of claim 50, wherein the subject has received at least one checkpoint inhibitor.

53. The method of claim 50, wherein the subject has received at least one PD-1/PD-L1 inhibitor.

54. The method of any one of claims 1-53, wherein the dose is about 7 mg/kg.

55. The method of any one of claims 1-53, wherein the dose is about 8 mg/kg.

56. The method of any one of claims 1-53, wherein the dose is about 9 mg/kg.

57. The method of any one of claims 1-53, wherein the dose is about 10 mg/kg.

58. The method of any one of claims 1-53, wherein the dose is greater than 6 mg/kg to about 7 mg/kg.

59. The method of any one of claims 1-53, wherein the dose is about 7 mg/kg to about 8 mg/kg.

60. The method of any one of claims 1-53, wherein the dose is about 8 mg/kg to about 9 mg/kg.

61. The method of any one of claims 1-53, wherein the dose is about 9 mg/kg to about 10 mg/kg.

62. The method of any one of claims 1-53, wherein the dose is greater than 6 mg/kg to about 8 mg/kg.

63. The method of any one of claims 1-53, wherein the dose is about 7 mg/kg to about 9 mg/kg.

64. The method of any one of claims 1-53, wherein the dose is about 8 mg/kg to about 10 mg/kg.

65. The method of any one of claims 1-53, wherein the subject is administered the AA conjugated to an agent at a fixed dose of greater than 240 mg to about 1000 mg.

66. The method of any one of claims 1-53, wherein the subject is administered the AA conjugated to an agent at a fixed dose of greater than 240 mg to about 400 mg.

67. The method of any one of claims 1-53, wherein the subject is administered the AA conjugated to an agent at a fixed dose of greater than 600 mg to about 1000 mg.

68. The method of any one of claims 1-53, wherein the subject is administered the AA conjugated to an agent at a fixed dose of greater than 240 mg to greater than 600 mg.

69. The method of any one of claims 1-53, wherein the subject is administered the AA conjugated to an agent at a fixed dose of about 280 mg to about 700 mg.

70. The method of any one of claims 1-53, wherein the subject is administered the AA conjugated to an agent at a fixed dose of about 320 mg to about 800 mg.

71. The method of any one of claims 1-53, wherein the subject is administered the AA conjugated to an agent at a fixed dose of about 360 mg to about 900 mg.

72. The method of any one of claims 1-53, wherein the subject is administered the AA conjugated to an agent at a fixed dose of about 400 mg to about 1000 mg.

73. The method of any one of claims 1-53, wherein the subject is administered the AA conjugated to an agent at a fixed dose of greater than 240 mg to about 280 mg.

74. The method of any one of claims 1-53, wherein the subject is administered the AA conjugated to an agent at a fixed dose of about 280 mg to about 320 mg.

75. The method of any one of claims 1-53, wherein the subject is administered the AA conjugated to an agent at a fixed dose of about 320 mg to about 360 mg.

76. The method of any one of claims 1-53, wherein the subject is administered the AA conjugated to an agent at a fixed dose of about 360 mg to about 400 mg.

77. The method of any one of claims 1-53, wherein the subject is administered the AA conjugated to an agent at a fixed dose of greater than 600 mg to about 700 mg.

78. The method of any one of claims 1-53, wherein the subject is administered the AA conjugated to an agent at a fixed dose of about 700 mg to about 800 mg.

79. The method of any one of claims 1-53, wherein the subject is administered the AA conjugated to an agent at a fixed dose of about 800 mg to about 900 mg.

80. The method of any one of claims 1-53, wherein the subject is administered the AA conjugated to an agent at a fixed dose of about 900 mg to about 1000 mg.

81. The method of any one of claims 1-53, wherein the subject is administered the AA conjugated to an agent at a fixed dose of greater than 240 mg to about 320 mg.

82. The method of any one of claims 1-53, wherein the subject is administered the AA conjugated to an agent at a fixed dose of about 280 mg to about 360 mg.

83. The method of any one of claims 1-53, wherein the subject is administered the AA conjugated to an agent at a fixed dose of about 320 mg to about 400 mg.

84. The method of any one of claims 1-53, wherein the subject is administered the AA conjugated to an agent at a fixed dose of greater than 600 mg to about 800 mg.

85. The method of any one of claims 1-53, wherein the subject is administered the AA conjugated to an agent at a fixed dose of about 700 mg to about 900 mg.

86. The method of any one of claims 1-53, wherein the subject is administered the AA conjugated to an agent at a fixed dose of about 800 mg to about 1000 mg.

87. The method of any one of claims 1-86, wherein the subject is administered the AA conjugated to an agent intravenously.

88. The method of any one of claims 1-87, wherein the subject is administered the AA conjugated to an agent intravenously every 21 days.

89. The method of any one of claims 1-87, wherein the subject is administered the AA conjugated to an agent intravenously every 14 days.

90. The method of any one of claims 54-64 and 87-89, wherein the subject is administered the AA conjugated to an agent with a dosage based on the subject's actual body weight.

91. The method of any one of claims 54-64 and 87-89, wherein the subject is administered the AA conjugated to an agent with a dosage based on the subject's adjusted ideal body weight.

92. The method of any one of claims 1-91, wherein the subject has not had a history of acute or chronic corneal disease.

93. The method of any one of claims 1-92, wherein the method comprises administering to the subject a prophylactic treatment to reduce or prevent ocular adverse events.

94. The method of claim 93, wherein the prophylactic treatment is administered daily.

95. The method of claim 93 or claim 94, wherein the prophylactic treatment is one or more treatments selected from the group consisting of: lubricating artificial tears, brimonidine tartrate ophthalmic solution, application of a cool compress for the eyes, and topical steroid drops.
